(19) United States
(12) Patent Application Publication
(10) Pub. No.: US 2022/0220189 A1
(43) Pub. Date: Jul. 14, 2022

(54) COMPOSITIONS AND METHODS FOR TREATMENT OF HEMOCHROMATOSIS

(71) Applicant: ULTRAGENYX PHARMACEUTICAL INC., Novato, CA (US)

(72) Inventors: Sean Christopher DAUGHERTY, Petaluma, CA (US); Timothy Preston WONG, Concord, CA (US); Rosaline Do CARSON, San Rafael, CA (US); Jason Robert CATALDO, Old Saybrook, CT (US)

(21) Appl. No.: 17/613,368

(22) PCT Filed: May 22, 2020
(86) PCT No.: PCT/US20/34377

§ 371 Date: Nov. 22, 2021

Related U.S. Application Data
(60) Provisional application No. 62/852,549, filed on May 24, 2019.
(60) Provisional application No. 62/991,907, filed on Mar. 19, 2020.

(51) Int. Cl. C07K 14/74; A01K 67/027; A61K 31/7088

(52) U.S. Cl. CPC C07K 14/70539; A01K 67/0276; A01K 2267/0306; A01K 2217/075; A01K 2227/105; A61K 31/7088

(57) ABSTRACT

This application provides polynucleotides comprising a coding sequence for a functionally active hereditary hemochromatosis protein (HFE) or a functionally active fragment thereof. The invention further provides compositions comprising said polynucleotides and their use in methods of preventing or treating hemochromatosis in a subject.

### CROSS-REFERENCE TO RELATED APPLICATIONS

This application claims the benefit of U.S. provisional application No. 62/852,549, filed on May 24, 2019, and U.S. provisional application 62/991,907, filed on Mar. 19, 2020, the contents of each of which are hereby incorporated by reference in their entirety.

### TECHNICAL FIELD OF THE INVENTION

This invention relates to nucleic acid molecules encoding a hereditary hemochromatosis protein (HFE) and compositions comprising the same for use in the treatment of hemochromatosis.

### BACKGROUND OF THE INVENTION

Hereditary hemochromatosis (HH), also known as type 1 hemochromatosis or genetic hemochromatosis, is an autosomal recessive disorder that results from a mutated HFE protein (also known as the hereditary hemochromatosis protein). A mutation in the HFE protein causes increased intestinal absorption of iron despite a normal dietary intake, leading to an abundance of iron deposition in the body, particularly in the liver, pancreas, heart, thyroid, pituitary gland, and joints. Excess iron deposition, if left untreated, causes tissue damage and fibrosis with the potential for hepatic cirrhosis, diabetes, arthropathy, congestive heart failure, hypogonadism, and skin hyperpigmentation.

Over 30 different disease-causing HFE mutations have been identified in HH, including Cys282Tyr (C282Y), His63Asp (H63D), and Ser65Cys (S65C). The most prevalent mutation is the 845G polymorphism, which causes the Cys282Tyr amino acid substitution (i.e., C282Y) in the HFE protein. The C282Y mutation disrupts the formation of a disulfide bond in the HFE protein and impairs its ability to bind 02-microglobulin. As a result, the HFE protein is unable to reach the cell surface and aggregates intracellularly, which causes impaired signaling leading to reduced hepcidin mRNA expression, decreased plasma hepcidin levels, and excessive systemic iron accumulation. The genetic disease can be recognized during its early stages when iron overload and organ damage are minimal. At this stage, the disease is best referred to as early or precirrhotic hemochromatosis.

The current standard of care for HH is phlebotomy. By drawing off red blood cells, the major mobilizer of iron in the body, iron toxicity can be minimized. Patients may require more than 100 phlebotomies of 500 mL each to reduce iron levels to normal. Phlebotomy is usually performed once or twice a week for up to three years during an induction phase. Once excess bodily iron has been removed and ferritin levels reach a steady value of less than 50 μg/L, lifelong, but less frequent, phlebotomy (typically 4-8 times a year) is required during the maintenance phase to keep serum ferritin levels below 50 μg/L.

Although phlebotomy may be an effective therapy for some patients, there is still a subset of patients that are ineligible for phlebotomies due to poor venous access, low blood pressure, congestive heart failure, or complications associated with HH. Moreover, some HH patients may be poorly compliant with phlebotomies (e.g., due to needlephobia) or may suffer from post-treatment anemia, bruising, and/or light-headedness.

Given the complications associated with phlebotomy, there is a need for an alternative therapeutic approach for HH, particularly for patients unwilling or unable to initiate or maintain a phlebotomy regimen.

In addition to HH, there is another form of hemochromatosis known as secondary hemochromatosis which can occur in patients who have hemoglobinopathies (e.g., sickle cell disease, thalassemia, and sideroblastic anemias), congenital hemolytic anemias, and myelodysplasia. In patients with secondary hemochromatosis (also known as secondary iron overload), iron overload results from increased iron absorption, exogenous iron given to treat anemia, and repeated blood transfusions. Increased iron absorption in some of these patients may be attributable to the deficiency or suppression of hepcidin, an inhibitor of iron absorption. See Papanikolaou et al., 2005, Blood 105(10): 4103-5. Indeed, Kautz et al. showed that erythroferrone (EFRE), an erythroid regulator of hepcidin synthesis and iron homeostasis, is expressed at abnormally high levels in a mouse model of β-thalassemia, and that ERFE can mediate suppression of hepcidin mRNA expression and contribute to iron overload. See Kautz et al., 2015, Blood 126(17): 2031-7. Secondary hemochromatosis is usually treated with iron chelators such as deferoxamine or deferasirox, but unfortunately, these therapies, can be complex to administer, require an unusual time commitment from patients, and/or are associated with adverse effects such as hypotension, GI disturbances, vision and hearing loss, and abnormal liver and kidney function. Thus, there is also a need for an alternative therapeutic approach for patients with secondary hemochromatosis.

The present invention addresses the need for an alternative therapeutic approach for hemochromatosis by providing nucleic acid molecules that have the ability to be translated to provide functional HFE protein, which can ameliorate, prevent or treat a disease or condition associated with the deficiency of functional HFE protein, such as HH, or other diseases or conditions associated with the reduction or suppression of hepcidin, such as secondary hemochromatosis.

### SUMMARY OF THE INVENTION

This invention provides compositions comprising novel nucleic acid molecules that can be used to provide functionally active proteins, or fragments thereof. The invention further provides methods of using these compositions comprising novel nucleic acid molecules for the prevention or treatment of various disorders, including hereditary hemochromatosis (HH) and secondary hemochromatosis. More specifically, embodiments of this invention provide compositions comprising translatable nucleic acid molecules to provide a functionally active hereditary hemochromatosis protein (HFE protein), or a functionally active fragment thereof, and methods of their use for the treatment of hemochromatosis. In some embodiments, the nucleic acid molecules of the invention can be expressible to provide an HFE protein product that is functionally active for ameliorating, preventing or treating a disease or condition associated with an HFE protein deficiency, such as HH, or other diseases or conditions associated with the reduction or suppression of hepcidin, such as secondary hemochromatosis.

In a first aspect, the application relates to a polynucleotide comprising an mRNA coding sequence for the hereditary hemochromatosis protein (HFE protein) or a fragment thereof. In one embodiment, the polynucleotide comprises a mixture of natural and modified nucleotides. Thus, in some embodiments, the application relates to a polynucleotide for expressing a human hereditary hemochromatosis protein (HFE), or a fragment thereof, wherein the polynucleotide comprises natural and modified nucleotides and is expressible to provide the human HFE or a fragment thereof having HFE activity.

In one embodiment, the mRNA coding sequence for the HFE protein is a wild-type coding sequence. In an alternative embodiment, the mRNA coding sequence for the HFE protein is a codon-optimized sequence. In one exemplary embodiment, the mRNA coding sequence for the HFE protein is codon-optimized for expression in humans.

In some embodiments, the HFE protein is encoded by the wild-type coding sequence shown in SEQ ID NO: 1. In another embodiment, a coding sequence expressing a natural isoform of the HFE protein may be used, such as an HFE protein shown in UniProtKB/Swiss-Prot Accession Nos. Q6BOJ5 (SEQ ID NO: 2) or F8W7W8 (SEQ ID NO: 3). In alternative embodiments, the HFE protein is encoded by a codon-optimized coding sequence that is less than 95% identical to the wild-type coding sequence shown in SEQ ID NO: 1. In some exemplary embodiments, the HFE protein is encoded by a codon-optimized coding sequence that comprises or consists of a nucleic acid selected from SEQ ID NOs: 4-31. In some embodiments, the polynucleotide comprising an mRNA coding sequence for the HFE protein further comprises a stop codon (UGA, UAA, or UAG) immediately downstream of the codon-optimized coding sequence. In some embodiments, the expressed HFE protein comprises or consists of an amino acid sequence of SEQ ID NO: 32 (GenBank Accession No. NP_000401.1, UniProtKB Accession No. Q30201, 348 amino acids). In some embodiments, the expressed polypeptide is a fragment of SEQ ID NO: 32 that retains functional HFE activity.

In some embodiments, the polynucleotide comprising an mRNA coding sequence for the HFE protein or a fragment thereof further comprises a 5′-cap. In one embodiment, the 5′-cap comprises N7-Methyl-Gppp(2′-O-Methyl-A). As will be appreciated by the skilled artisan, the 5′-cap can provide an A residue at the 5′ end of an RNA oligomer.

In some embodiments, the polynucleotide comprising an mRNA coding sequence for the HFE protein or a fragment thereof further comprises a 5′ untranslated region (5′ UTR) sequence. In one embodiment, the 5′ UTR sequence is selected from SEQ ID NOs: 33-34. In an exemplary embodiment, the 5′ UTR sequence comprises or consists of SEQ ID NO: 33.

In some embodiments, the polynucleotide comprising an mRNA coding sequence for the HFE protein or a fragment thereof further comprises a 3′ untranslated region (3′ UTR) sequence. In one embodiment, the 3′ UTR sequence is selected from SEQ ID NOs: 35-36. In an exemplary embodiment, the 3′ UTR sequence comprises or consists of SEQ ID NO: 35.

In some embodiments, the polynucleotide comprising an mRNA coding sequence for the HFE protein or a fragment thereof further comprises a comprises a 3′ polyA tail sequence. In some embodiments, the length of the polyA tail sequence can be at least about 5, 10, 15, 20, 25, 30, 35, 40, 45, 50, 100, 200, or 300 nucleotides. In some embodiments, a 3′ polyA tail sequence contains about 5 to 300 adenosine nucleotides (e.g., about 30 to 250 adenosine nucleotides, about 60 to 220 adenosine nucleotides, about 80 to 200 adenosine nucleotides, about 90 to about 150 adenosine nucleotides, or about 100 to about 120 adenosine nucleotides). In some embodiments, the 3′ polyA tail sequence is 60 to 220 adenosine nucleotides. In an exemplary embodiment, the 3′ polyA tail sequence is about 80 nucleotides in length. In another exemplary embodiment, the 3′ polyA tail sequence is about 100 nucleotides in length. In yet another exemplary embodiment, the 3′ polyA tail sequence is about 115 nucleotides in length.

In one embodiment, the polynucleotide comprising an mRNA coding sequence for the hereditary hemochromatosis protein (HFE protein) or a fragment thereof contains one or more modified nucleotides selected from 5-hydroxycytidine, 5-methylcytidine, 5-hydroxymethylcytidine, 5-carboxycytidine, 5-formylcytidine, 5-methoxycytidine, 5-propynylcytidine, 2-thiocytidine, 5-hydroxyuridine, 5-methyluridine, 5,6-dihydro-5-methyluridine, 2′-O-methyluridine, 2′-O-methyl-5-methyluridine, 2′-fluoro-2′-deoxyuridine, 2′-amino-2′-deoxyuridine, 2′-azido-2′-deoxyuridine, 4-thiouridine, 5-hydroxymethyluridine, 5-carboxyuridine, 5-carboxymethylesteruridine, 5-formyluridine, 5-methoxyuridine, 5-propynyluridine, 5-bromouridine, 5-iodouridine, 5-fluorouridine, pseudouridine, 2′-O-methyl-pseudouridine, N¹-hydroxypseudouridine, N¹-methylpseudouridine, 2′-O-methyl-N¹-methylpseudouridine, N¹-ethylpseudouridine, N¹-hydroxymethylpseudouridine, arauridine, N⁶-methyladenosine, 2-aminoadenosine, 3-methyladenosine, 7-deazaadenosine, 8-oxoadenosine, inosine, thienoguanosine, 7-deazaguanosine, 8-oxoguanosine, and 6-O-methylguanine.

In one embodiment, the polynucleotide comprises one or more pseudouridines. In some embodiments, the pseudouridine residue is selected from N¹-methylpseudouridine, N¹-ethylpseudouridine, N¹-propylpseudouridine, N¹-cyclopropylpseudouridine, N¹-phenylpseudouridine, N¹-aminomethylpseudouridine, N³-methylpseudouridine, N¹-hydroxypseudouridine, and N¹-hydroxymethylpseudouridine. In an exemplary embodiment, the polynucleotide is fully modified to comprise N¹-methylpseudouridine residues in place of uridine residues.

In an alternative embodiment, the polynucleotide comprises one or more modified nucleotides selected from 5-hydroxyuridine, 5-methyluridine, 5-hydroxymethyluridine, 5-carboxyuridine, 5-carboxymethylesteruridine, 5-formyluridine, 5-methoxyuridine, 5-propynyluridine, 5-bromouridine, 5-fluorouridine, 5-iodouridine, 2-thiouridine, and 6-methyluridine. In an exemplary embodiment, the polynucleotide is fully modified to comprise 5-methoxyuridine residues in place of uridine residues.

In some embodiments, the polynucleotide may comprise a mixture of modified nucleotides, e.g., a mixture of 5-methoxyuridine and N¹-methylpseudouridine residues in place of uridine residues.

In another aspect, the application provides novel codon-optimized mRNA sequences encoding HFE. In some embodiments, the codon-optimized nucleic acid sequence encoding HFE is at least 80%, 81%, 82%, 83%, 84%, 85%, 86%, 87%, 88%, 89%, 90%, 91%, 92%, 93%, 94%, 95%, 96%, 97%, 98%, 99%, 99.5%, 99.9%, or more identical to SEQ ID NOs: 4-31. In some embodiments, the application provides nucleic acid sequences encoding HFE which are less than 80%, 85%, 90%, 91%, 92%, 93%, 94%, or 95% identical to the wild-type coding sequence shown in SEQ ID NO: 1. In exemplary embodiments, the application provides a nucleic acid sequence encoding HFE that comprises or consists of a sequence selected from SEQ ID NOs: 4-31. Further provided are fragments of the nucleic acid sequences shown in SEQ ID NOs: 4-31 which encode a polypeptide having functional HFE activity. In some embodiments, the nucleic acid sequence may further comprise a stop codon (UGA, UAA, or UAG) at the 3′ end.

In yet another aspect, the application relates to a polynucleotide comprising or consisting of a nucleobase sequence that is less than 95% identical to the wild-type human HFE coding sequence over the full length human HFE coding sequence of SEQ ID NO: 1, and wherein the human HFE coding sequence is at least 95% identical to a sequence selected from SEQ ID NOs: 4-31. In an exemplary embodiment, the application relates to a polynucleotide comprising a nucleobase sequence of SEQ ID NO: 4.

In yet another aspect, the application relates to a polynucleotide comprising or consisting of a nucleobase sequence is at least 95% identical to a sequence selected from SEQ ID NOs: 4-31. In one embodiment, the application relates to a polynucleotide that comprises or consists of a nucleobase sequence is at least 95% identical to a sequence selected from SEQ ID NOs: 4-31. In another embodiment, the application relates to a polynucleotide that comprises or consists of a nucleobase sequence is at least 98% identical to a sequence selected from SEQ ID NOs: 4-31. In yet another embodiment, the application relates to a polynucleotide that comprises or consists of a nucleobase sequence is at least 99% identical to a sequence selected from SEQ ID NOs: 4-31. In yet another embodiment, the application relates to a polynucleotide that comprises or consists of a nucleobase sequence selected from SEQ ID NOs: 4-31.

In additional aspects, the application provides novel codon-optimized DNA sequences that can be transcribed to provide mRNA sequences encoding HFE. Accordingly, the application additionally relates to nucleic acid sequences which are at least 80%, 81%, 82%, 83%, 84%, 85%, 86%, 87%, 88%, 89%, 90%, 91%, 92%, 93%, 94%, 95%, 96%, 97%, 98%, 99%, 99.5%, 99.9%, or more identical to SEQ ID NOs: 37-64. In exemplary embodiments, the application provides a nucleic acid sequence that can be transcribed to provide an mRNA sequence encoding HFE selected from SEQ ID NOs: 37-64. Further provided are fragments of the nucleic acid sequences shown in SEQ ID NOs: 37-64 which can be transcribed to provide an mRNA sequence encoding a polypeptide having functional HFE activity. In some embodiments, the codon-optimized DNA sequence may further comprise a stop codon (TGA, TAA, or TAG) at the 3′ end.

In another aspect, the application relates to pharmaceutical compositions comprising (1) a polynucleotide comprising an mRNA coding sequence for the HFE protein or a fragment thereof, and (2) a pharmaceutically acceptable carrier. In some embodiments, the pharmaceutically acceptable carrier is selected from a transfection reagent, a nanoparticle (e.g., a lipid nanoparticle), or a liposome.

In an exemplary embodiment, the pharmaceutically acceptable carrier is a lipid nanoparticle. In an exemplary embodiment, the lipid nanoparticle comprises a cationic lipid, an aggregation reducing agent (such as polyethylene glycol (PEG) lipid or PEG-modified lipid), a non-cationic lipid (such as a neutral lipid), and a sterol. In a further exemplary embodiment, the lipid nanoparticle comprises at least one cationic lipid, a non-cationic lipid, a sterol, e.g., cholesterol, and a PEG-lipid, in a molar ratio of about 20-60% cationic lipid: 5-25% non-cationic lipid: 25-55% sterol: 0.5-15% PEG-lipid. In yet another embodiment, the cationic lipid is selected from ATX-002, ATX-081, ATX-095, and ATX-126 as described in the detailed description that follows.

In further aspects, the application relates to the use of a pharmaceutical composition comprising (1) a polynucleotide comprising an mRNA coding sequence for the HFE protein or a fragment thereof, and (2) a pharmaceutically acceptable carrier in medical therapy, e.g., in the treatment of the human or animal body.

In another aspect, the application relates to the use of a pharmaceutical composition comprising (1) a polynucleotide comprising an mRNA coding sequence for the HFE protein or a fragment thereof, and (2) a pharmaceutically acceptable carrier for preparing or manufacturing a medicament for ameliorating, preventing, delaying onset, or treating a disease or disorder associated with reduced activity of hereditary hemochromatosis protein (HFE) in a subject need thereof. In one embodiment, the disease or disorder is hereditary hemochromatosis.

In yet another aspect, the application relates to a method for ameliorating, preventing, delaying onset, or treating a disease or disorder associated with reduced activity of hereditary hemochromatosis protein (HFE) in a subject need thereof, the method comprising administering to the subject a pharmaceutical composition comprising (1) a polynucleotide comprising an mRNA coding sequence for the HFE protein or a fragment thereof, and (2) a pharmaceutically acceptable carrier. In one embodiment, the disease or disorder is hereditary hemochromatosis.

In yet another aspect, the application relates to methods of treating hemochromatosis in a human subject comprising administering to the human subject a therapeutically effective amount of at a pharmaceutical composition of the invention, e.g., a pharmaceutical composition comprising (1) a polynucleotide comprising an mRNA coding sequence for the HFE protein or a fragment thereof, and (2) a pharmaceutically acceptable carrier. In one embodiment, the hemochromatosis is hereditary hemochromatosis (HH). In one embodiment, the hemochromatosis is secondary hemochromatosis. In one embodiment, the application provides a method of treating hemochromatosis in a human subject comprising administering to the human subject a pharmaceutical composition comprising (1) a polynucleotide comprising an mRNA coding sequence for the HFE protein or a fragment thereof, and (2) a pharmaceutically acceptable carrier. In an exemplary embodiment, the pharmaceutically acceptable carrier is a lipid nanoparticle. In a further exemplary embodiment, the nanoparticle comprises a cationic lipid, an aggregation reducing agent (such as polyethylene glycol (PEG) lipid or PEG-modified lipid), a non-cationic lipid (such as a neutral lipid), and a sterol. In another further exemplary embodiment, the nanoparticle comprises at least one cationic lipid, a non-cationic lipid, a sterol, e.g., cholesterol, and a PEG-lipid, in a molar ratio of about 20-60% cationic lipid: 5-25% non-cationic lipid: 25-55% sterol: 0.5-15% PEG-lipid. In some embodiments, the cationic lipid is selected from ATX-002, ATX-081, ATX-095, and ATX-126. In some embodiments, the pharmaceutical composition comprises a polynucleotide comprising a codon-optimized nucleic acid sequence encoding HFE which is at least 80%, 81%, 82%, 83%, 84%, 85%, 86%, 87%, 88%, 89%, 90%, 91%, 92%, 93%, 94%, 95%, 96%, 97%, 98%, 99%, 99.5%, 99.9%, or more identical to SEQ ID NOs: 4-31. In an exemplary embodiment, the pharmaceutical composition comprises a polynucleotide comprising a codon-optimized nucleic acid sequence encoding HFE which is at least 95% identical to SEQ ID NOs: 4. In another exemplary embodiment, the pharmaceutical composition comprises a polynucleotide comprising a codon-optimized nucleic acid sequence encoding HFE which is at least 98% identical to SEQ ID NOs: 4.

In yet another aspect, the application relates to methods of treating hereditary hemochromatosis in a human subject comprising administering to a human subject diagnosed with at least one mutation in HFE a therapeutically effective amount of a pharmaceutical composition described herein.

In some embodiments, a pharmaceutical composition of the invention is administered via intravenous, subcutaneous, pulmonary, intramuscular, intraperitoneal, dermal, oral, nasal, or inhalational administration.

In some embodiments, a pharmaceutical composition of the invention is administered once daily, weekly, every two weeks, monthly, every two months, quarterly, or yearly.

In some embodiments, a pharmaceutical composition of the invention administered at a dose of about 0.01 to about 10 mg/kg. In some embodiments, a pharmaceutical composition of the invention is administered at a dose of about 0.1, 0.3, 0.5, 1, 3, 5, or about 10 mg/kg.

In yet another aspect, the application relates to a kit for expressing a human HFE in vivo. In one embodiment, the kit comprises a 0.1 to 500 mg dose of one or more polynucleotides of the invention and a device for administering the dose. In one embodiment, the device is an injection needle, an intravenous needle, or an inhalation device.

These and other aspects and features of the invention are described in the following sections of the application.

### BRIEF DESCRIPTION OF THE DRAWINGS

FIG. 1 shows a western blot from lysates isolated from human primary hepatocytes transfected with mRNA coding for the human HFE protein at varying concentrations 24-hours post-transfection. This demonstrates the ability for mRNA constructs to produce significant amounts of HFE protein in vitro.

FIG. 2 shows western blot data from lysates isolated at varying time points (days 1-6 post-transfection) from human primary hepatocytes transfected with 500 ng of mRNA coding for the human HFE protein. This demonstrates the substantial duration of HFE expression following a single transfection of HFE-encoding mRNA.

FIG. 3 shows western blot data from liver homogenates of Hfe knockout mice taken at 48 hours post-intravenous administration of lipid nanoparticle encapsulated HFE-encoding mRNA. mRNA was dosed at 0.3 mg/kg, 1 mg/kg, and 3 mg/kg. This demonstrates that liver HFE protein expression can be detected in a dose-dependent manner following a single dose of HFE-encoding mRNA.

FIG. 4 shows liver hepcidin expression in Hfe knockout mice at 48 hours post-intravenous administration of lipid nanoparticle encapsulated HFE-encoding mRNA. mRNA was dosed at 0.3 mg/kg, 1 mg/kg, and 3 mg/kg. This demonstrates that liver hepcidin expression can be re-established following a single dose of HFE-encoding mRNA.

FIG. 5 shows serum iron levels in Hfe knockout mice at 48 hours post-intravenous administration of lipid nanoparticle encapsulated HFE-encoding mRNA. mRNA was dosed at 0.3 mg/kg (female=F), 1 mg/kg (female=F), and 3 mg/kg (male=M). This demonstrates that peripheral iron levels are reduced in response to a single dose of HFE-encoding mRNA.

FIG. 6 shows blood transferrin (Tf) saturation levels in Hfe knockout mice at 48 hours post-intravenous administration of lipid nanoparticle encapsulated HFE-encoding mRNA. mRNA was dosed at 0.3 mg/kg (female=F), 1 mg/kg (female=F), and 3 mg/kg (male=M). This demonstrates that Tf saturation levels are reduced in response to a single dose of HFE-encoding mRNA.

FIG. 7 shows liver iron levels in Hfe knockout mice at 7 days post-intravenous administration of lipid nanoparticle encapsulated HFE-encoding mRNA. mRNA was dosed at 1 mg/kg. Liver iron levels were reduced in female (F) and male (M) mice in treated groups relative to animals treated with vehicle (veh) control.

### DETAILED DESCRIPTION OF THE INVENTION

This invention provides a range of novel agents and compositions to be used for therapeutic applications. In some embodiments, the nucleic acid molecules and compositions of this invention can be used for ameliorating, preventing or treating hereditary hemochromatosis and/or any additional diseases associated reduced presence or function of the hereditary hemochromatosis protein (HFE) in a subject. In other embodiments, the nucleic acid molecules and compositions of this invention can be used for ameliorating, preventing or treating secondary hemochromatosis.

In some embodiments, this invention encompasses synthetic, purified, translatable polynucleotide molecules for expressing a human hereditary hemochromatosis protein. The molecules may contain natural and modified nucleotides, and encode the human hereditary hemochromatosis protein (HFE), or a fragment thereof having HFE activity.

As used herein, the term “translatable” may be used interchangeably with the term “expressible” and refers to the ability of polynucleotide, or a portion thereof, to be converted to a polypeptide by a host cell. As is understood in the art, translation is the process in which ribosomes in a cell's cytoplasm create polypeptides. In translation, messenger RNA (mRNA) is decoded by tRNAs in a ribosome complex to produce a specific amino acid chain, or polypeptide. Furthermore, the term “translatable” when used in this specification in reference to an oligomer, means that at least a portion of the oligomer, e.g., the coding region of an oligomer sequence (also known as the coding sequence or CDS), is capable of being converted to a protein or a fragment thereof.

As used herein, the term “monomer” refers to a single unit, e.g., a single nucleic acid, which may be joined with another molecule of the same or different type to form an oligomer.

Meanwhile, the term “oligomer” may be used interchangeably with “polynucleotide” and refers to a molecule comprising at least two monomers and includes oligonucleotides such as DNAs and RNAs. In the case of oligomers containing RNA monomers, the oligomers of the present invention may contain sequences in addition to the coding sequence (CDS). These additional sequences may be untranslated sequences, i.e., sequences which are not converted to protein by a host cell. These untranslated sequences can include a 5′-cap or a portion thereof, a 5′ untranslated region (5′ UTR), a 3′ untranslated region (3′ UTR), and a tail region, e.g., a polyA tail region. In the context of the present invention, a “translatable oligomer”, a “translatable molecule”, “translatable polynucleotide”, or “translatable compound” refers to a sequence that comprises a region, e.g., the coding region of an RNA (e.g., the coding sequence of human HFE or a codon-optimized version thereof), that is capable of being converted to a protein or a fragment thereof, e.g., the human HFE protein or a fragment thereof.

As used herein, the term “codon-optimized” means a natural (or purposefully designed variant of a natural) coding sequence which has been redesigned by choosing different codons without altering the encoded protein amino acid sequence increasing the protein expression levels (Gustafsson et al., 2004, Trends Biotechnol 22: 346-53). Variables such as high codon adaptation index (CAI), LowU method, mRNA secondary structures, cis-regulatory sequences, GC content and many other similar variables have been shown to somewhat correlate with protein expression levels (Villalobos et al., 2006, BMC Bioinformatics 7:285). The high CAI (codon adaptation index) method picks a most frequently used synonymous codon for an entire protein coding sequence. The most frequently used codon for each amino acid is deduced from 74218 protein-coding genes from a human genome. The LowU method targets only U-containing codons that can be replaced with a synonymous codon with fewer U moieties. If there are a few choices for the replacement, the more frequently used codon will be selected. The remaining codons in the sequence are not changed by the LowU method. This method may be used in conjunction with the disclosed mRNAs to design coding sequences that are to be synthesized with one or more modified nucleotides such as N¹-methylpseudouridine or 5-methoxyuridine.

As will be appreciated by the skilled artisan equipped with the present disclosure, the polynucleotides of the present invention and compositions comprising the same may be used to ameliorate, prevent, or treat any disease or disorder associated with reduced activity (e.g., resulting from reduced concentration, presence, and/or function) of the hereditary hemochromatosis protein (HFE protein) in a subject. In some embodiments, the polynucleotides of this invention can be used in methods for ameliorating, preventing or treating hereditary hemochromatosis (HH). The disease or disorder to be treated herein (e.g., HH) may be associated with excess iron deposition, tissue damage, fibrosis, hepatic cirrhosis, diabetes, arthropathy, congestive heart failure, hypogonadism, and skin hyper pigmentation. In some embodiments, the polynucleotides of the present invention and compositions comprising the same may be used to ameliorate, prevent, or treat any or all of these aforementioned symptoms.

As is understood by the skilled artisan, hereditary hemochromatosis (HH) may be referred to by any number of alternative names in the art, including, but not limited to, HFE deficiency, HFE hereditary hemochromatosis, HFE-related hereditary hemochromatosis, hemochromatosis type I, classic hemochromatosis, primary hemochromatosis, bronze diabetes, or hemosiderosis. Accordingly, HH may be used interchangeably with any of these alternative names in the specification, the examples, the drawings, and the claims.

As will be appreciated by the skilled artisan equipped with the present disclosure, the polynucleotides of the present invention and compositions comprising the same may be also be useful for ameliorating, preventing, or treating any disease or disorder associated with the reduction or suppression of hepcidin in a subject. In some embodiments, the polynucleotides of this invention can be used in methods for ameliorating, preventing or treating secondary hemochromatosis. In some embodiments, the secondary hemochromatosis occurs in a patient with a hereditary or acquired disorder of erythropoiesis. In some embodiments, the disease is a hereditary disease, such as thalassemia (e.g., β-thalassemia), sickle-cell anemia, pyruvate kinase deficiency, congenital dyserythropoietic anemia (CDA), Diamond-Blackfan anemia, hereditary spherocytosis, or X-linked sideroblastic anemia (ALAS2 deficiency). In some embodiments, the disease is an acquired disease, such as acquired idiopathic sideroblastic anemia (AISA), certain myelodysplastic syndromes (MDS), myelofibrosis, and intractable aplastic anemia. In some embodiments, the secondary hemochromatosis may be associated with excess iron deposition, tissue damage, fibrosis, hepatic cirrhosis, diabetes, arthropathy, congestive heart failure, hypogonadism, and skin hyperpigmentation. In some embodiments, the polynucleotides of the present invention and compositions comprising the same may be used to ameliorate, prevent, or treat any or all of these aforementioned symptoms.

As is understood by the skilled artisan, secondary hemochromatosis (SH) may be used broadly to refer to, or encompass, all cases of iron overload that are not due to a primary, hereditary disorder of iron metabolism. See Gattermann, 2009, Dtsch Arztebl Int. 106(30): 499-504. Secondary hemochromatosis is almost always due a hereditary or acquired disorder of erythropoiesis and/or the treatment of such a disorder with blood transfusion. Secondary hemochromatosis (SH) may be referred to by any number of alternative names in the art, including, but not limited to, secondary iron overload and non-HFE hemochromatosis. Accordingly, secondary hemochromatosis (SH) may be used interchangeably with any of these alternative names in the specification, the examples, the drawings, and the claims.

A polynucleotide of this invention encoding a functional HFE protein or a functional fragment thereof can be delivered to the liver, in particular to hepatocytes, of a patient in need (e.g., a patient with HH or SH), and can elevate functionally active HFE levels of the patient. The polynucleotide and compositions comprising the same can be used for preventing, treating, ameliorating or reversing any symptoms of HH or SH in the patient. In an exemplary embodiment, the patient is a human.

In further aspects, a polynucleotide of this invention and a composition comprising the same can also be used for reducing the dependence of a HH patient on phlebotomies to control the disease. For instance, a polynucleotide of this invention and a composition comprising the same can be used to reduce the total number of phlebotomies (e.g., by reducing the weekly frequency or monthly/yearly duration) needed by a HH patient to maintain serum ferritin levels below 50 μg/L.

Embodiments of this invention further encompass processes for making a polynucleotide capable of expressing a human hereditary hemochromatosis protein (HFE). The processes include transcribing in vitro a HFE DNA template in the presence of natural and modified nucleoside triphosphates to form a product mixture, and purifying the product mixture to isolate the polynucleotide. In some embodiments, a polynucleotide of the invention may be made by methods known in the art. In some embodiments, the polynucleotides of this invention can display a sequence of nucleobases designed to express a polypeptide or protein, in vitro, ex vivo, or in vivo.

In some embodiments, a polynucleotide of this invention may comprise a 5′-cap, a 5′ untranslated region of monomers, a coding region of monomers, a 3′ untranslated region of monomers, and a tail region of monomers.

In some embodiments, a polynucleotide of the invention can be from about 200 to about 4,000 monomers in length. In certain embodiments, a polynucleotide of the invention can be from 800 to 2,000 monomers in length, from 1,000 to 1,600 monomers in length, or from 1,100 to 1,500 monomers in length. In an exemplary embodiment, the polynucleotide of the invention is from 1,200 to 1,400 monomers in length. In a further exemplary embodiment, the polynucleotide of the invention is about 1,300 monomers in length.

In some embodiments, the polynucleotide comprising an mRNA coding sequence for the HFE protein or a fragment thereof comprises a mixture of natural and modified nucleotides and is expressible to provide the human HFE or a fragment thereof having HFE activity. In some embodiments, the modified nucleotide is 5-methoxyuridine. In an exemplary embodiment, the polynucleotide is fully modified to comprise 5-methoxyuridine residues in place of uridine residues. In some embodiments, the modified nucleotide is N¹-methylpseudouridine. In an exemplary embodiment, the polynucleotide is fully modified to comprise N¹-methylpseudouridine residues in place of uridine residues. In some embodiments, the polynucleotide is modified to comprise a mixture of 5-methoxyuridine and N¹-methylpseudouridine residues in place of uridine residues.

In some embodiments, the polynucleotides of this invention may be translatable molecules containing RNA monomers and/or alternative monomers such as unlocked nucleic acid (UNA) and locked nucleic acid (LNA) monomers.

In some embodiments, a translatable polynucleotide can contain from 1 to about 80 unlocked nucleic acid (UNA) monomers. In certain embodiments, a translatable polynucleotide can contain from 1 to 50 UNA monomers, or 1 to 20 UNA monomers, or 1 to 10 UNA monomers.

In some embodiments, a translatable polynucleotide can contain from 1 to about 80 locked nucleic acid (LNA) monomers. In certain embodiments, a translatable polynucleotide can contain from 1 to 50 LNA monomers, or 1 to 20 LNA monomers, or 1 to 10 LNA monomers.

In some embodiments, one or more polynucleotides of the invention can be delivered to a cell, in vitro, ex vivo, or in vivo. Viral and non-viral transfer methods as are known in the art can be used to introduce polynucleotides of the inventions into mammalian cells. In exemplary embodiment, polynucleotides of the invention may be delivered with a pharmaceutically acceptable vehicle, for example, with nanoparticles or liposomes. In a further exemplary embodiment, polynucleotides of the invention are delivered via nanoparticles, e.g., lipid nanoparticles (LNPs).

In additional embodiments, this invention provides methods for treating a disease or condition in a subject by administering to the subject a composition containing a polynucleotide of the invention.

In some aspects, a composition comprising a polynucleotide of the invention may be used for ameliorating, preventing or treating a disease or disorder, e.g., a disease or disorder associated with reduced activity (e.g., resulting from reduced concentration, presence, and/or function) of hepcidin in a subject. In this aspect, a composition comprising a polynucleotide of this invention can be administered to regulate, modulate, or increase the concentration or effectiveness of the hepcidin in a subject. Diseases or disorders associated with reduced activity of hepcidin include HH and SH.

In some aspects, a composition comprising a polynucleotide of the invention may be used for ameliorating, preventing or treating a disease or disorder, e.g., a disease or disorder associated with reduced activity (e.g., resulting from reduced concentration, presence, and/or function) of the hereditary hemochromatosis protein (HFE) in a subject. In one embodiment, a composition comprising a polynucleotide of this invention can be administered to regulate, modulate, or increase the concentration or effectiveness of the HFE protein in a subject. In some embodiments, the HFE protein to be expressed can be an unmodified, natural protein for which the patient is deficient (e.g., a patient with a mutated version of HFE which partially or totally abolishes functional HFE activity). In some aspects, the HFE protein expressed by a polynucleotide of the invention can be identical to an unmodified, natural, functionally active HFE protein which can be used to treat HH in a patient harboring a mutated version of the HFE protein. In exemplary embodiments, a composition comprising a polynucleotide of this invention may be used for ameliorating, preventing or treating HH.

In some embodiments, a polynucleotide of the invention may be delivered to cells or subjects, and translated to increase HFE levels in the cell or subject.

As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

In an exemplary embodiment, a subject of the present invention is a subject with reduced activity (e.g., resulting from reduced concentration, presence, and/or function) of hepcidin. In another exemplary embodiment, a subject of the present invention is a subject with reduced activity (e.g., resulting from reduced concentration, presence, and/or function) of HFE. In a further exemplary embodiment, the subject is a human.

In some embodiments, administering a composition comprising a polynucleotide of the invention can result in an increase in the level of functionally active HFE protein in a treated subject. In some embodiments, administering a composition comprising a polynucleotide of the invention results in about a 5%, 10%, 20%, 30%, 40%, 50%, 60%, 70%, 80%, 90%, 100%, 200%, 500%, or more increase in the level of functionally active HFE protein relative to a baseline level in the subject prior to treatment. In an exemplary embodiment, administering a composition comprising a polynucleotide of the invention results in an increase in liver HFE levels relative to baseline liver HFE levels in the subject prior to treatment. In some embodiments, the increase in liver HFE levels can be at least about 5%, 10%, 20%, 30%, 40%, 50%, 100%, 200%, 500%, or more.

In some embodiments, the HFE protein which is expressed from a polynucleotide of the invention is detectable in the liver, serum, plasma, kidney, heart, muscle, brain, cerebrospinal fluid, or lymph nodes. In exemplary embodiments, the HFE protein is expressed in liver cells, e.g., hepatocytes of a treated subject.

In some embodiments, administering a composition comprising a polynucleotide of the invention results in the expression of a natural, non-mutated human HFE (i.e., normal or wild-type HFE as opposed to abnormal or mutated HFE) protein level at or above about 10 ng/mg, about 20 ng/mg, about 50 ng/mg, about 100 ng/mg, about 150 ng/mg, about 200 ng/mg, about 250 ng/mg, about 300 ng/mg, about 350 ng/mg, about 400 ng/mg, about 450 ng/mg, about 500 ng/mg, about 600 ng/mg, about 700 ng/mg, about 800 ng/mg, about 900 ng/mg, about 1000 ng/mg, about 1200 ng/mg or about 1500 ng/mg of the total protein in the liver of a treated subject.

As used herein, the term “about” or “approximately” as applied to one or more values of interest, refers to a value that is similar to a stated reference value. In certain embodiments, the term “approximately” or “about” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

In some embodiments, the expression of the natural, non-mutated, functionally active human HFE protein, or functionally active fragment thereof, is detectable after administration of a composition comprising a polynucleotide of the invention. In some embodiments, functionally active HFE protein is detectable 2, 4, 6, 12, 18, 24, 30, 36, 48, 60, and/or 72 hours after administration of a composition comprising a polynucleotide of the invention. In some embodiments, functionally active HFE protein is detectable 1 day, 2 days, 3 days, 4 days, 5 days, 6 days, and/or 7 days after administration of a composition comprising a polynucleotide of the invention. In some embodiments, functionally active HFE protein is detectable 1 week, 2 weeks, 3 weeks, and/or 4 weeks after administration of a composition comprising a polynucleotide of the invention. In some embodiments, functionally active HFE protein is detectable in the liver, e.g., hepatocytes, after administration of a composition comprising a polynucleotide of the invention.

### Human HFE

The human HFE gene encodes a 348 amino acid protein with a molecular mass of approximately 40.1 kDa. The HFE protein is a hepatocyte iron sensor and upstream regulator of hepcidin. HFE is indispensable for signaling to hepcidin and appears to act as a constituent of a larger iron-sensing complex. In this way, HFE is a key regulator in the liver for maintaining iron homeostasis. As noted above, genetic deficiency of normal, functional HFE activity can cause hereditary hemochromatosis (HH) attributed to chronic hyperabsorption of dietary iron which can lead to severe organ damage if left untreated.

The consensus human HFE mRNA coding sequence has a sequence of 1,044 nucleobases (absent the stop codon) and is shown in SEQ ID NO: 1. When translated, the consensus human HFE mRNA coding sequence encodes the 348 amino acid, wild-type HFE protein of SEQ ID NO: 32.

In some embodiments, a polynucleotide of the invention comprises an mRNA sequence capable of being translated into a functionally active human HFE protein or a fragment thereof which exhibits functional HFE activity. The polynucleotides of the invention expressing a functionally active human HFE protein may be suitable for use in methods for ameliorating, preventing or treating disease associated with deficiency of normal HFE activity.

In some embodiments, a polynucleotide of the invention may comprise a 5′-cap, a 5′ UTR, a human HFE coding sequence (CDS), a 3′UTR, and/or a tail region. In an exemplary embodiment, the polynucleotide may include a 5′-cap ((e.g., N7-Methyl-Gppp(2′-O-Methyl-A)), a 5′ UTR comprising or consisting of SEQ ID NO: 33, an HFE CDS, a 3′ UTR comprising or consisting of SEQ ID NO: 35, and/or a tail region. In further exemplary embodiments, the HFE CDS may comprise a codon-optimized sequence of SEQ ID NOs: 4-31, described in further detail below. In any of these and other embodiments described herein, the polynucleotide may comprise one or more modified nucleotides, e.g., 5-methoxyuridine and/or N¹-methylpseudouridine, in place of one or more (or all) uridine residues.

In some embodiments, the translation efficiency of the molecule can be increased as compared to a native mRNA of HFE. For example, the translational expression of a molecule can be increased by 5%, 10%, 20%, 30%, 40%, 50%, 100%, 200%, or more relative to a native mRNA of HFE.

In some embodiments, a suitable mRNA sequence for the present invention comprises an mRNA sequence encoding the HFE protein. The sequence of the naturally occurring, functionally active human HFE protein is shown in SEQ ID NO: 32.

In some embodiments, a suitable mRNA sequence may be an mRNA sequence that encodes a homolog or variant of human HFE. As used herein, a homolog or a variant of human HFE protein may be a modified human HFE protein containing one or more amino acid substitutions, deletions, and/or insertions as compared to a wild-type or naturally-occurring human HFE protein while retaining substantial functional HFE protein activity. In some embodiments, an mRNA suitable for the present invention encodes a protein substantially identical to human HFE protein. In some embodiments, an mRNA suitable for the present invention encodes an HFE protein having an amino acid sequence at least 80%, 85%, 90%, 91%, 92%, 93%, 94%, 95%, 96%, 97%, 98%, 99% or more identical to SEQ ID NO: 32, wherein said HFE protein exhibits substantially equivalent or increased functional activity relative to the HFE protein having the amino acid sequence of SEQ ID NO: 32. In some embodiments, an mRNA suitable for the present invention encodes a functionally active fragment, a functionally active portion, or functionally active portions of a human HFE protein.

In some embodiments, an mRNA suitable for the present invention encodes a fragment or a portion of human HFE protein, wherein the fragment or portion of the protein still maintains HFE activity similar to that of the wild-type protein.

In some embodiments, an mRNA suitable for the present invention comprises a sequence that is at least 80%, 81%, 82%, 83%, 84%, 85%, 86%, 87%, 88%, 89%, 90%, 91%, 92%, 93%, 94%, 95%, 96%, 97%, 98%, 99%, 99.5%, 99.9%, or more identical to a sequence selected from SEQ ID NOs: 4-31. In an exemplary embodiment, an mRNA suitable for the present invention comprises a sequence that is at least 95% identical to a sequence selected from SEQ ID NOs: 4-31. In another exemplary embodiment, an mRNA suitable for the present invention comprises a sequence that is at least 98% identical to a sequence selected from SEQ ID NOs: 4-31. In a further exemplary embodiment, an mRNA suitable for the present invention comprises a sequence that is at least 98% identical to SEQ ID NO: 4.

In some embodiments, a polynucleotide of the present invention comprises a coding sequence that is at least 80%, 81%, 82%, 83%, 84%, 85%, 86%, 87%, 88%, 89%, 90%, 91%, 92%, 93%, 94%, 95%, 96%, 97%, 98%, 99%, 99.5%, 99.9%, or more identical to a sequence selected from SEQ ID NOs: 4-31. In some embodiments, a polynucleotide comprising a coding sequence that is at least 80%, 81%, 82%, 83%, 84%, 85%, 86%, 87%, 88%, 89%, 90%, 91%, 92%, 93%, 94%, 95%, 96%, 97%, 98%, 99%, 99.5%, 99.9%, or more identical to a sequence selected from SEQ ID NOs: 4-31 further comprises one or more sequences selected from a 5′-cap, a 5′ UTR, a 3′ UTR, and a tail region.

In some embodiments, a polynucleotide of the present invention comprises a coding sequence that is less than 80%, 81%, 82%, 83%, 84%, 85%, 86%, 87%, 88%, 89%, 90%, 91%, 92%, 93%, 94%, 95%, 96%, 97%, 98%, or 99% identical to the wild-type human HFE coding sequence over the full length human HFE coding sequence of SEQ ID NO: 1, and expresses a functionally active human HFE protein. In an exemplary embodiment, a polynucleotide of the present invention comprises a coding sequence that is less than 95% identical to the wild-type human HFE coding sequence over the full length human HFE coding sequence of SEQ ID NO: 1, and expresses a functional human HFE protein. In another exemplary embodiment, a polynucleotide of the present invention comprises a coding sequence that is less than 95% identical to the wild-type human HFE coding sequence over the full length human HFE coding sequence of SEQ ID NO: 1, and expresses a functional human HFE protein, wherein the coding sequence is at least 95% to a sequence selected from SEQ ID NOs: 4-31. Accordingly, in some embodiments, the present application provides a polynucleotide comprising of or consisting of a nucleobase sequence that is less than 95% identical to the wild-type human HFE coding sequence over the full length human HFE coding sequence of SEQ ID NO: 1, and wherein the human HFE coding sequence is at least 95%, 96%, 97%, 98%, 99% or more identical to a sequence selected from SEQ ID NOs: 4-31. In an exemplary embodiment, the present application provides a polynucleotide comprising of or consisting of a nucleobase sequence that is less than 95% identical to the wild-type human HFE coding sequence over the full length human HFE coding sequence of SEQ ID NO: 1, and wherein the human HFE coding sequence is at least 95% identical to a sequence selected from SEQ ID NOs: 4-31. In some embodiments, the polynucleotide may comprise a sequence selected from SEQ ID NOs: 4-31 and a stop codon (UGA, UAA, or UAG) immediately downstream of said sequence. In a specific embodiment, the present application provides a polynucleotide comprising a nucleobase sequence of SEQ ID NO: 4. In yet another specific embodiment, the present application provides a polynucleotide comprising a nucleotide base sequence of SEQ ID NO: 67.

In some embodiments, the application further provides novel codon-optimized DNA sequences that can be transcribed to provide mRNA sequences encoding HFE. Accordingly, the application additionally relates to nucleic acid sequences which are at least 95%, 96%, 97%, 98%, 99% or more identical to a sequence selected from SEQ ID NOs: 37-64. In exemplary embodiments, the application provides a nucleic acid sequence that can be transcribed to provide an mRNA sequence encoding HFE selected from SEQ ID NOs: 37-64. Further provided are fragments of the nucleic acid sequences shown in SEQ ID NOs: 37-64 which can be transcribed to provide an mRNA sequence encoding a polypeptide having functional HFE activity. In some embodiments, the polynucleotide may comprise a sequence selected from SEQ ID NOs: 37-64 and a stop codon (TGA, TAA, or TAG) immediately downstream of said sequence. In a specific embodiment, the present application provides a polynucleotide comprising a DNA sequence of SEQ ID NO: 37.

In some embodiments, a polynucleotide of the invention may comprise one or more unlocked nucleomonomers (i.e., UNA monomers). See, e.g., U.S. Pat. No. 9,944,929.

In some embodiments, a polynucleotide of the invention may comprise one or more locked nucleic acids (i.e., LNA monomers). See, e.g., U.S. Pat. Nos. 6,268,490, 6,670,461, 6,794,499, 6,998,484, 7,053,207, 7,084,125, 7,399,845, and 8,314,227.

In some embodiments, a polynucleotide of the invention encodes a fusion protein comprising a full length, fragment or portion of an HFE protein fused to another sequence (e.g., an N or C terminal fusion). In some embodiments, the N or C terminal sequence is a signal sequence or a cellular targeting sequence.

### Modified Nucleotides

In various embodiments described herein, a polynucleotide of the invention may comprise a combination of natural and modified nucleic acid monomers (i.e., nucleotides). Various examples of modified nucleotides are disclosed in WO/2018/222926, which is herein incorporated by reference in its entirety.

In some embodiments, an alkyl, cycloalkyl, or phenyl substituent may be unsubstituted, or further substituted with one or more alkyl, halo, haloalkyl, amino, or nitro substituents.

In some embodiments, a polynucleotide of the invention comprises one or more pseudouridines. Examples of pseudouridines include N¹-alkylpseudouridines, N¹-cycloalkylpseudouridines, N¹-hydroxypseudouridines, N¹-hydroxyalkylpseudouridines, N¹-phenylpseudouridines, N¹-phenylalkylpseudouridines, N¹-aminoalkylpseudouridines, N³-alkylpseudouridines, N⁶-alkylpseudouridines, N⁶-alkoxypseudouridines, N⁶-hydroxypseudouridines, N⁶-hydroxyalkylpseudouridines, N⁶-morpholinopseudouridines, N⁶-phenylpseudouridines, and N⁶-halopseudouridines. Examples of pseudouridines include N¹-alkyl-N⁶-alkylpseudouridines, N¹-alkyl-N⁶-alkoxypseudouridines, N¹-alkyl-N⁶-hydroxypseudouridines, N¹-alkyl-N⁶-hydroxyalkylpseudouridines, N¹-alkyl-N⁶-morpholinopseudouridines, N¹-alkyl-N⁶-phenylpseudouridines, and N¹-alkyl-N⁶-halopseudouridines. In these examples, the alkyl, cycloalkyl, and phenyl substituents may be unsubstituted, or further substituted with alkyl, halo, haloalkyl, amino, or nitro substituents. Examples of pseudouridines further include N¹-methylpseudouridine, N¹-ethylpseudouridine, N¹-propylpseudouridine, N¹-cyclopropylpseudouridine, N¹-phenylpseudouridine, N¹-aminomethylpseudouridine, N³-methylpseudouridine, N¹-hydroxypseudouridine, and N¹-hydroxymethylpseudouridine.

In some embodiments, the pseudouridine residue is selected from N¹-methylpseudouridine, N¹-ethylpseudouridine, N¹-propylpseudouridine, N¹-cyclopropylpseudouridine, N¹-phenylpseudouridine, N¹-aminomethylpseudouridine, N³-methylpseudouridine, N¹-hydroxypseudouridine, and N¹-hydroxymethylpseudouridine. In an exemplary embodiment, a polynucleotide of the invention is fully modified to comprise N¹-methylpseudouridine residues in place of uridine residues.

In some embodiments, a polynucleotide of the invention comprises one or more modified nucleotides selected from 5-hydroxyuridine, 5-methyluridine, 5-hydroxymethyluridine, 5-carboxyuridine, 5-carboxymethylesteruridine, 5-formyluridine, 5-methoxyuridine, 5-propynyluridine, 5-bromouridine, 5-fluorouridine, 5-iodouridine, 2-thiouridine, and 6-methyluridine. In an exemplary embodiment, a polynucleotide of the invention is fully modified to comprise 5-methoxyuridine residues in place of uridine residues.

In some embodiments, a polynucleotide of the invention may comprise one or more modified nucleotides selected from 2′-O-methyl ribonucleotides, 2′-O-methyl purine nucleotides, 2′-deoxy-2′-fluoro ribonucleotides, 2′-deoxy-2′-fluoro pyrimidine nucleotides, 2′-deoxy ribonucleotides, 2′-deoxy purine nucleotides, universal base nucleotides, 5-C-methyl-nucleotides, and inverted deoxyabasic monomer residues.

In some embodiments, a polynucleotide of the invention may comprise one or more modified nucleotides selected from 3′-end stabilized nucleotides, 3′-glyceryl nucleotides, 3′-inverted abasic nucleotides, and 3′-inverted thymidine.

In some embodiments, a polynucleotide of the invention may comprise one or more modified nucleotides selected from unlocked nucleic acid nucleotides (UNA), locked nucleic acid nucleotides (LNA), 2′-O,4′-C-methylene-(D-ribofuranosyl) nucleotides, 2′-methoxyethoxy (MOE) nucleotides, 2′-methyl-thio-ethyl, 2′-deoxy-2′-fluoro nucleotides, and 2′-O-methyl nucleotides. In one exemplary embodiment, the modified nucleotide is an unlocked nucleic acid nucleotide (UNA). A detailed summary of unlocked nucleic acids and methods for their incorporation into polynucleotides is found in WO/2018/222926, which is herein incorporated by reference in its entirety. In another exemplary embodiment, the modified nucleotide is a locked nucleic acid nucleotide (LNA).

In some embodiments, a polynucleotide of the invention may comprise one or more modified nucleotides selected from 2′,4′-constrained 2′-O-methoxyethyl (cMOE) and 2′-O-Ethyl (cEt) modified DNAs.

In some embodiments, a polynucleotide of the invention may comprise one or more modified nucleotides selected from 2′-amino nucleotides, 2′-O-amino nucleotides, 2′-C-allyl nucleotides, and 2′-O-allyl nucleotides.

Example of base modifications described above can be combined with additional modifications of nucleoside or nucleotide structure, including sugar modifications and linkage modifications.

### Molecular Cap Structure

In some embodiments, the polynucleotide comprising an mRNA coding sequence for the HFE protein or a fragment thereof further comprises a 5′-cap.

5′-caps and their analogues are known in the art. Some examples of 5′-cap structures are given in WO/2017/053297, WO/2015/051169, WO/2015/061491, and U.S. Pat. Nos. 8,093,367 and 8,304,529.

In one embodiment, the application provides 5′-capped RNAs, wherein the initiating capped oligonucleotide primers have the general form ^{m7}Gppp[N_{2′OME}]ₙ[N]ₘ wherein ^{m7}G is N7-methylated guanosine or any guanosine analog, N is any natural, modified or unnatural nucleoside, “n” can be any integer from 0 to 4 and “m” can be an integer from 1 to 9. Compositions and methods for synthesizing such 5′-capped RNAs are described in WO/2017/053797.

In an exemplary embodiment, the 5′-cap comprises N7-Methyl-Gppp(2′-O-Methyl-A).

In an exemplary embodiment, the 5′-cap has the following structure:

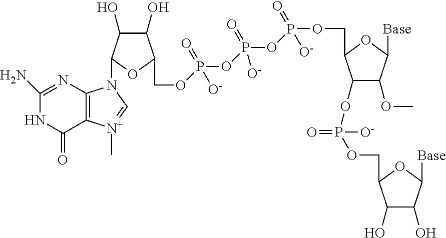

In some embodiments, the 5′-cap may be a m7GpppGm cap. In further embodiments, the 5′-cap may be selected from m7GpppA, m7GpppC; unmethylated cap analogs (e.g., GpppG); dimethylated cap analog (e.g., m2,7GpppG), a trimethylated cap analog (e.g., m2,2,7GpppG), dimethylated symmetrical cap analogs (e.g., m7Gpppm7G), or anti reverse cap analogs (e.g., ARCA; m7, 2′OmeGpppG, m72′dGpppG, m7,3′OmeGpppG, m7,3′dGpppG and their tetraphosphate derivatives) (See, e.g., Jemielity et al., 2003, RNA 9: 1108-1122). In other embodiments, the 5′-cap may be an ARCA cap (3′-OMe-m7G(5′)pppG) or an mCAP (m7G(5′)ppp(5′)G, N⁷-Methyl-Guanosine-5′-Triphosphate-5′-Guanosine).

### 5′ and 3′ Untranslated Regions (UTRs)

In some embodiments, the polynucleotide comprising an mRNA coding sequence for the HFE protein or a fragment thereof may further comprise a 5′ untranslated region (5′ UTR) and/or a 3′ untranslated region (3′ UTR). As is understood in the art, the 5′ and/or 3′ UTR may affect an mRNA's stability or efficiency of translation. In an exemplary embodiment, the polynucleotide comprising an mRNA coding sequence for the HFE protein or a fragment thereof comprises a 5′ UTR and a 3′ UTR.

Examples of 5′ UTR and 3′ UTR sequences may be found in U.S. Pat. No. 9,149,506 and WO/2018/222890.

In some embodiments, the polynucleotide comprising an mRNA coding sequence for the HFE protein or a fragment thereof may comprise a 5′ UTR that is at least about 25, 50, 75, 100, 125, 150, 175, 200, 300, 400, or 500 nucleotides. In some embodiments, a 5′ UTR contains about 10 to 150 nucleotides (e.g., about 25 to 100 nucleotides, about 35 to 75 nucleotides, about 40 to 60 nucleotides, or about 50 nucleotides). In an exemplary embodiment, the 5′ UTR is about 45, 46, 47, 48, 49, or 50 nucleotides in length.

In some embodiments, the 5′ UTR is derived from an mRNA molecule known in the art to be relatively stable (e.g., histone, tubulin, globin, GAPDH, actin, or citric acid cycle enzymes) to increase the stability of the polynucleotide. In other embodiments, a 5′ UTR sequence may include a partial sequence of a CMV immediate-early 1 (IE1) gene. In some embodiments, the 5′ UTR comprises a sequence selected from the 5′ UTRs of human IL-6, alanine aminotransferase 1, human apolipoprotein E, human fibrinogen alpha chain, human transthyretin, human haptoglobin, human alpha-1-antichymotrypsin, human antithrombin, human alpha-1-antitrypsin, human albumin, human beta globin, human complement C3, human complement C5, SynK, AT1G58420, mouse beta globin, mouse albumin, and a tobacco etch virus, or fragments of any of the foregoing.

In an exemplary embodiment, the 5′ UTR comprises or consists of a sequence set forth in SEQ ID NO: 33. In yet another exemplary embodiment, the 5′ UTR is a fragment of a sequence set forth in SEQ ID NO: 33, such as a fragment of at least 10, 15, 20, 25, 30, 35, 40, or 45 contiguous nucleotides of SEQ ID NO: 33.

In an alternative embodiment, the 5′ UTR is derived from a tobacco etch virus (TEV). In one embodiment, the 5′ UTR comprises or consists of a sequence set forth in SEQ ID NO: 34. In another embodiment, the 5′ UTR is a fragment of a sequence set forth in SEQ ID NO: 34, such as a fragment of at least 10, 20, 30, 40, 50, 60, 70, 80, 90, 100, 110, 120, or 125 contiguous nucleotides of SEQ ID NO: 34.

In some embodiments, the polynucleotide comprising an mRNA coding sequence for the HFE protein or a fragment thereof comprises an internal ribosome entry site (IRES). As is understood in the art, an IRES is an RNA element that allows for translation initiation in an end-independent manner. In exemplary embodiments, the IRES is in the 5′ UTR. In other embodiments, the IRES may be outside the 5′ UTR.

In some embodiments, the polynucleotide comprising an mRNA coding sequence for the HFE protein or a fragment thereof may comprise a 3′ UTR that is at least about 25, 50, 75, 100, 125, 150, 175, 200, 300, 400, or 500 nucleotides. In some embodiments, a 3′ UTR contains about 25 to 200 nucleotides (e.g., about 50 to 150 nucleotides, about 75 to 125 nucleotides, about 80 to 120 nucleotides, or about 100 nucleotides). In an exemplary embodiment, the 3′ UTR is about 100, 101, 102, 103, 104, 105, 106, 107, 108, 109, or 110 nucleotides in length.

In some embodiments, the 3′ UTR comprises a sequence selected from the 3′ UTRs of alanine aminotransferase 1, human apolipoprotein E, human fibrinogen alpha chain, human haptoglobin, human antithrombin, human alpha globin, human beta globin, human complement C3, human growth factor, human hepcidin, MALAT-1, mouse beta globin, mouse albumin, and Xenopus beta globin, or fragments of any of the foregoing.

In an exemplary embodiment, the 3′ UTR comprises or consists of a sequence set forth in SEQ ID NO: 35. In another exemplary embodiment, the 3′ UTR is a fragment of a sequence set forth in SEQ ID NO: 35, such as a fragment of at least 20, 30, 40, 50, 60, 70, 80, 90, or 100 contiguous nucleotides of SEQ ID NO: 35.

In an alternative embodiment, the 3′ UTR is derived from Xenopus beta globin. In one embodiment, the 3′ UTR comprises or consists of a sequence set forth in SEQ ID NO: 36. In another embodiment, the 3′ UTR is a fragment of a sequence set forth in SEQ ID NO: 36, such as a fragment of at least 10, 20, 30, 40, 50, 60, 70, 80, 90, 100, 110, 120, 130, 140, or 150 contiguous nucleotides of SEQ ID NO: 36.

In certain exemplary embodiments, the polynucleotide encoding HFE comprises a 5′ UTR sequence of SEQ ID NO: 33 and a 3′ UTR sequence of SEQ ID NO: 35.

### Tail Region

In some embodiments, the polynucleotide comprising an mRNA coding sequence for the HFE protein or a fragment thereof comprises a tail region, which can serve to protect the mRNA from exonuclease degradation. In some embodiments, the tail region can be a polyA tail.

PolyA tails can be added using a variety of methods known in the art, e.g., using poly(A) polymerase to add tails to synthetic or in vitro transcribed RNA. Other methods include the use of a transcription vector to encode polyA tails or the use of a ligase (e.g., via splint ligation using a T4 RNA ligase and/or T4 DNA ligase), wherein polyA may be ligated to the 3′ end of a sense RNA. In some embodiments, a combination of any of the above methods is utilized.

In some embodiments, the polynucleotide comprising an mRNA coding sequence for the HFE protein or a fragment thereof comprises a 3′ polyA tail structure. In some embodiments, the length of the polyA tail can be at least about 5, 10, 15, 20, 25, 30, 35, 40, 45, 50, 100, 200, or 300 nucleotides. In some embodiments, a 3′ polyA tail contains about 5 to 300 adenosine nucleotides (e.g., about 30 to 250 adenosine nucleotides, about 60 to 220 adenosine nucleotides, about 80 to 200 adenosine nucleotides, about 90 to about 150 adenosine nucleotides, or about 100 to about 120 adenosine nucleotides). In an exemplary embodiment, the 3′ polyA tail is about 80 nucleotides in length. In another exemplary embodiment, the 3′ polyA tail is about 100 nucleotides in length. In yet another exemplary embodiment, the 3′ polyA tail is about 115 nucleotides in length. In yet another exemplary embodiment, the 3′ polyA tail is about 250 nucleotides in length.

In some embodiments, the 3′ polyA tail comprises one or more UNA monomers. In some embodiments, the 3′ polyA tail contains 2, 3, 4, 5, 10, 15, 20, or more UNA monomers. In an exemplary embodiment, the 3′ polyA tail contains 2 UNA monomers. In a further exemplary embodiment, the 3′ polyA tail contains 2 UNA monomers which are found consecutively, i.e., contiguous to each other in the 3′ polyA tail.

In some embodiments, the polynucleotide comprising an mRNA coding sequence for the HFE protein or a fragment thereof comprises a 3′ polyC tail structure. In some embodiments, the length of the polyC tail can be at least about 5, 10, 15, 20, 25, 30, 35, 40, 45, 50, 100, 200, or 300 nucleotides. In some embodiments, a 3′ polyC tail contains about 5 to 300 cytosine nucleotides (e.g., about 30 to 250 cytosine nucleotides, about 60 to 220 cytosine nucleotides, about 80 to about 200 cytosine nucleotides, about 90 to 150 cytosine nucleotides, or about 100 to about 120 cytosine nucleotides). In an exemplary embodiment, the 3′ polyC tail is about 80 nucleotides in length. In another exemplary embodiment, the 3′ polyC tail is about 100 nucleotides in length. In yet another exemplary embodiment, the 3′ polyC tail is about 115 nucleotides in length. In yet another exemplary embodiment, the 3′ polyC tail is about 250 nucleotides in length. The polyC tail may be added to the polyA tail or may substitute the polyA tail. The polyC tail may be added to the 5′ end of the polyA tail or the 3′ end of the polyA tail.

In some embodiments, the length of the polyA and/or polyC tail is adjusted to control the stability of a modified polynucleotide of the invention and, thus, the transcription of protein. For example, since the length of the polyA tail can influence the half-life of a polynucleotide, the length of the polyA tail can be adjusted to modify the level of resistance of the mRNA to nucleases and thereby control the time course of polynucleotide expression and/or polypeptide production in a target cell.

### Triple Stop Codon

In some embodiments, the polynucleotide comprising an mRNA coding sequence for the HFE protein or a fragment thereof may comprise a sequence immediately downstream of the CDS that creates a triple stop codon. The triple stop codon may be incorporated to enhance the efficiency of translation. In some embodiments, the translatable oligomer may comprise the sequence AUAAGUGAA (SEQ ID NO: 65) immediately downstream of a HFE CDS described herein, as exemplified in SEQ ID NOs: 4-31.

### Translation Initiation Sites

In some embodiments, the polynucleotide comprising an mRNA coding sequence for the HFE protein or a fragment thereof may comprise a translation initiation site. Such sequences are known in the art and include the Kozak sequence. See, e.g., Kozak, Marilyn, 1988, Mol. and Cell Biol. 8: 2737-2744; Kozak, Marilyn, 1991, J. Biol. Chem. 266: 19867-19870; Kozak, Marilyn, 1990, PNAS USA 87:8301-8305; and Kozak, Marilyn, 1989, J. Cell Biol. 108: 229-241. As is understood in the art, a Kozak sequence is a short consensus sequence centered around the translational initiation site of eukaryotic mRNAs that allows for efficient initiation of translation of the mRNA. The ribosomal translation machinery recognizes the AUG initiation codon in the context of the Kozak sequence.

In some embodiments, the translation initiation site, e.g., a Kozak sequence, is inserted upstream of the coding sequence for HFE. In some embodiments, the translation initiation site is inserted downstream of a 5′ UTR. In certain exemplary embodiments, the translation initiation site is inserted upstream of the coding sequence for HFE and downstream of a 5′ UTR.

As is understood in the art, the length of the Kozak sequence may vary. Generally, increasing the length of the leader sequence enhances translation.

In some embodiments, the polynucleotide comprising an mRNA coding sequence for the HFE protein or a fragment thereof comprises a Kozak sequence having the sequence of SEQ ID NO: 66. In certain exemplary embodiments, the polynucleotide comprising an mRNA coding sequence for the HFE protein or a fragment thereof comprises a Kozak sequence having the sequence of SEQ ID NO: 66, wherein the Kozak sequence is immediately downstream of a 5′ UTR and immediately upstream of the coding sequence for HFE.

### Synthesis Methods

In various aspects, this invention provides methods for synthesis of polynucleotides comprising an mRNA coding sequence for the HFE protein or a fragment thereof.

Polynucleotides of this invention can be synthesized and isolated using methods disclosed herein, as well as any pertinent techniques known in the art.

Some methods for preparing nucleic acids are given in, for example, Merino, Chemical Synthesis of Nucleoside Analogues, (2013); Gait, Oligonucleotide synthesis: a practical approach (1984); Herdewijn, Oligonucleotide Synthesis, Methods in Molecular Biology, Vol. 288 (2005).

In some embodiments, a polynucleotide comprising an mRNA coding sequence for the HFE protein or a fragment thereof can be made by an in vitro transcription (IVT) reaction. A mix of nucleoside triphosphates (NTP) can be polymerized using T7 reagents, for example, to yield RNA from a DNA template. The DNA template can be degraded with RNase-free DNase, and the RNA column-separated.

In some embodiments, a ligase can be used to link a synthetic oligomer to the 3′ end of an RNA molecule or an RNA transcript to form a polynucleotide of the invention. The synthetic oligomer that is ligated to the 3′ end can provide the functionality of a polyA tail, and advantageously provide resistance to its removal by 3′-exoribonucleases. The ligated product can have increased specific activity and provide increased levels of protein expression.

In certain embodiments, the ligated product can be made with an RNA transcript that has native specificity. The ligated product can be a synthetic molecule that retains the structure of the RNA transcript at the 5′ end to ensure compatibility with the native specificity.

In further embodiments, the ligated product be made with an exogenous RNA transcript or non-natural RNA. The ligated product can be a synthetic molecule that retains the structure of the RNA.

Without wishing to be bound by theory, the canonical mRNA degradation pathway in cells includes the steps: (i) the polyA tail is gradually cut back to a stub by 3′ exonucleases, shutting down the looping interaction required for efficient translation and leaving the cap open to attack; (ii) decapping complexes remove the 5′-cap; (iii) the unprotected and translationally incompetent residuum of the transcript is degraded by 5′ and 3′ exonuclease activity.

Embodiments of this invention involve new polynucleotide structures which can have increased translational activity over a native transcript. Among other things, the polynucleotides provided herein may prevent exonucleases from trimming back the polyA tail in the process of de-adenylation.

### Lipid-Based Formulations

Lipid-based formulations have been increasingly recognized as one of the most promising delivery systems for RNA due to their biocompatibility and their ease of large-scale production. Cationic lipids have been widely studied as synthetic materials for delivery of RNA. After mixing together, nucleic acids are condensed by cationic lipids to form lipid/nucleic acid complexes known as lipoplexes. These lipid complexes are able to protect genetic material from the action of nucleases and to deliver it into cells by interacting with the negatively charged cell membrane. Lipoplexes can be prepared by directly mixing positively charged lipids at physiological pH with negatively charged nucleic acids.

Conventional liposomes consist of a lipid bilayer that can be composed of cationic, anionic, or neutral (phospho)lipids and cholesterol, which encloses an aqueous core. Both the lipid bilayer and the aqueous space can incorporate hydrophobic or hydrophilic compounds, respectively. Liposome characteristics and behavior in vivo can be modified by addition of a hydrophilic polymer coating, e.g., polyethylene glycol (PEG), to the liposome surface to confer steric stabilization. Furthermore, liposomes can be used for specific targeting by attaching ligands (e.g., antibodies, peptides, and carbohydrates) to its surface or to the terminal end of the attached PEG chains.

Liposomes are colloidal lipid-based and surfactant-based delivery systems composed of a phospholipid bilayer surrounding an aqueous compartment. They may present as spherical vesicles and can range in size from 20 nm to a few microns. Cationic lipid-based liposomes are able to complex with negatively charged nucleic acids via electrostatic interactions, resulting in complexes that offer biocompatibility, low toxicity, and the possibility of the large-scale production required for in vivo clinical applications. Liposomes can fuse with the plasma membrane for uptake; once inside the cell, the liposomes are processed via the endocytic pathway and the genetic material is then released from the endosome/carrier into the cytoplasm. Liposomes have long been perceived as drug delivery vehicles because of their superior biocompatibility, given that liposomes are basically analogs of biological membranes, and can be prepared from both natural and synthetic phospholipids.

Cationic liposomes have been traditionally the most commonly used non-viral delivery systems for oligonucleotides, including plasmid DNA, antisense oligos, and siRNA/small hairpin R A-shRNA). Cationic lipids, such as DOTAP, (1,2-dioleoyl-3-trimethylammonium-propane) and DOTMA (N-[1-(2,3-dioleoyloxy)propyl]-N,N,N-trimethyl-ammonium methyl sulfate) can form complexes or lipoplexes with negatively charged nucleic acids to form nanoparticles by electrostatic interaction, providing high in vitro transfection efficiency. Furthermore, neutral lipid-based nanoliposomes for RNA delivery as, e.g., neutral 1,2-dioleoyl-sn-glycero-3-phosphatidylcholine (DOPC)-based nanoliposomes were developed.

According to some embodiments, the polynucleotides described herein that encode HFE are lipid formulated. The lipid formulation is preferably selected from, but not limited to, liposomes, lipoplexes, copolymers, such as PLGA, and lipid nanoparticles. In an exemplary embodiment, the lipid formulation is a lipid nanoparticle. In a further exemplary embodiment, the polynucleotides are encapsulated in a lipid nanoparticle, wherein the lipid nanoparticles are part of a pharmaceutical composition that is free of liposomes.

In one preferred embodiment, a lipid nanoparticle (LNP) comprises:

(a) a nucleic acid (e.g., a polynucleotide encoding HFE),

(b) a cationic lipid,

(c) an aggregation reducing agent (such as polyethylene glycol (PEG) lipid or PEG-modified lipid),

(d) optionally a non-cationic lipid (such as a neutral lipid), and

(e) optionally, a sterol.

In one embodiment, the lipid nanoparticle formulation consists of (i) at least one cationic lipid; (ii) a neutral lipid; (iii) a sterol, e.g., cholesterol; and (iv) a PEG-lipid, in a molar ratio of about 20-60% cationic lipid: 5-25% neutral lipid: 25-55% sterol; 0.5-15% PEG-lipid.

### Thiocarbamate and Carbamate-Containing Lipid Formulations

Some examples of lipids and lipid compositions for delivery of a polynucleotide encoding HFE are given in WO/2015/074085 and U.S. Patent Publication Nos. US 2018/0169268 and US 20180170866. In certain embodiments, the lipid is a compound of the following formula:

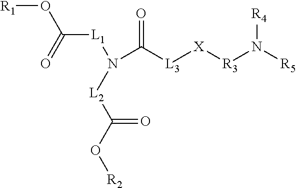

wherein
R₁ and R₂ both consist of a linear alkyl consisting of 1 to 14 carbons, or an alkenyl or alkynyl consisting of 2 to 14 carbons;
L₁ and L₂ both consist of a linear alkylene or alkenylene consisting of 5 to 18 carbons, or forming a heterocycle with N;
X is S;
L₃ consists of a bond or a linear alkylene consisting of 1 to 6 carbons, or forming a heterocycle with N;
R₃ consists of a linear or branched alkylene consisting of 1 to 6 carbons; and
R₄ and R₅ are the same or different, each consisting of a hydrogen or a linear or branched alkyl consisting of 1 to 6 carbons;
   or a pharmaceutically acceptable salt thereof.

The lipid formulation may contain one or more ionizable cationic lipids selected from among the following:

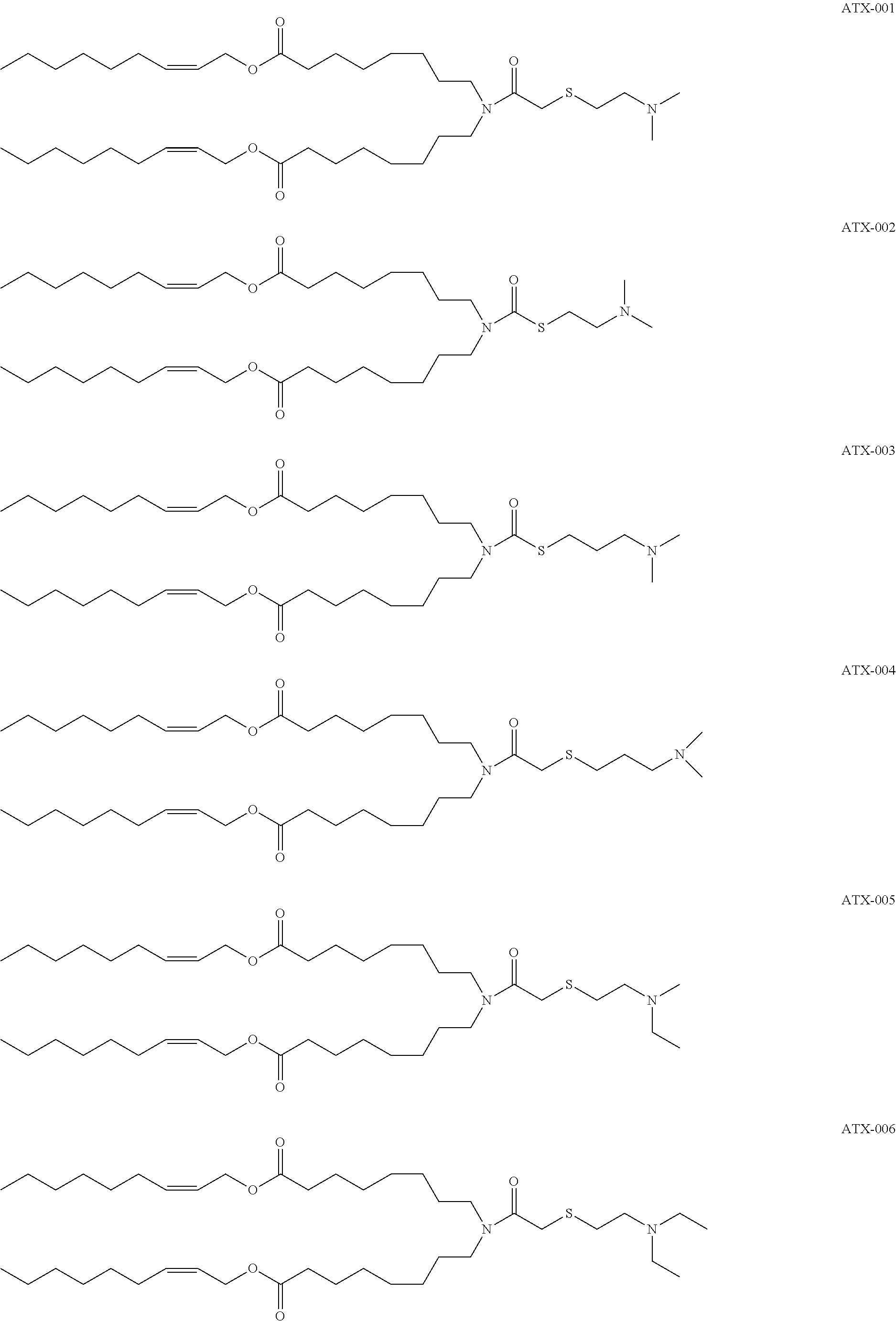

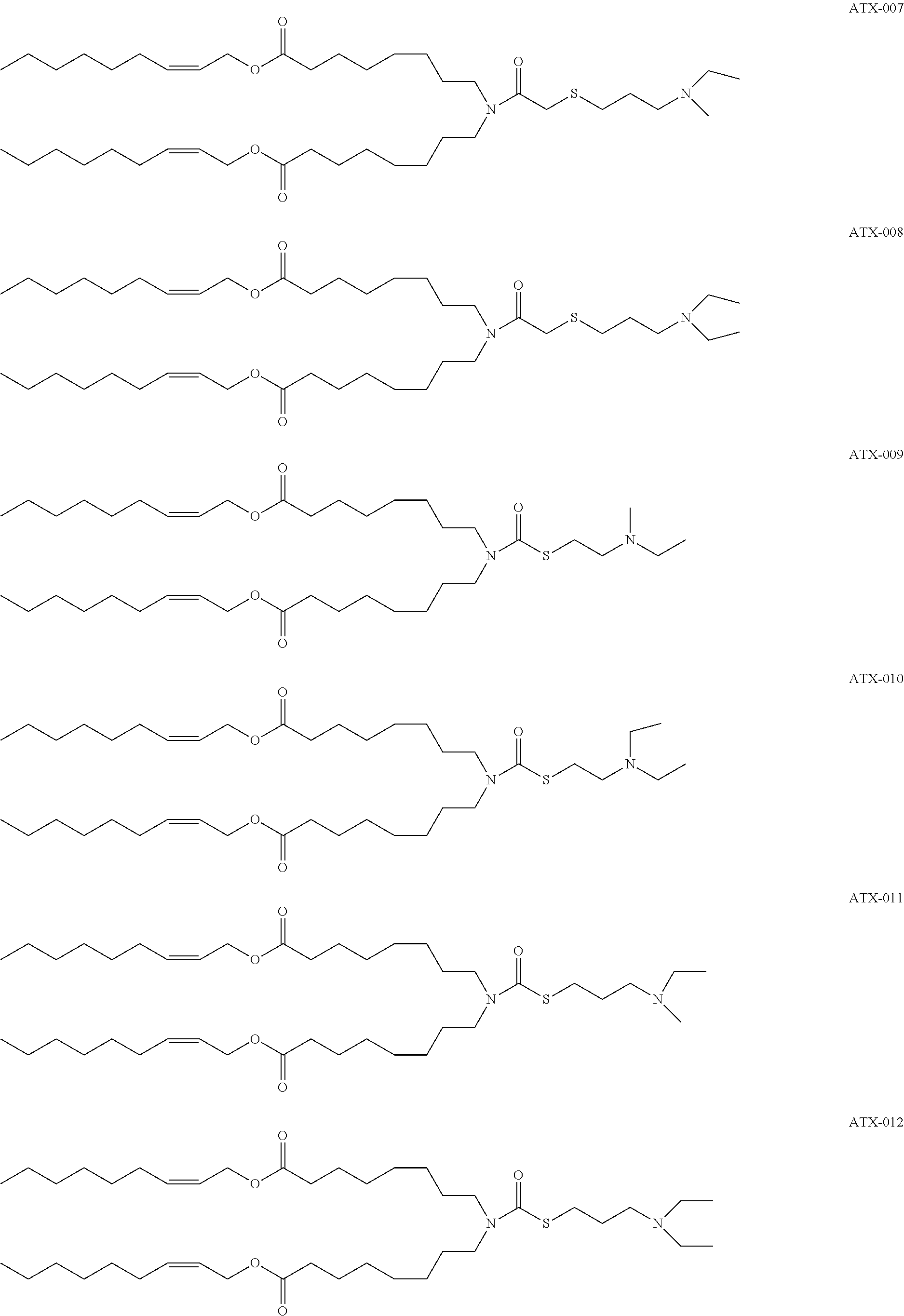

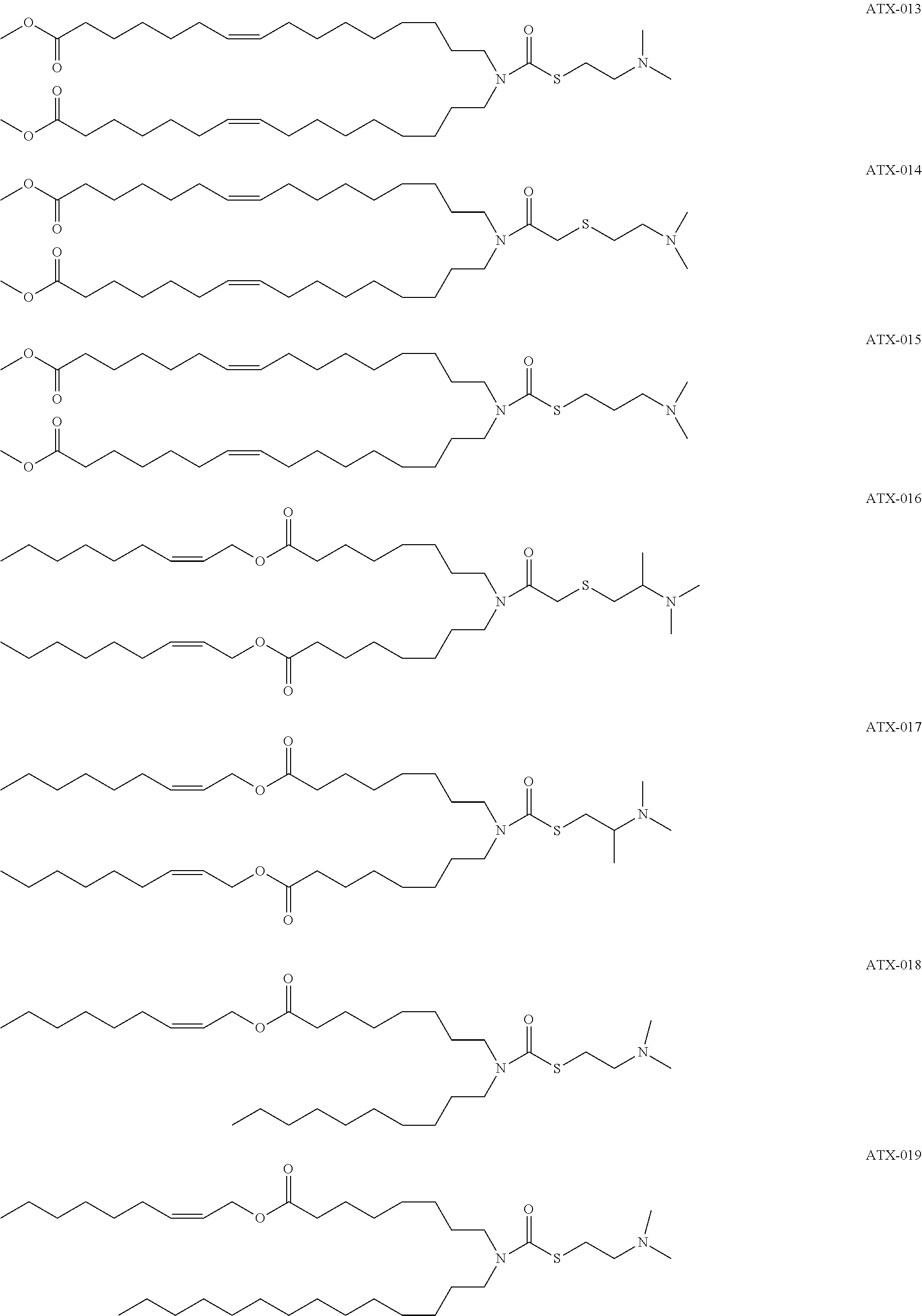

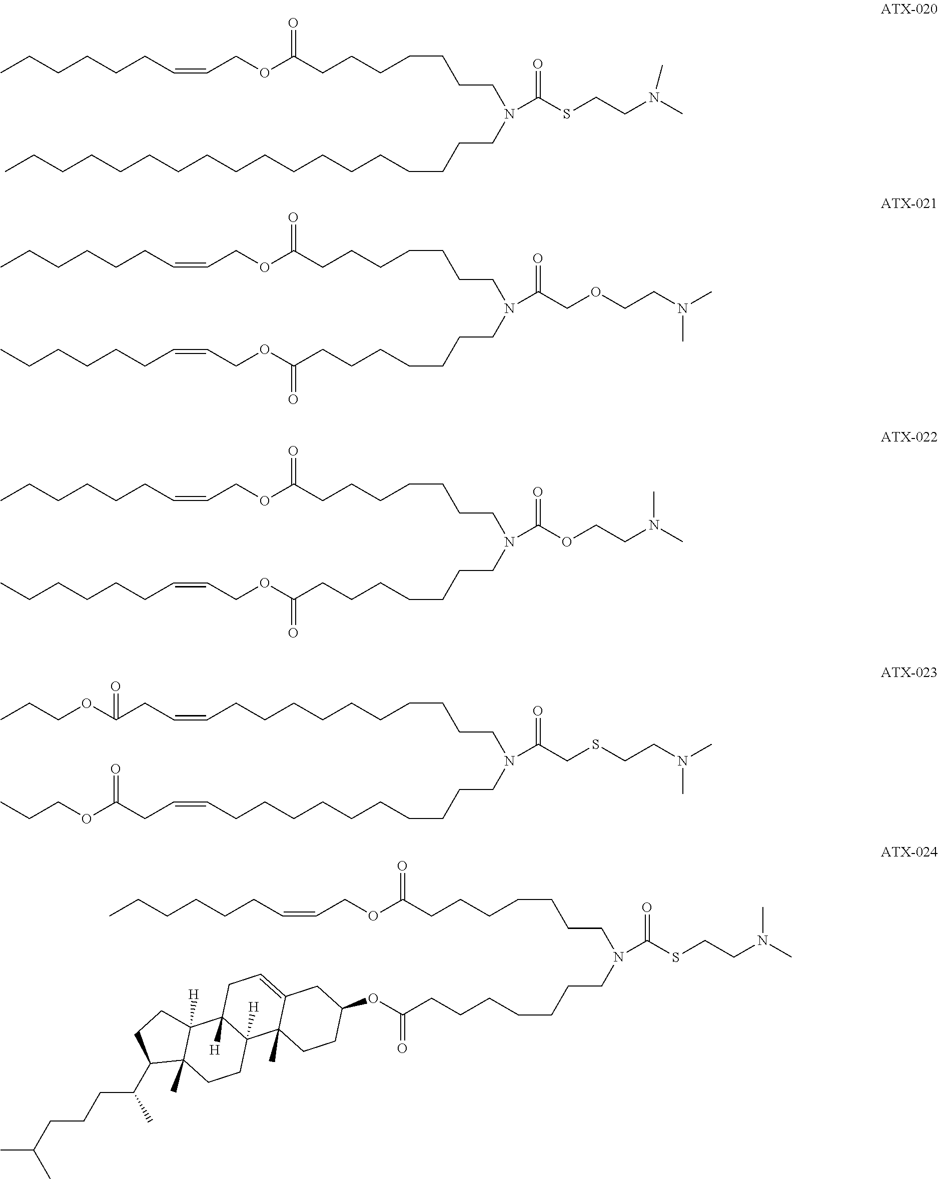

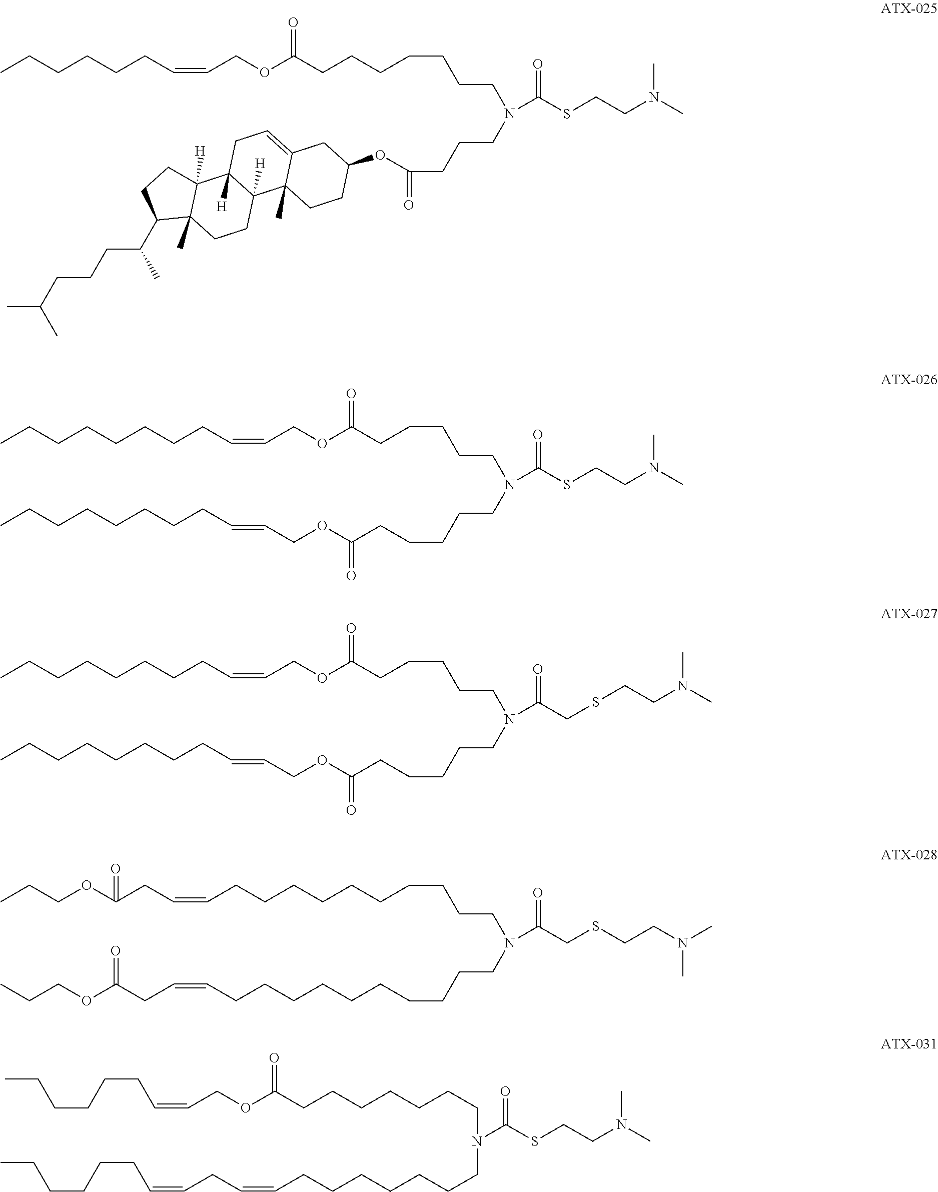

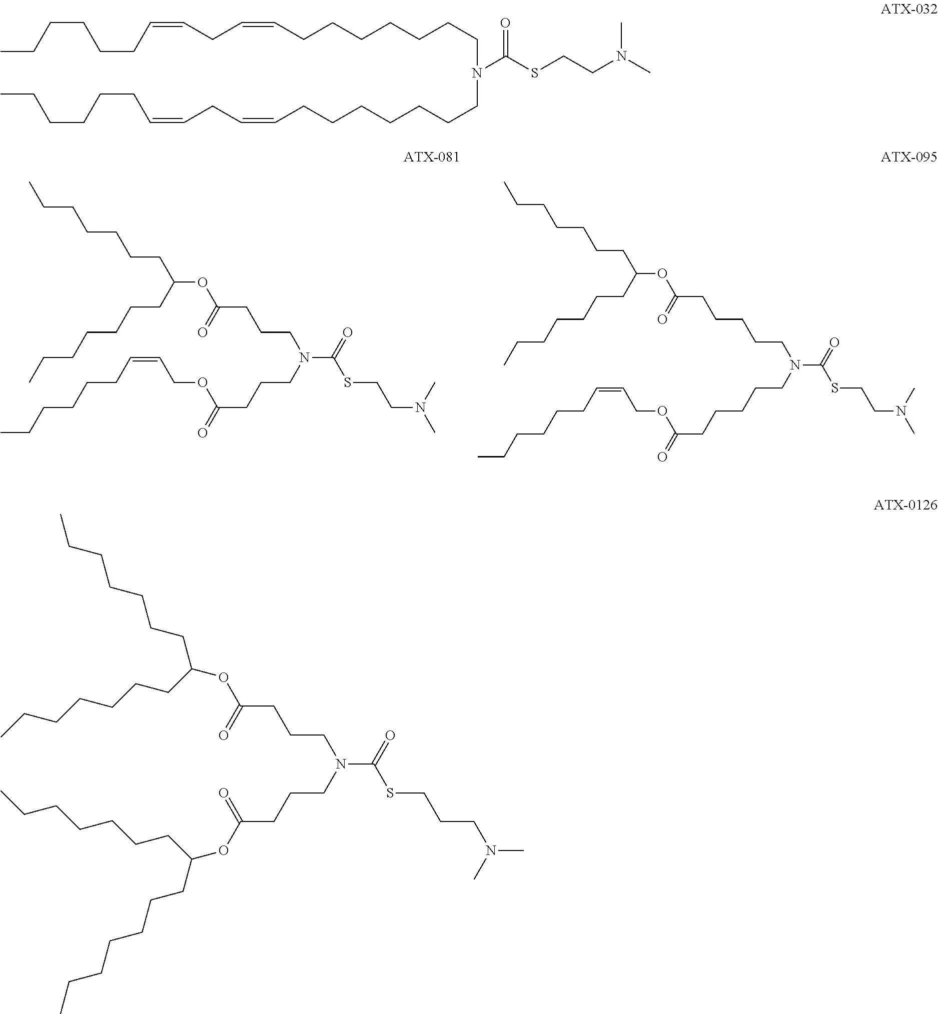

### Cationic Lipids

The lipid nanoparticle (LNP) encapsulating a polynucleotide of the invention preferably includes a cationic lipid suitable for forming a lipid nanoparticle. Preferably, the cationic lipid carries a net positive charge at about physiological pH.

The cationic lipid may be, for example, N,N-dioleyl-N,N-dimethylammonium chloride (DODAC), N,N-distearyl-N,N-dimethylammonium bromide (DDAB), 1,2-dioleoyltrimethylammoniumpropane chloride (DOTAP) (also known as N-(2,3-dioleoyloxy)propyl)-N,N,N-trimethylammonium chloride and 1,2-Dioleyloxy-3-trimethylaminopropane chloride salt), N-(1-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-di-y-linolenyloxy-N,N-dimethylaminopropane (γ-DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.CI), 1,2-Dilinoleoyl-3-trimethylaminopropane chloride salt (DLin-TAP.CI), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DM A), 2,2-Dilinoleyl-4-dimethylaminomethyl[1,3]-dioxolane (DLin-K-DMA) or analogs thereof, (3aR,5s, 6aS)—N,N-dimethyl-2,2-di((9Z,12Z)-octadeca-9,12-dienyl)tetrahydro-3 aH-cyclopenta[d][1,3]dioxol-5-amine, (6Z,9Z,28Z,31Z)-heptatriaconta-6,9,28,31-tetraen-19-yl4-(dimethylamino)butanoate (MC3), 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (C12-200), 2,2-dilinoleyl-4-(2-dimethylaminoethyl)-[1,3]-dioxolane (DLin-K-C2-DMA), 2,2-dilinoleyl-4-dimethylaminomethyl[1,3]-dioxolane (DLin-K-DMA), (6Z,9Z,28Z,31Z)-heptatriaconta-6,9,28 31-tetraen-19-yl 4-(dimethylamino) butanoate (DLin-M-C3-DMA), 3-((6Z,9Z,28Z,31Z)-heptatriaconta-6,9,28,3 1-tetraen-19-yloxy)-N,N-dimethylpropan-1-amine (MC3 Ether), 4-((6Z,9Z,28Z,31 Z)-heptatriaconta-6,9,28,31-tetraen-19-yloxy)-N,N-dimethylbutan-1-amine (MC4 Ether), or any combination of any of the foregoing. Other cationic lipids include, but are not limited to, N,N-di stearyl-N,N-dimethylammonium bromide (DDAB), 3P—(N—(N′,N′-dimethylaminoethane)-carbamoyl)cholesterol (DC-Choi), N-(1-(2,3-dioleyloxy)propyl)-N-2-(sperminecarboxamido)ethyl)-N,N-dimethylammonium trifluoracetate (DOSPA), dioctadecylamidoglycyl carboxyspermine (DOGS), 1,2-dileoyl-sn-3-phosphoethanolamine (DOPE), 1,2-dioleoyl-3-dimethylammonium propane (DODAP), N-(1,2-dimyristyloxyprop-3-yl)-N,N-dimethyl-N-hydroxyethyl ammonium bromide (DMRIE), and 2,2-Dilinoleyl-4-dimethylaminoethyl-[1,3]-dioxolane (XTC). Additionally, commercial preparations of cationic lipids can be used, such as, e.g., LIPOFECTIN (including DOTMA and DOPE, available from GIBCO/BRL), and Lipofectamine (comprising DOSPA and DOPE, available from GIBCO/BRL).

Other suitable cationic lipids are disclosed in International Publication Nos. WO 09/086558, WO 09/127060, WO 10/048536, WO 10/054406, WO 10/088537, WO 10/129709, and WO 2011/153493; U.S. Patent Publication Nos. 2011/0256175, 2012/0128760, and 2012/0027803; U.S. Pat. No. 8,158,601; and Love et al., 2010, PNAS 107(5): 1864-69. Other suitable amino lipids include those having alternative fatty acid groups and other dialkylamino groups, including those, in which the alkyl substituents are different (e.g., N-ethyl-N-methylamino-, and N-propyl-N-ethylamino-). In general, amino lipids having less saturated acyl chains are more easily sized, particularly when the complexes must be sized below about 0.3 microns, for purposes of filter sterilization. Amino lipids containing unsaturated fatty acids with carbon chain lengths in the range of C14 to C22 may be used. Other scaffolds can also be used to separate the amino group and the fatty acid or fatty alkyl portion of the amino lipid.

In certain embodiments, amino or cationic lipids of the invention have at least one protonatable or deprotonatable group, such that the lipid is positively charged at a pH at or below physiological pH (e.g., pH 7.4), and neutral at a second pH, preferably at or above physiological pH. It will, of course, be understood that the addition or removal of protons as a function of pH is an equilibrium process, and that the reference to a charged or a neutral lipid refers to the nature of the predominant species and does not require that all of the lipid be present in the charged or neutral form. Lipids that have more than one protonatable or deprotonatable group, or which are zwitterionic, are not excluded from use in the invention. In certain embodiments, the protonatable lipids have a pKa of the protonatable group in the range of about 4 to about 11, e.g., a pKa of about 5 to about 7.

The cationic lipid can comprise from about 20 mol % to about 70 mol % or 75 mol % or from about 45 mol % to about 65 mol % or about 20, 25, 30, 35, 40, 45, 50, 55, 60, 65, or about 70 mol % of the total lipid present in the particle. In another embodiment, the lipid nanoparticles include from about 25% to about 75% on a molar basis of cationic lipid, e.g., from about 20% to about 70%, from about 35% to about 65%, from about 45% to about 65%, about 60%, about 57.5%, about 57.1%, about 50% or about 40% on a molar basis (based upon 100% total moles of lipid in the lipid nanoparticle). In one embodiment, the ratio of cationic lipid to nucleic acid is from about 3 to about 15, such as from about 5 to about 13 or from about 7 to about 11.

### Pharmaceutical Compositions

In some aspects, this application provides pharmaceutical compositions containing a polynucleotide of the invention capable of encoding a functionally active HFE protein or functional fragment thereof and a pharmaceutically acceptable carrier.

A pharmaceutical composition can be capable of local or systemic administration. In some aspects, a pharmaceutical composition can be capable of any mode of administration. In certain aspects, the administration can be by any route, including intravenous, subcutaneous, pulmonary, intramuscular, intraperitoneal, dermal, oral, inhalation or nasal administration.

Embodiments of this invention include pharmaceutical compositions containing an HFE-encoding polynucleotide in a lipid formulation, e.g., a lipid nanoparticle (LNP).

In some embodiments, a pharmaceutical composition may comprise one or more lipids selected from cationic lipids, anionic lipids, sterols, pegylated lipids, and any combination of the foregoing. In some embodiments, the pharmaceutical composition containing an HFE-encoding polynucleotide comprises a cationic lipid, a phospholipid, cholesterol, and a pegylated lipid.

In certain exemplary embodiments, a pharmaceutical composition of the invention is free of liposomes.

In further embodiments, a pharmaceutical composition can include nanoparticles.

In certain exemplary embodiments, a pharmaceutical composition of the invention comprises an HFE-encoding polynucleotide of the invention encapsulated in lipid nanoparticles (LNPs) and is free of liposomes.

Some examples of lipids and lipid compositions for delivery of an HFE-encoding polynucleotide of this invention are given in WO/2015/074085, which is hereby incorporated by reference in its entirety. In certain embodiments, the lipid is a cationic lipid. In some embodiment, the cationic lipid comprises a compound of formula II:

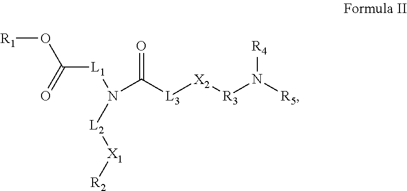

in which R₁ and R₂ are the same or different, each a linear or branched alkyl, alkenyl, or alkynyl, L₁ and L₂ are the same or different, each a linear alkyl having at least five carbon atoms, or form a heterocycle with the N, X₁ is a bond, or is whereby L₂-CO—O—R₂ is formed X₂ is S or O, L₃ is a bond or a lower alkyl, R₃ is a lower alkyl, R₄ and R₅ are the same or different, each a lower alkyl. What is also described herein is the compound of formula II, in which L₃ is absent, R₁ and R₂ each consists of at least seven carbon atoms, R₃ is ethylene or n-propylene, R₄ and R₅ are methyl or ethyl, and L₁ and L₂ each consists of a linear alkyl having at least five carbon atoms. What is also described herein is the compound of formula II, in which L₃ is absent, R₁ and R₂ each consists of at least seven carbon atoms, R₃ is ethylene or n-propylene, R₄ and R₅ are methyl or ethyl, and L₁ and L₂ each consists of a linear alkyl having at least five carbon atoms. What is also described herein is the compound of formula II, in which L₃ is absent, R₁ and R₂ each consists of an alkenyl of at least nine carbon atoms, R₃ is ethylene or n-propylene, R₄ and R₅ are methyl or ethyl, and L₁ and L₂ each consists of a linear alkyl having at least five carbon atoms. What is also described herein is the compound of formula II, in which L₃ is methylene, R₁ and R₂ each consists of at least seven carbon atoms, R₃ is ethylene or n-propylene, R₄ and R₅ are methyl or ethyl, and L₁ and L₂ each consists of a linear alkyl having at least five carbon atoms. What is also described herein is the compound of formula II, in which L₃ is methylene, R₁ and R₂ each consists of at least nine carbon atoms, R₃ is ethylene or n-propylene, R₄ and R₅ are each methyl, L₁ and L₂ each consists of a linear alkyl having at least seven carbon atoms. What is also described herein is the compound of formula II, in which L₃ is methylene, R₁ consists of an alkenyl having at least nine carbon atoms and R₂ consists of an alkenyl having at least seven carbon atoms, R₃ is n-propylene, R₄ and R₅ are each methyl, L₁ and L₂ each consists of a linear alkyl having at least seven carbon atoms. What is also described herein is the compound of formula II, in which L₃ is methylene, R₁ and R₂ each consists of an alkenyl having at least nine carbon atoms, R₃ is ethylene, R₄ and R₅ are each methyl, L₁ and L₂ each consists of a linear alkyl having at least seven carbon atoms.

In exemplary embodiments, the cationic lipid comprises a compound of selected from the group consisting of ATX-001, ATX-002, ATX-003, ATX-004, ATX-005, ATX-006, ATX-007, ATX-008, ATX-009, ATX-010, ATX-011, ATX-012, ATX-013, ATX-014, ATX-015, ATX-016, ATX-017, ATX-018, ATX-019, ATX-020, ATX-021, ATX-022, ATX-023, ATX-024, ATX-025, ATX-026, ATX-027, ATX-028, ATX-029, ATX-030, ATX-031, ATX-032, ATX-081, ATX-095, and ATX-126, or a pharmaceutically acceptable salt thereof.

In exemplary embodiments, the cationic lipid is selected from ATX-002, ATX-081, ATX-095, or ATX-126.

In some embodiments, the cationic lipid or a pharmaceutically acceptable salt thereof, may be presented in a lipid composition, comprising a nanoparticle or a bilayer of lipid molecules. The lipid bilayer preferably further comprises a neutral lipid or a polymer. The lipid composition preferably comprises a liquid medium. The composition preferably further encapsulates a polynucleotide comprising an HFE coding sequence of the present invention. The lipid composition preferably further comprises a polynucleotide of the present invention and a neutral lipid or a polymer. The lipid composition preferably encapsulates the polynucleotide comprising an HFE coding sequence.

In further embodiments, the cationic lipid comprises a compound of formula

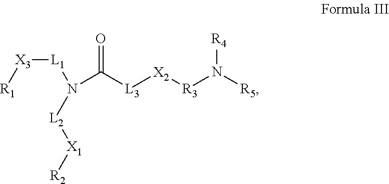

wherein R₁ and R₂ are the same or different, each a linear or branched alkyl consisting of 1 to 9 carbons, an alkenyl or alkynyl consisting of 2 to 11 carbons, or cholesteryl, L₁ and L₂ are the same or different, each a linear alkylene or alkenylene consisting of 5 to 18 carbons, X₁ is whereby -L₂-CO—O—R₂ is formed, X₂ is S or O, X₃ is whereby -L₁-CO—O—R₁ is formed, L₃ is a bond, R₃ is a linear or branched alkylene consisting of 1 to 6 carbons, and R₄ and R₅ are the same or different, each hydrogen or a linear or branched alkyl consisting of 1 to 6 carbons; or a pharmaceutically acceptable salt thereof. In one embodiment, X₂ is S. In another embodiment, R₃ is selected from ethylene, n-propylene, or isobutylene. In yet another embodiment, R₄ and R₅ are separately methyl, ethyl, or isopropyl. In yet another embodiment, L₁ and L₂ are the same. In yet another embodiment, L₁ and L₂ differ. In yet another embodiment, L₁ or L₂ consists of a linear alkylene having seven carbons. In yet another embodiment, L₁ or L₂ consists of a linear alkylene having nine carbons. In yet another embodiment, R₁ and R₂ are the same. In yet another embodiment, R₁ and R₂ differ. In yet another embodiment, R₁ and R₂ each consists of an alkenyl. In yet another embodiment, R₁ and R₂ each consists of an alkyl. In yet another embodiment, the alkenyl consists of a single double bond. In yet another embodiment, R₁ or R₂ consists of nine carbons. In yet another embodiment, R₁ or R₂ consists of eleven carbons. In yet another embodiment, R₁ or R₂ consists of seven carbons. In yet another embodiment, L₃ is a bond, R₃ is ethylene, X₂ is S, and R₄ and R₅ are each methyl. In yet another embodiment, L₃ is a bond, R₃ is n-propylene, X₂ is S, R₄ and R₅ are each methyl. In yet another embodiment, L₃ is a bond, R₃ is ethylene, X₂ is S, and R₄ and R₅ are each ethyl.

As would be appreciated by the skilled artisan, the compounds of formulas II and III form salts that are also within the scope of this disclosure. Reference to a compound of formulas II and III herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of formula II or III contains both a basic moiety, such as, but not limited to, a pyridine or imidazole, and an acidic moiety, such as, but not limited to, a carboxylic acid, zwitterions (“inner salts”) may be formed and are included within the term “salt(s)” as used herein. The salts can be pharmaceutically acceptable (i.e., non-toxic, physiologically acceptable) salts, although other salts are also useful. Salts of the compounds of the formula II or III may be formed, for example, by reacting a compound of formula II or III with an amount of acid or base, such as an equivalent amount, in a medium such as one in which the salt precipitates or in an aqueous medium followed by lyophilization.

Exemplary acid addition salts include acetates, adipates, alginates, ascorbates, aspartates, benzoates, benzenesulfonates, bisulfates, borates, butyrates, citrates, camphorates, camphorsulfonates, cyclopentanepropionates, digluconates, dodecyl sulfates, ethanesulfonates, fumarates, glucoheptanoates, glycerophosphates, hemi sulfates, heptanoates, hexanoates, hydrochlorides, hydrobromides, hydroiodides, 2-hydroxyethanesulfonates, lactates, maleates, methanesulfonates, 2-napthalenesulfonates, nicotinates, nitrates, oxalates, pectinates, persulfates, 3-phenylpropionates, phosphates, picrates, pivalates, propionates, salicylates, succinates, sulfates, sulfonates (such as those mentioned herein), tartarates, thiocyanates, toluenesulfonates (also known as tosylates) undecanoates, and the like. Additionally, acids which are generally considered suitable for the formation of pharmaceutically useful salts from basic pharmaceutical compounds are discussed, for example, by Berge et al., 1977, J. Pharmaceutical Sciences 66(1) 1-19; P. Gould, 1986, International J. Pharmaceutics 33 201-217; Anderson et al., 1996, The Practice of Medicinal Chemistry Academic Press, New York; and in The Orange Book (Food & Drug Administration, Washington, D.C.).

Exemplary basic salts include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines (formed with N,N-bis(dehydroabietyl)ethylenediamine), N-methyl-D-glucamines, N-methyl-D-glucamides, t-butyl amines, and salts with amino acids such as arginine, lysine, and the like. Basic nitrogen-containing groups may be quarternized with agents such as lower alkyl halides (e.g., methyl, ethyl, propyl, and butyl chlorides, bromides, and iodides), dialkyl sulfates (e g, dimethyl, diethyl, dibutyl, and diamyl sulfates), long chain halides (e.g., decyl, lauryl, myristyl, and stearyl chlorides, bromides, and iodides), arylalkyl halides (e.g., benzyl and phenethyl bromides), and others.

All such acid and base salts are intended to be pharmaceutically acceptable salts within the scope of the disclosure and all acid and base salts are considered equivalent to the free forms of the corresponding compounds for purposes of the disclosure. Compounds of formula II or III can exist in unsolvated and solvated forms, including hydrated forms. In general, the solvated forms, with pharmaceutically acceptable solvents such as water, ethanol, and the like, are equivalent to the unsolvated forms for the purposes of this disclosure. Compounds of formula II or III and salts, solvates thereof, may exist in their tautomeric form (for example, as an amide or imino ether). All such tautomeric forms are contemplated herein as part of the present disclosure.

The cationic lipid compounds described herein may be combined with a polynucleotide encoding HFE to form microparticles, nanoparticles, liposomes, or micelles. The polynucleotide of the invention to be delivered by the particles, liposomes, or micelles may be in the form of a gas, liquid, or solid. The cationic lipid compound and the polynucleotide may be combined with other cationic lipid compounds, polymers (synthetic or natural), surfactants, cholesterol, carbohydrates, proteins, lipids, etc. to form the particles. These particles may then optionally be combined with a pharmaceutical excipient to form a pharmaceutical composition.

In certain embodiments, the cationic lipid compounds are relatively non-cytotoxic. The cationic lipid compounds may be biocompatible and biodegradable. The cationic lipid may have a pKa in the range of approximately 5.5 to approximately 7.5, more preferably between approximately 6.0 and approximately 7.0. It may be designed to have a desired pKa between approximately 3.0 and approximately 9.0, or between approximately 5.0 and approximately 8.0.

A composition containing a cationic lipid compound may be 30-70% cationic lipid compound, 0-60% cholesterol, 0-30% phospholipid and 1-10% polyethylene glycol (PEG). Preferably, the composition is 30-40% cationic lipid compound, 40-50% cholesterol, and 10-20% PEG. In other preferred embodiments, the composition is 50-75% cationic lipid compound, 20-40% cholesterol, and 5 to 10% phospholipid, and 1-10% PEG. The composition may contain 60-70% cationic lipid compound, 25-35% cholesterol, and 5-10% PEG. The composition may contain up to 90% cationic lipid compound and 2 to 15% helper lipid. The formulation may be a lipid particle formulation, for example containing 8-30% compound, 5-30% helper lipid, and 0-20% cholesterol; 4-25% cationic lipid, 4-25% helper lipid, 2 to 25% cholesterol, 10 to 35% cholesterol-PEG, and 5% cholesterol-amine; or 2-30% cationic lipid, 2-30% helper lipid, 1 to 15% cholesterol, 2 to 35% cholesterol-PEG, and 1-20% cholesterol-amine; or up to 90% cationic lipid and 2-10% helper lipids, or even 100% cationic lipid.

In some embodiments, the one or more cholesterol-based lipids are selected from cholesterol, PEGylated cholesterol and DC-Chol (N,N-dimethyl-N-ethylcarboxamidocholesterol), and 1,4-bis(3-N-oleylamino-propyl)piperazine. In an exemplary embodiment, the cholesterol-based lipid is cholesterol.

In some embodiments, the one or more pegylated lipids, i.e., PEG-modified lipids. In some embodiments, the one or more PEG-modified lipids comprise a poly(ethylene) glycol chain of up to 5 kDa in length covalently attached to a lipid with alkyl chain(s) of C₆-C₂₀ length. In some embodiments, a PEG-modified lipid is a derivatized ceramide such as N-Octanoyl-Sphingosine-1-[Succinyl(Methoxy Polyethylene Glycol)-2000]. In some embodiments, a PEG-modified or PEGylated lipid is PEGylated cholesterol or Dimyristoylglycerol (DMG)-PEG-2K. In an exemplary embodiment, the PEG-modified lipid is PEGylated cholesterol.

In additional embodiments, a pharmaceutical composition can contain a HFE-encoding polynucleotide of the invention (e.g., a polynucleotide comprising a sequence selected from SEQ ID NO: 4-31) within a viral or bacterial vector.

A pharmaceutical composition of this disclosure may include carriers, diluents or excipients as are known in the art. Examples of pharmaceutical compositions and methods are described, for example, in Remington's Pharmaceutical Sciences, Mack Publishing Co. (A. R. Gennaro ed. 1985), and Remington, The Science and Practice of Pharmacy, 21st Edition (2005).

Examples of excipients for a pharmaceutical composition include antioxidants, suspending agents, dispersing agents, preservatives, buffering agents, tonicity agents, and surfactants.

An effective dose of an agent or pharmaceutical formulation of this invention can be an amount that is sufficient to cause translation of an FIFE-encoding polynucleotide in a cell.

A therapeutically effective dose can be an amount of an agent or formulation that is sufficient to cause a therapeutic effect. A therapeutically effective dose can be administered in one or more separate administrations, and by different routes. As will be appreciated in the art, a therapeutically effective dose or a therapeutically effective amount is largely determined based on the total amount of the therapeutic agent contained in the pharmaceutical compositions of the present invention. Generally, a therapeutically effective amount is sufficient to achieve a meaningful benefit to the subject (e.g., treating, modulating, curing, preventing and/or ameliorating hemochromatosis). For example, a therapeutically effective amount may be an amount sufficient to achieve a desired therapeutic and/or prophylactic effect. Generally, the amount of a therapeutic agent (e.g., a polynucleotide encoding HFE or a functionally active fragment thereof) administered to a subject in need thereof will depend upon the characteristics of the subject. Such characteristics include the condition, disease severity, general health, age, sex and body weight of the subject. One of ordinary skill in the art will be readily able to determine appropriate dosages depending on these and other related factors. In addition, both objective and subjective assays may optionally be employed to identify optimal dosage ranges.

Methods provided herein contemplate single as well as multiple administrations of a therapeutically effective amount of the polynucleotide (e.g., a polynucleotide encoding HFE or a functionally active fragment thereof) described herein. Pharmaceutical compositions comprising a polynucleotide encoding HFE can be administered at regular intervals, depending on the nature, severity and extent of the subject's condition (e.g., the severity of a subject's hemochromatosis disease state and the associated symptoms of hemochromatosis, and/or the subject's HFE activity levels). In some embodiments, a therapeutically effective amount of the polynucleotide (e.g., a polynucleotide encoding HFE or a fragment thereof) of the present invention may be administered periodically at regular intervals (e.g., once every year, once every six months, once every four months, once every three months, once every two months, once a month), once every two weeks, weekly, daily, twice a day, three times a day, four times a day, five times a day, six times a day, or continuously. In an exemplary embodiment, a therapeutically effective amount of the polynucleotide (e.g., a polynucleotide encoding HFE or a fragment thereof) of the present invention is administered weekly, once every two weeks, or monthly.

In some embodiments, the pharmaceutical compositions of the present invention are formulated such that they are suitable for extended-release of the polynucleotide encoding HFE contained therein. Such extended-release compositions may be conveniently administered to a subject at extended dosing intervals. For instance, in one embodiment, the pharmaceutical compositions of the present invention are administered to a subject twice a day, daily or every other day. In some embodiments, the pharmaceutical compositions of the present invention are administered to a subject twice a week, once a week, every 10 days, every two weeks, every 28 days, every month, every six weeks, every eight weeks, every other month, every three months, every four months, every six months, every nine months or once a year. Also contemplated herein are pharmaceutical compositions which are formulated for depot administration (e.g., subcutaneously, intramuscularly) to either deliver or release a polynucleotide encoding HFE over extended periods of time. Preferably, the extended-release means employed are combined with modifications made to the polynucleotide encoding HFE to enhance stability.

In some embodiments, a therapeutically effective dose, upon administration, can result in serum or plasma levels of functional HFE of 1-1000 pg/ml, or 1-1000 ng/ml, or 1-1000 μg/ml, or more.

In some embodiments, administering a therapeutically effective dose of a composition comprising a polynucleotide of the invention can result in increased levels of functional HFE protein in the liver of a treated subject. In some embodiments, administering a composition comprising a polynucleotide of the invention results in a 5%, 10%, 20%, 30%, 40%, 50%, 60%, 70%, 80%, 90%, or 95% increase in levels of functional HFE protein in the liver relative to a baseline functional HFE protein level in the subject prior to treatment. In certain embodiments, administering a therapeutically effective dose of a composition comprising a polynucleotide of the invention will result an increase in levels of functional HFE protein relative to baseline functional HEF levels in the liver of the subject prior to treatment. In some embodiments, the increase in functional HFE levels in the liver relative to baseline functional HFE levels in the liver will be at least 5%, 10%, 20%, 30%, 40%, 50%, 100%, 200%, or more.

In some embodiments, a therapeutically effective dose, when administered regularly, results in increased expression of functional HFE levels in the liver as compared to baseline levels prior to treatment. In some embodiments, administering a therapeutically effective dose of a composition comprising a polynucleotide of the invention results in the expression of a functional HFE protein level at or above about 10 ng/mg, about 20 ng/mg, about 50 ng/mg, about 100 ng/mg, about 150 ng/mg, about 200 ng/mg, about 250 ng/mg, about 300 ng/mg, about 350 ng/mg, about 400 ng/mg, about 450 ng/mg, about 500 ng/mg, about 600 ng/mg, about 700 ng/mg, about 800 ng/mg, about 900 ng/mg, about 1000 ng/mg, about 1200 ng/mg or about 1500 ng/mg of the total protein in the liver of a treated subject.

In some embodiments, administering a therapeutically effective dose of a composition comprising a polynucleotide encoding HFE will result in increased hepcidin mRNA expression, increased plasma hepcidin levels, reduced serum ferritin, reduced plasma iron, reduced urinary iron, and/or reduced liver iron.

In some embodiments, a therapeutically effective dose, when administered regularly, results in a reduction of ferritin levels in a biological sample. In some embodiments, administering a therapeutically effective dose of a composition comprising a polynucleotide encoding HFE results in a reduction of ferritin levels in a biological sample (e.g., a serum sample) by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% as compared to baseline ferritin levels before treatment. In an exemplary embodiment, the biological sample is a serum sample.

In some embodiments, a therapeutically effective dose, when administered regularly, is capable of reducing serum ferritin from levels of greater than 500 μg/L, 600 μg/L, 700 μg/L, 800 μg/L, 900 μg/L, 1000 μg/L, 2000 μg/L, 3000 μg/L, 4000 μg/L, 5000 μg/L, or more to levels of less than 500 μg/L, 400 μg/L, 300 μg/L, 200 μg/L, 100 μg/L, or 50 μg/L. In an exemplary embodiment, a therapeutically effective dose, when administered regularly, is capable of reducing serum ferritin from levels of greater than 1000 μg/L to levels less than 200 μg/L. In a further exemplary embodiment, a therapeutically effective dose, when administered regularly, is capable of reducing serum ferritin from levels of greater than 1000 μg/L to levels less than 50 μg/L.

Measurements of serum ferritin levels can be made using any method known in the art. For instance, serum ferritin can be measured using immunoassays, e.g., enzyme-linked immunosorbent assay (ELISA), immunothemiluminescence (the Abbott Architect assay, the ADVIA Centaur assay, or the Roche ECLIA assay) or an immunoturbidometric assay (Tinta-quant assay). See, e.g., Cullis et al., 2018, British Journal of Haematology. 181(3): 331-340.

In some embodiments, a therapeutically effective dose, when administered regularly, results in a reduction of iron levels in a biological sample. In some embodiments, administering a therapeutically effective dose of a composition comprising a polynucleotide encoding HFE results in a reduction of iron levels in a biological sample (e.g., a plasma or urine sample) by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% as compared to baseline iron levels before treatment. In an exemplary embodiment, the biological sample is a plasma sample. In another exemplary embodiment, the biological sample is a urine sample.

In some embodiments, a therapeutically effective dose, when administered regularly, is capable of reducing plasma iron from levels of greater than 180 μg/dL, 190 μg/dL, 200 μg/dL, 210 μg/dL, 220 μg/dL, 230 μg/dL, 240 μg/dL, 250 μg/dL, 260 μg/dL, 270 μg/dL, or more to levels of less than 180 μg/dL, 150 μg/dL, 125 μg/dL, 100 μg/dL, or 75 μg/dL. In an exemplary embodiment, a therapeutically effective dose, when administered regularly, is capable of reducing plasma iron from levels of greater than 180 μg/dL to levels less than 150 μg/dL. In a further exemplary embodiment, a therapeutically effective dose, when administered regularly, is capable of reducing plasma from levels of greater than 180 μg/dL to levels less than 100 μg/dL.

In further embodiments, a therapeutically effective dose, when administered regularly, increases plasma hepcidin levels in a treated subject. In some embodiments, a therapeutically effective dose, when administered regularly, reduces or eliminates the need for phlebotomy.

A therapeutically effective dose of an active agent (e.g., a composition comprising a polynucleotide encoding HFE) in vivo can be a dose of about 0.001 to about 500 mg/kg body weight. For instance, the therapeutically effective dose may be about 0.001-0.01 mg/kg body weight, or 0.01-0.1 mg/kg, or 0.1-1 mg/kg, or 1-10 mg/kg, or 10-100 mg/kg. In some embodiments, a composition comprising a polynucleotide encoding HFE is provided at a dose ranging from about 0.1 to about 10 mg/kg body weight, e.g., from about 0.3 to about 5 mg/kg, from about 0.5 to about 4.5 mg/kg, or from about 2 to about 4 mg/kg.

A therapeutically effective dose of an active agent (e.g., a composition comprising a polynucleotide encoding HFE) in vivo can be a dose of at least about 0.001 mg/kg body weight, or at least about 0.01 mg/kg, or at least about 0.1 mg/kg, or at least about 1 mg/kg, or at least about 2 mg/kg, or at least about 3 mg/kg, or at least about 4 mg/kg, or at least about 5 mg/kg, at least about 10 mg/kg, at least about 20 mg/kg, at least about 50 mg/kg, or more. In some embodiments, a composition comprising a polynucleotide encoding HFE is provided at a dose of about 0.1 mg/kg, about 0.5 mg/kg, about 1 mg/kg, about 1.5 mg/kg, about 2 mg/kg, about 2.5 mg/kg, about 3 mg/kg, about 3.5 mg/kg, about 4 mg/kg, about 5 mg/kg, or about 6, 7, 8, 9, 10, 15, 20, 25, 50, 75, or 100 mg/kg. In an exemplary embodiment, a composition comprising a polynucleotide encoding HFE is provided at a dose of about 0.3 mg/kg. In another exemplary embodiment, a composition comprising a polynucleotide encoding HFE is provided at a dose of about 1 mg/kg. In yet another exemplary embodiment, a composition comprising a polynucleotide encoding HFE is provided at a dose of about 3 mg/kg.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein. All of the features disclosed in this specification may be combined in any combination. Each feature disclosed in this specification may be replaced by an alternative feature serving the same, equivalent, or similar purpose.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including” is intended merely to illustrate better the present invention and does not pose a limitation on the scope of the invention unless claimed. No language in the specification should be construed as indicating any non-claimed element as essential to the practice of the present invention.

It is understood that this invention is not limited to the particular methodology, protocols, materials, and reagents described, as these may vary. It is also to be understood that the terminology used herein is for the purpose of describing particular embodiments only, and is not intended to limit the scope of the present invention, which will be encompassed by the appended claims.

### EXAMPLES

The invention now being generally described, will be more readily understood by reference to the following examples, which are included merely for purposes of illustration of certain aspects and embodiments of the present invention, and is not intended to limit the invention.

### Example 1: Expression of HFE Protein in Hepatocytes from HFE mRNA

This example demonstrates that exogenous HFE protein can be produced in cultured human primary hepatocytes following transfection of mRNA using a commercially available delivery agent.

Human primary hepatocytes were purchased (Thermo-Fisher Scientific), cryo-recovered, and plated according to the vendor's recommended procedure. Codon-optimized HFE-coding mRNA (modified to replace uridines with either N1-methyl-pseudouridine [“N1MPU” ] or 5-methoxyuridine [“5MOU”]) was transfected using Lipofectamine MessengerMAX (Thermo-Fisher Scientific) at varying amounts. The RNA sequence used in this example is illustrated in SEQ ID NO: 67, which comprises a 5′-cap (providing a single 5′ A residue), a 5′ UTR of SEQ ID NO: 33, an HFE coding sequence of SEQ ID NO: 4 (encoding the protein of SEQ ID NO: 32), and a 3′ UTR of SEQ ID NO: 35.

24 hours following transfection, cells were lysed in RIPA buffer for subsequent western blotting. 10 μg of total protein from each sample was loaded into SDS-PAGE gels and electrophoresis was performed. Separated proteins were then transferred to PVDF membranes using the iBlot 2 Blotting System (Thermo-Fisher Scientific) using the vendor's recommended conditions. The membrane was then probed with anti-HFE antibody (Abcam) and anti-cyclophilin B (as an endogenous control) antibody. Secondary antibody detection was performed by ECL substrate and the blot was imaged on a commercially available imager.

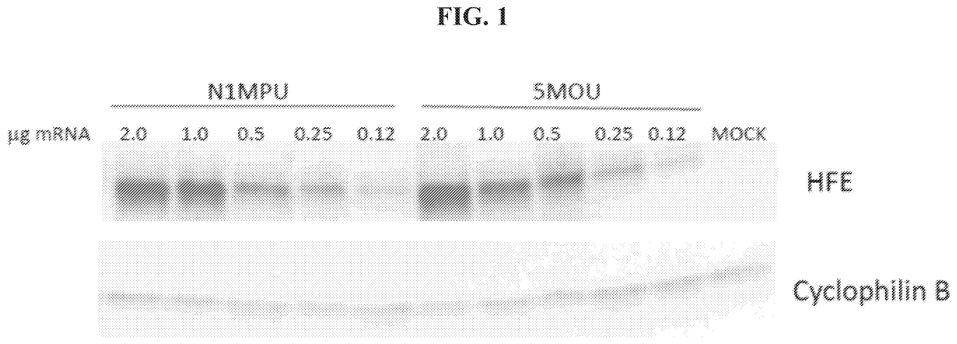

Results are illustrated in FIG. 1, and demonstrate that significant and concentration-dependent exogenous HFE protein expression is achieved relative to MOCK-transfected cells, as shown by increased signal intensity at higher amounts of transfected mRNA.

The data suggests that significant amounts of exogenous HFE protein can be translated by codon-optimized mRNAs (containing either N1-methyl-pseudouridine or 5-methoxyuridine).

### Example 2: Duration of HFE Protein Expression in Hepatocytes from HFE mRNA

This example demonstrates the duration of exogenous HFE expression following transfection of HFE mRNA using a commercially available delivery agent.

Human primary hepatocytes were purchased (Thermo-Fisher Scientific), cryo-recovered, and plated according to the vendor's recommended procedure. 500 ng of codon-optimized HFE-coding mRNA (modified to replace uridines with either N1-methyl-pseudouridine [“N1”] or 5-methoxyuridine [“5MU”]) was transfected using Lipofectamine MessengerMAX (Thermo-Fisher Scientific). The RNA sequence used in this example is illustrated in SEQ ID NO: 67, which comprises a 5′-cap (providing a single 5′ A residue), a 5′ UTR of SEQ ID NO: 33, an HFE coding sequence of SEQ ID NO: 4 (encoding the protein of SEQ ID NO: 32), and a 3′ UTR of SEQ ID NO: 35.

24 hours following transfection (and every day subsequently), cells were lysed in RIPA buffer for western blotting. 10 μg of total protein from each sample timepoint was loaded into SDS-PAGE gels and electrophoresis was performed. Separated proteins were then transferred to PVDF membranes using the iBlot 2 Blotting System (Thermo-Fisher Scientific) using the vendor's recommended conditions. The membrane was then probed with anti-HFE antibody (Abcam) and anti-cyclophilin B (as an endogenous control) antibody. Direct detection was performed using secondary antibodies labeled with near-infrared (NIR) fluorescent dyes and the blot was imaged on a commercially available imager.

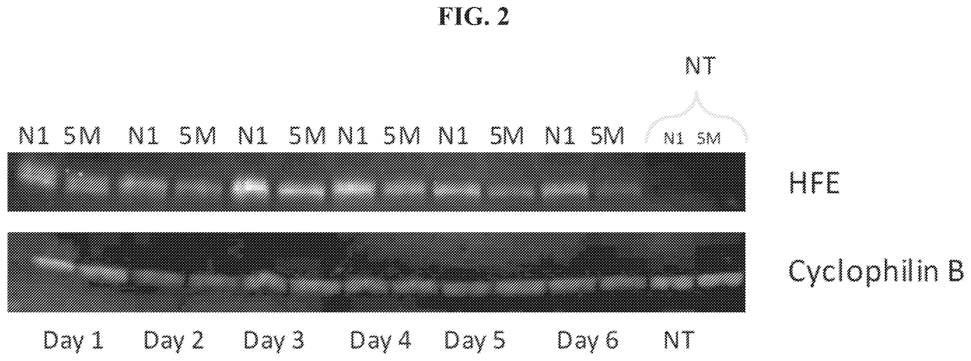

Results are illustrated in FIG. 2, and demonstrate that significant amounts of exogenous HFE protein expression is detected for up to 6 days following transfection relative to MOCK-transfected cells.

The data suggests that durable HFE expression can be obtained with a single administration of mRNA in human hepatocytes.

### Example 3: Expression of Liver HFE Protein and Reduction of Peripheral Iron Levels in Hfe Knockout Mice

This example demonstrates that administering an mRNA encoding HFE that is encapsulated in a lipid nanoparticle can produce liver HFE protein expression in Hfe knockout mice in a dose-dependent manner.

In this example, mRNA capable of encoding for human HFE (SEQ ID NO: 67) was encapsulated in a lipid nanoparticle and administered to Hfe knockout mice at 0.3 mg/kg, 1 mg/kg, and 3 mg/kg via tail vein injection. Approximately 48 hours after dosing, livers were harvested and blood collected to be assayed for iron levels.

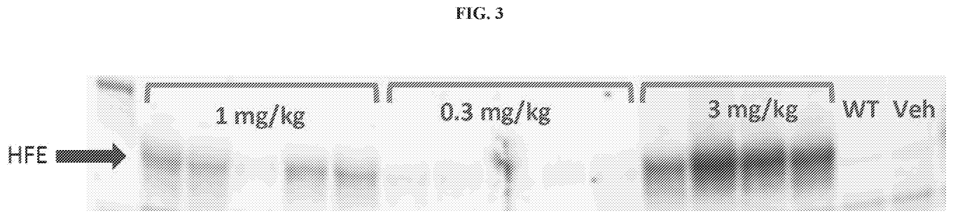

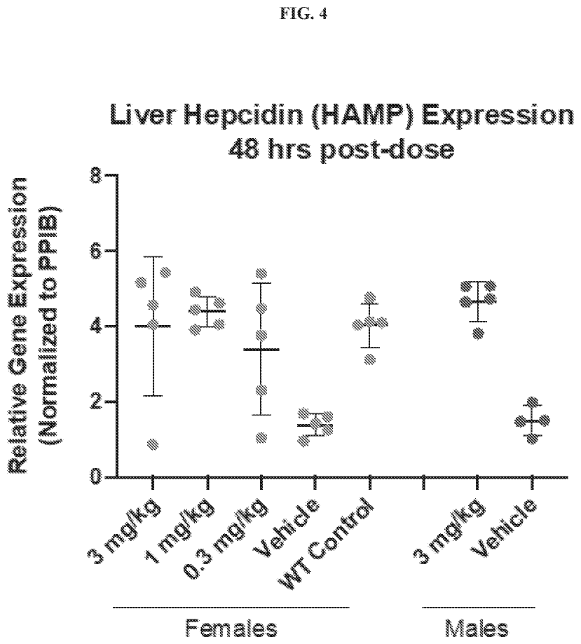

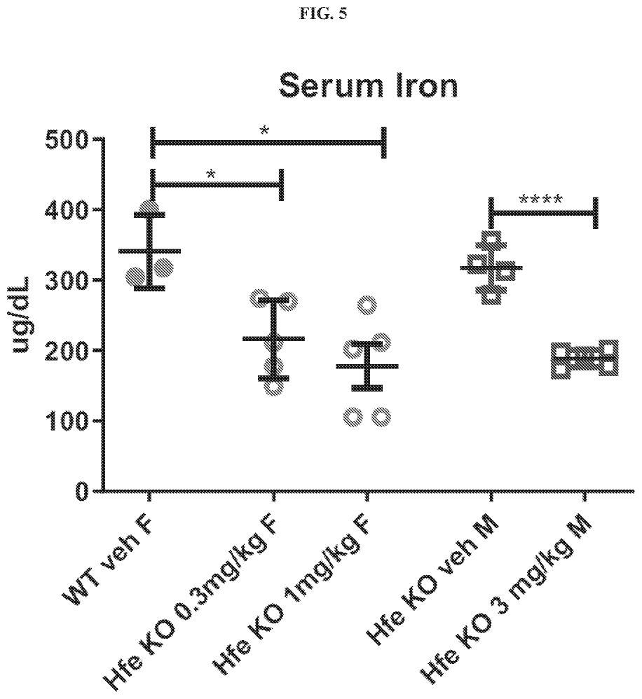

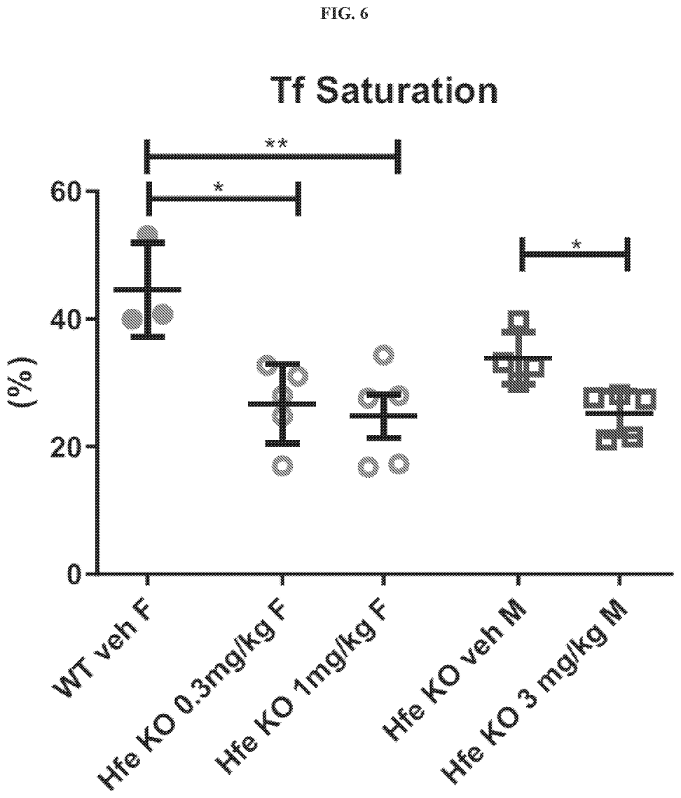

Following the single dose of HFE-encoding mRNA, dose-dependent HFE protein expression was observed in mouse liver homogenates by anti-HFE immunoblot (FIG. 3). Subsequent restoration of hepcidin gene expression was observed in treated mice via a branched DNA (bDNA) assay, with levels similar to wild-type controls (FIG. 4). In conjunction with the increases in HFE and hepcidin expression, reductions of serum iron (FIG. 5) and transferrin saturation (FIG. 6) levels were observed in the blood following a single dose of HFE-encoding mRNA-LNP.

These findings suggest that the combination of an HFE encoding mRNA and a lipid nanoparticle delivery system has promise for the treatment of hemochromatosis.

### Example 4: Reduction in Liver Iron in Hfe Knockout Mice Treated with HFE-Encoding mRNA

This example demonstrates that administration of a lipid nanoparticle encapsulated mRNA encoding HFE can reduce liver iron levels in Hfe knockout mice.

In this example, mRNA capable of encoding for human HFE (SEQ ID NO: 67) was encapsulated in a lipid nanoparticle and administered to Hfe knockout mice at 1 mg/kg via tail vein injection. At 7 days post-dosing, livers were harvested for subsequent analysis.

To measure liver iron concentrations, livers were first weighed dry and digested in 3 M HCl, 10% TCA mixture at 65° C. overnight. Next, digested extracts were mixed with bathophenalthroline chromagen reagent prior to measurement of 535 nm absorbance on a spectrophotometer. Absorbance values were quantified against a standard curve of known Fe concentrations.

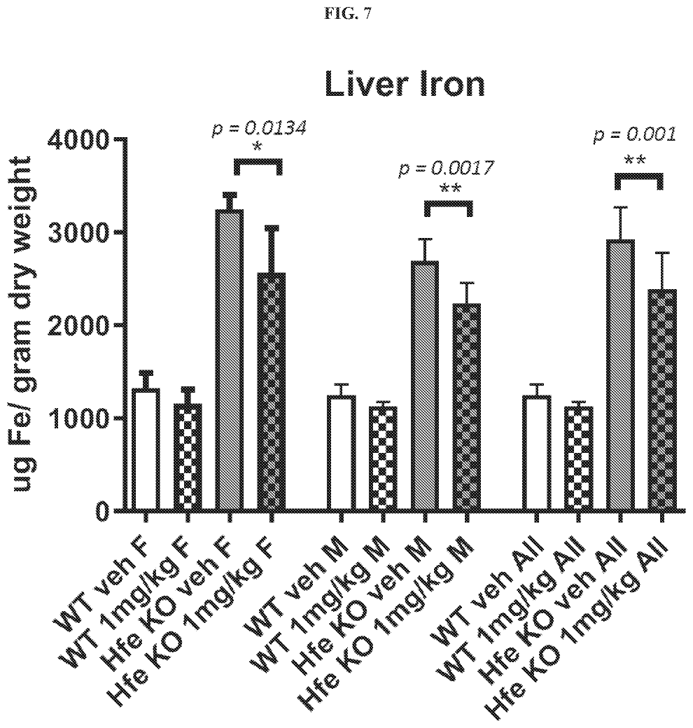

As shown in FIG. 7, reductions of liver iron levels (˜20%) were observed in Hfe knockout mice following a single intravenous dose of 1 mg/kg HFE-encoding mRNA. The effect was observed in both male and female cohorts of HH mice.

All publications, patents and literature specifically mentioned herein are incorporated by reference in their entireties for all purposes.

What is claimed is:

1. A polynucleotide for expressing a human hereditary hemochromatosis protein (HFE), or a fragment thereof, wherein the polynucleotide comprises natural and modified nucleotides and is expressible to provide the human HFE or a fragment thereof having HFE activity.

2. The polynucleotide of claim 1, wherein the polynucleotide is codon-optimized as compared to human HFE wild type mRNA.

3. The polynucleotide of claim 1, wherein the modified nucleotides are selected from
5-hydroxycytidine, 5-methylcytidine, 5-hydroxymethylcytidine, 5-carboxycytidine, 5-formylcytidine, 5-methoxycytidine, 5-propynylcytidine, 2-thiocytidine;
5-hydroxyuridine, 5-methyluridine, 5,6-dihydro-5-methyluridine, 2′-O-methyluridine, 2′-O-methyl-5-methyluridine, 2′-fluoro-2′-deoxyuridine, 2′-amino-2′-deoxyuridine, 2′-azido-2′-deoxyuridine, 4-thiouridine, 5-hydroxymethyluridine, 5-carboxyuridine, 5-carboxymethylesteruridine, 5-formyluridine, 5-methoxyuridine, 5-propynyluridine, 5-bromouridine, 5-iodouridine, 5-fluorouridine;
pseudouridine, 2′-O-methyl-pseudouridine, N¹-hydroxypseudouridine, N¹-methylpseudouridine, 2′-O-methyl-N¹-methylpseudouridine, N¹-ethylpseudouridine, N¹-hydroxymethylpseudouridine, and arauridine;
N⁶-methyladenosine, 2-aminoadenosine, 3-methyladenosine, 7-deazaadenosine, 8-oxoadenosine, inosine;
thienoguanosine, 7-deazaguanosine, 8-oxoguanosine, and 6-O-methylguanine.

4. The polynucleotide of claim 1, wherein the modified nucleotides are 5-methoxyuridines.

5. The polynucleotide of claim 1, wherein the modified nucleotides are N¹-methylpseudouridines.

6. The polynucleotide of claim 1, wherein the modified nucleotides are a combination of pseudouridines and N¹-methylpseudouridines.

7. The polynucleotide of claim 1, wherein the modified nucleotides are a combination of 5-methoxyuridines and N¹-methylpseudouridines.

8. The polynucleotide of claim 1, wherein the polynucleotide comprises a 5′-cap, a 5′ untranslated region, a coding region, a 3′ untranslated region, and a tail region.

9. The polynucleotide of claim 1, wherein the polynucleotide is translatable in a mammalian cell to express the human HFE or a fragment thereof having HFE activity.

10. The polynucleotide of claim 1, wherein the polynucleotide is translatable in a subject in vivo to express the human HFE or a fragment thereof having HFE activity.

11. The polynucleotide of claim 1, wherein the polynucleotide has reduced immunogenicity as compared to a human HFE wild-type mRNA.

12. The polynucleotide of claim 1, wherein the polynucleotide comprises a nucleobase sequence selected from SEQ ID NOs: 4-31.

13. The polynucleotide of claim 8, wherein the 5′-cap comprises N7-Methyl-Gppp(2′-O-Methyl-A).

14. The polynucleotide of claim 8, wherein the 5′ untranslated region comprises or consists of SEQ ID NO: 33.

15. The polynucleotide of claim 8, wherein the 3′ untranslated region comprises or consists of SEQ ID NO: 35.

16. The polynucleotide of claim 8, wherein the tail region is a polyA tail region.

17. The polynucleotide of claim 16, wherein the polyA tail region is 60 to 220 adenosine nucleotides.

18. The polynucleotide of claim 17, wherein the polyA tail region is about 80 nucleotides in length.

19. A polynucleotide comprising a nucleobase sequence that is at least 95% identical to a nucleobase sequence selected from SEQ ID NOs: 4-31.

20. The polynucleotide of claim 19, wherein the polynucleotide comprises a nucleobase sequence that is at least 99% identical to a nucleobase sequence selected from SEQ ID NOs: 4-31.

21. The polynucleotide of claim 19, wherein the polynucleotide comprises a nucleobase selected from SEQ ID NOs: 4-31.

22. The polynucleotide of claim 19, wherein at least one uridine nucleotide is replaced with a 5-methoxyuridine nucleotide.

23. The polynucleotide of claim 19, wherein all uridine nucleotides are replaced with 5-methoxyuridine nucleotide.

24. The polynucleotide of claim 19, wherein at least one uridine nucleotide is replaced with a N¹-methylpseudouridine nucleotide.

25. The polynucleotide of claim 19, wherein all uridine nucleotides are replaced with 5-methoxyuridine nucleotide.

26. The polynucleotide of claim 19, wherein the polynucleotide further comprises a 5′-cap, a 5′ untranslated region, a 3′ untranslated region, and/or a tail region.

27. The polynucleotide of claim 26, wherein the 5′-cap comprises N7-Methyl-Gppp(2′-O-Methyl-A).

28. The polynucleotide of claim 26, wherein the 5′ untranslated region comprises or consists of SEQ ID NO: 33.

29. The polynucleotide of claim 26, wherein the 3′ untranslated region comprises or consists of SEQ ID NO: 35.

30. The polynucleotide of claim 26, wherein the tail region is a polyA tail region.

31. The polynucleotide of claim 30, wherein the polyA tail region is 60 to 220 adenosine nucleotides.

32. The polynucleotide of claim 31, wherein the polyA tail region is about 80 nucleotides in length.

33. The polynucleotide of claim 19, wherein the polynucleotide comprises the nucleobase sequence of SEQ ID NO: 4.

34. A composition comprising one or more polynucleotides of any of claims 1-33, and a pharmaceutically acceptable carrier.

35. The composition of claim 34, wherein the carrier comprises a transfection reagent, a lipid nanoparticle, or a liposome.

36. The composition of claim 35, wherein the carrier is a lipid nanoparticle.

37. The composition of claim 36, wherein the lipid nanoparticle comprises a cationic lipid selected from ATX-002, ATX-081, ATX-095, or ATX-126.

38. A composition of any of claims 34-37 for use in medical therapy.

39. A composition of any of claims 34-37 for use in the treatment of a human or animal body.

40. The use of a composition of any of claims 34-37 for preparing or manufacturing a medicament for ameliorating, preventing, delaying onset, or treating a disease or disorder associated with reduced activity of hereditary hemochromatosis protein (HFE) in a subject need thereof.

41. The use of claim 40, wherein the disease is hereditary hemochromatosis.

42. A method for ameliorating, preventing, delaying onset, or treating a disease or disorder associated with reduced activity of hereditary hemochromatosis protein (HFE) in a subject need thereof, the method comprising administering to the subject a composition of any of claims 34-37.

43. The method of claim 42, wherein the disease is hereditary hemochromatosis.

44. A method for ameliorating, preventing, delaying onset, or treating hemochromatosis in a subject need thereof, the method comprising administering to the subject a composition of any of claims 34-37.

45. The method of claim 44, wherein the hemochromatosis is selected from hereditary hemochromatosis and secondary hemochromatosis.

46. The method of claim 45, wherein the hemochromatosis is hereditary hemochromatosis.

47. The method of claim 45, wherein the hemochromatosis is secondary hemochromatosis.

48. The method of any of claims 42-47, wherein the administration is intravenous, subcutaneous, pulmonary, intramuscular, intraperitoneal, dermal, oral, nasal, or inhalation.

49. The method of any of claims 42-48, wherein the administration is once daily, weekly, every two weeks, monthly, every two months, quarterly, or yearly.

50. The method of any of claims 42-49, wherein the administration comprises an effective dose of from 0.01 to 10 mg/kg.

51. The method of any of claims 42-49, wherein the composition is administered at a dose of about 0.1, 0.3, 0.5, 1, 3, 5, or about 10 mg/kg.

52. The method of any of claims 42-51, wherein the administration increases expression of HFE in the liver of the subject.

53. A kit for expressing a human HFE in vivo, the kit comprising a 0.1 to 500 mg dose of one or more polynucleotides of any of claims 1-33 and a device for administering the dose.

54. The kit of claim 53, wherein the device is an injection needle, an intravenous needle, or an inhalation device.

55. A polynucleotide comprising a nucleobase sequence that is less than 95% identical to the wild-type human HFE coding sequence over the full length human HFE coding sequence of SEQ ID NO: 1, and wherein the human HFE coding sequence is at least 95% identical to a sequence selected from SEQ ID NOs: 4-31.

56. A polynucleotide consisting of a nucleobase sequence that is less than 95% identical to the wild-type human HFE coding sequence over the full length human HFE coding sequence of SEQ ID NO: 1, and wherein the human HFE coding sequence is at least 95% identical to a sequence selected from SEQ ID NOs: 4-31.

57. A polynucleotide comprising a nucleobase sequence that is less than 95% identical to the wild-type human HFE coding sequence over the full length human HFE coding sequence of SEQ ID NO: 1, and wherein the human HFE coding sequence is at least 95% identical to SEQ ID NO: 4.

58. A polynucleotide comprising a nucleobase sequence that is at least 98% identical to a sequence selected from SEQ ID NOs: 4-31.

59. A polynucleotide comprising a nucleobase sequence that is at least 99% identical to a sequence selected from SEQ ID NOs: 4-31.

60. A polynucleotide comprising a nucleobase sequence selected from SEQ ID NOs: 4-31.

61. A polynucleotide comprising a nucleobase sequence of SEQ ID NO: 4.

62. A polynucleotide comprising a nucleobase sequence of SEQ ID NO: 67.


SEQUENCE LISTING (67 sequences)

SEQ ID NO: 1 - RNA, 1044 nt, Homo sapiens
augggcccgcgagccaggccggcgcuucuccuccugaugcuuuugcagaccgcgguccugcaggggcgcuugcugcguucacacucucugcacuaccucuucaugggugccucagagcaggaccuuggucuuuccuuguuugaagcuuugggcuacguggaugaccagcuguucguguucuaugaucaugagagucgccguguggagccccgaacuccauggguuuccaguagaauuucaagccagauguggcugcagcugagucagagucugaaagggugggaucacauguucacuguugacuucuggacuauuauggaaaaucacaaccacagcaaggagucccacacccugcaggucauccugggcugugaaaugcaagaagacaacaguaccgagggcuacuggaaguacggguaugaugggcaggaccaccuugaauucugcccugacacacuggauuggagagcagcagaacccagggccuggcccaccaagcuggagugggaaaggcacaagauucgggccaggcagaacagggccuaccuggagagggacugcccugcacagcugcagcaguugcuggagcuggggagagguguuuuggaccaacaagugccuccuuuggugaaggugacacaucaugugaccucuucagugaccacucuacggugucgggccuugaacuacuacccccagaacaucaccaugaaguggcugaaggauaagcagccaauggaugccaaggaguucgaaccuaaagacguauugcccaauggggaugggaccuaccagggcuggauaaccuuggcuguacccccuggggaagagcagagauauacgugccagguggagcacccaggccuggaucagccccucauugugaucugggagcccucaccgucuggcacccuagucauuggagucaucaguggaauugcuguuuuugucgucaucuuguucauuggaauuuuguucauaauauuaaggaagaggcaggguucaagaggagccauggggcacuacgucuuagcugaacgugag

SEQ ID NO: 2 - protein, 345 aa, Homo sapiens
MGPRARPALLLLMLLQTAVLQGRLLRSHSLHYLFMGASEQDLGLSLFEALGYVDDQLFVFYDHESRRVEPRTPWVSSRISSQMWLQLSQSLKGWDHMFTVDFWTIMENHNHSKESHTLQVILGCEMQEDNSTEGYWKYGYDGQDHLEFCPDTLDWRAAEPRAWPTKLEWERHKIRARQNRAYLERDCPAQLQQLLELGRGVLDQQEGSESSSLPGKVTTLRCRALNYYPQNITMKWLKDKQPMDAKEFEPKDVLPNGDGTYQGWITLAVPPGEEQRYTCQVEHPGLDQPLIVIWEPSPSGTLVIGVISGIAVFVVILFIGILFIILRKRQGSRGAMGHYVLAERE

SEQ ID NO: 3 - protein, 337 aa, Homo sapiens
MGPRARPALLLLMLLQTAVLQGRLLRSHSLHYLFMGASEQDLGLSLFEALGYVDDQLFVFYDHESRRVEPRTPWVSSRISSQMWLQLSQSLKGWDHMFTVDFWTIMENHNHSKESHTLQVILGCEMQEDNSTEGYWKYGYDGQDHLEFCPDTLDWRAAEPRAWPTKLEWERHKIRARQNRAYLERDCPAQLQQLLELGRGVLDQQVPPLVKVTHHVTSSVTTLRCRALNYYPQNITMKWLKDKQPMDAKEFEPKDVLPNGDGTYQGWITLAVPPGEEQRYTCQVEHPGLDQPLIVIWEPSPSGTLVIGVISGIAVFVVILFIGILFIILRKRQGSSF

SEQ ID NO: 4 - RNA, 1044 nt, Artificial Sequence
  HFE Coding Sequence - Codon-Optimized
augggcccccgggcccggcccgcccugcugcugcugaugcugcugcagaccgccgugcugcagggccggcugcugcggagccacagccugcacuaccuguucaugggcgccagcgagcaggaccugggccugagccuguucgaggcccugggcuacguggacgaccagcuguucguguucuacgaccacgagagccggcggguggagccccggacccccugggugagcagccggaucagcagccagauguggcugcagcugagccagagccugaagggcugggaccacauguucaccguggacuucuggaccaucauggagaaccacaaccacagcaaggagagccacacccugcaggugauccugggcugcgagaugcaggaggacaacagcaccgagggcuacuggaaguacggcuacgacggccaggaccaccuggaguucugccccgacacccuggacuggcgggccgccgagccccgggccuggcccaccaagcuggagugggagcggcacaagauccgggcccggcagaaccgggccuaccuggagcgggacugccccgcccagcugcagcagcugcuggagcugggccggggcgugcuggaccagcaggugcccccccuggugaaggugacccaccacgugaccagcagcgugaccacccugcggugccgggcccugaacuacuacccccagaacaucaccaugaaguggcugaaggacaagcagcccauggacgccaaggaguucgagcccaaggacgugcugcccaacggcgacggcaccuaccagggcuggaucacccuggccgugccccccggcgaggagcagcgguacaccugccagguggagcaccccggccuggaccagccccugaucgugaucugggagcccagccccagcggcacccuggugaucggcgugaucagcggcaucgccguguucguggugauccuguucaucggcauccuguucaucauccugcggaagcggcagggcagccggggcgccaugggccacuacgugcuggccgagcgggag

SEQ ID NO: 5 - RNA, 1044 nt, Artificial Sequence
  HFE Coding Sequence - Codon-Optimized
augggcccccgggccaggcccgcccugcugcugcugaugcuguugcagaccgccgugcugcagggccgguugcugcggucccacucccugcacuaccuguucaugggcgccuccgagcaggaccugggccuguccuuguucgaggccuugggcuacguggacgaccagcuguucguguucuacgaccacgagagccggcggguggagccccggacccccuggguguccagcaggaucuccagccagauguggcugcagcugagccagagccugaagggcugggaccacauguucaccguggacuucuggaccaucauggagaaccacaaccacagcaaggagucccacacccugcaggugauccugggcugcgagaugcaggaggacaacagcaccgagggcuacuggaaguacggcuacgacggccaggaccaccuggaguucugccccgacacccuggacuggagggccgccgagcccagggccuggcccaccaagcuggagugggagaggcacaagauccgggccaggcagaacagggccuaccuggagagggacugccccgcccagcugcagcaguugcuggagcugggcaggggcguguuggaccagcaggugccccccuuggugaaggugacccaccacgugaccuccuccgugaccacccugcggugccgggccuugaacuacuacccccagaacaucaccaugaaguggcugaaggacaagcagcccauggacgccaaggaguucgagcccaaggacguguugcccaacggcgacggcaccuaccagggcuggaucaccuuggccgugccccccggcgaggagcagagguacaccugccagguggagcaccccggccuggaccagccccugaucgugaucugggagcccucccccuccggcacccuggugaucggcgugaucagcggcaucgccguguucguggugaucuuguucaucggcaucuuguucaucaucuugaggaagaggcagggcuccaggggcgccaugggccacuacguguuggccgagcgggag

SEQ ID NO: 6 - RNA, 1044 nt, Artificial Sequence
  HFE Coding Sequence - Codon-Optimized
auggggccgagggcgaggccggcgcuccuccuccucaugcuccuccaaacggcggugcuccaagggaggcuccucaggagccacagccuccacuaccucuucaugggggcgagcgaacaagaccucgggcucagccucuucgaagcgcucggguacguggacgaccaacucuucguguucuacgaccacgaaagcaggaggguggaaccgaggacgccgugggugagcagcaggaucagcagccaaauguggcuccaacucagccaaagccucaaagggugggaccacauguucacgguggacuucuggacgaucauggaaaaccacaaccacagcaaagaaagccacacgcuccaagugauccucgggugcgaaaugcaagaagacaacagcacggaaggguacuggaaauacggguacgacgggcaagaccaccucgaauucugcccggacacgcucgacuggagggcggcggaaccgagggcguggccgacgaaacucgaaugggaaaggcacaaaaucagggcgaggcaaaacagggcguaccucgaaagggacugcccggcgcaacuccaacaacuccucgaacucgggaggggggugcucgaccaacaagugccgccgcucgugaaagugacgcaccacgugacgagcagcgugacgacgcucaggugcagggcgcucaacuacuacccgcaaaacaucacgaugaaauggcucaaagacaaacaaccgauggacgcgaaagaauucgaaccgaaagacgugcucccgaacggggacgggacguaccaaggguggaucacgcucgcggugccgccgggggaagaacaaagguacacgugccaaguggaacacccggggcucgaccaaccgcucaucgugaucugggaaccgagcccgagcgggacgcucgugaucggggugaucagcgggaucgcgguguucguggugauccucuucaucgggauccucuucaucauccucaggaaaaggcaagggagcaggggggcgauggggcacuacgugcucgcggaaagggaa

SEQ ID NO: 7 - RNA, 1044 nt, Artificial Sequence
  HFE Coding Sequence - Codon-Optimized
auggggccaagggcaaggccagcacuacuacuacuaaugcuacuacaaacagcaguacuacaagguaggcuacuaaggucacauucacuacauuaucuauuuaugggugcaucagaacaagaucuaggucuaucacuauuugaagcacuaggguauguggaugaucagcuguuuguguuuuaugaucaugagucuaggaggguggagccuaggacaccuugggugucuucuaggauuucuucucagauguggcugcagcugucucagucucugaaggggugggaucauauguuuacaguggauuuuuggacaauuauggagaaucauaaucauucuaaggagucucauacacugcaggugauucuggggugugagaugcaggaggauaauucuacagagggguauuggaaguauggguaugaugggcaggaucaucuggaguuuuguccugauacacuggauuggagggcugcugagccuagggcuuggccuacaaagcuggagugggagaggcauaagauuagggcuaggcagaauagggcuuaucuggagagggauuguccugcucagcugcagcagcugcuggagcuggggaggggggugcuggaucagcaggugccuccucuggugaaggugacacaucaugugacaucuucugugacaacacugagguguagggcucugaauuauuauccucagaauauuacaaugaaguggcugaaggauaagcagccuauggaugcuaaggaguuugagccuaaggaugugcugccuaauggggaugggacauaucagggguggauuacacuggcugugccuccuggggaggagcagagguauacaugucagguggagcauccugggcuggaucagccucugauugugauuugggagccuucuccuucugggacacuggugauuggggugauuucugggauugcuguguuuguggugauucuguuuauugggauucuguuuauuauucugaggaagaggcaggggucuaggggggcuauggggcauuaugugcuggcugagagggag

SEQ ID NO: 8 - RNA, 1044 nt, Artificial Sequence
  HFE Coding Sequence - Codon-Optimized
augggcccccgggcucggcccgcucugcugcugcugaugcugcugcagaccgcugugcugcagggccggcugcugcggagccacagccugcacuaccuguucaugggcgcuagcgagcaggaccugggccugagccuguucgaggcucugggcuacguggacgaccagcuguucguguucuacgaccacgagagccggcggguggagccccggacccccugggugagcagccggaucagcagccagauguggcugcagcugagccagagccugaagggcugggaccacauguucaccguggacuucuggaccaucauggagaaccacaaccacagcaaggagagccacacccugcaggugauccugggcugcgagaugcaggaggacaacagcaccgagggcuacuggaaguacggcuacgacggccaggaccaccuggaguucugccccgacacccuggacuggcgggcugcugagccccgggcuuggcccaccaagcuggagugggagcggcacaagauccgggcucggcagaaccgggcuuaccuggagcgggacugccccgcucagcugcagcagcugcuggagcugggccggggcgugcuggaccagcaggugcccccccuggugaaggugacccaccacgugaccagcagcgugaccacccugcggugccgggcucugaacuacuacccccagaacaucaccaugaaguggcugaaggacaagcagcccauggacgcuaaggaguucgagcccaaggacgugcugcccaacggcgacggcaccuaccagggcuggaucacccuggcugugccccccggcgaggagcagcgguacaccugccagguggagcaccccggccuggaccagccccugaucgugaucugggagcccagccccagcggcacccuggugaucggcgugaucagcggcaucgcuguguucguggugauccuguucaucggcauccuguucaucauccugcggaagcggcagggcagccggggcgcuaugggccacuacgugcuggcugagcgggag

SEQ ID NO: 9 - RNA, 1044 nt, Artificial Sequence
  HFE Coding Sequence - Codon-Optimized
augggcccccgggcccggcccgcccugcugcugcugaugcugcugcagaccgccgugcugcagggccggcugcugcggagccacagccugcacuaccuguucaugggcgccagcgagcaggaccugggccugagccuguucgaggcccugggcuacguggacgaccagcuguucguguucuacgaccacgagagccggcggguggagccccggacccccugggugagcagccggaucagcagccagauguggcugcagcugagccagagccugaagggcugggaccacauguucaccguggacuucuggaccaucauggagaaccacaaccacagcaaggagagccacacccugcaggugauccugggcugugagaugcaggaggacaacagcaccgagggcuacuggaaguacggcuacgacggccaggaccaccuggaguucugucccgacacccuggacuggcgggccgccgagccccgggccuggcccaccaagcuggagugggagcggcacaagauccgggcccggcagaaccgggccuaccuggagcgggacugucccgcccagcugcagcagcugcuggagcugggccggggcgugcuggaccagcaggugcccccccuggugaaggugacccaccacgugaccagcagcgugaccacccugcggugucgggcccugaacuacuacccccagaacaucaccaugaaguggcugaaggacaagcagcccauggacgccaaggaguucgagcccaaggacgugcugcccaacggcgacggcaccuaccagggcuggaucacccuggccgugccccccggcgaggagcagcgguacaccugucagguggagcaccccggccuggaccagccccugaucgugaucugggagcccagccccagcggcacccuggugaucggcgugaucagcggcaucgccguguucguggugauccuguucaucggcauccuguucaucauccugcggaagcggcagggcagccggggcgccaugggccacuacgugcuggccgagcgggag

SEQ ID NO: 10 - RNA, 1044 nt, Artificial Sequence
  HFE Coding Sequence - Codon-Optimized
augggcccccgggcccggcccgcccugcugcugcugaugcugcugcagaccgccgugcugcagggccggcugcugcggagccacagccugcacuaccuguucaugggcgccagcgagcaggaucugggccugagccuguucgaggcccugggcuacguggaugaucagcuguucguguucuacgaucacgagagccggcggguggagccccggacccccugggugagcagccggaucagcagccagauguggcugcagcugagccagagccugaagggcugggaucacauguucaccguggauuucuggaccaucauggagaaccacaaccacagcaaggagagccacacccugcaggugauccugggcugcgagaugcaggaggauaacagcaccgagggcuacuggaaguacggcuacgauggccaggaucaccuggaguucugccccgauacccuggauuggcgggccgccgagccccgggccuggcccaccaagcuggagugggagcggcacaagauccgggcccggcagaaccgggccuaccuggagcgggauugccccgcccagcugcagcagcugcuggagcugggccggggcgugcuggaucagcaggugcccccccuggugaaggugacccaccacgugaccagcagcgugaccacccugcggugccgggcccugaacuacuacccccagaacaucaccaugaaguggcugaaggauaagcagcccauggaugccaaggaguucgagcccaaggaugugcugcccaacggcgauggcaccuaccagggcuggaucacccuggccgugccccccggcgaggagcagcgguacaccugccagguggagcaccccggccuggaucagccccugaucgugaucugggagcccagccccagcggcacccuggugaucggcgugaucagcggcaucgccguguucguggugauccuguucaucggcauccuguucaucauccugcggaagcggcagggcagccggggcgccaugggccacuacgugcuggccgagcgggag

SEQ ID NO: 11 - RNA, 1044 nt, Artificial Sequence
  HFE Coding Sequence - Codon-Optimized
augggcccccgggcccggcccgcccugcugcugcugaugcugcugcagaccgccgugcugcagggccggcugcugcggagccacagccugcacuaccuguucaugggcgccagcgaacaggaccugggccugagccuguucgaagcccugggcuacguggacgaccagcuguucguguucuacgaccacgaaagccggcggguggaaccccggacccccugggugagcagccggaucagcagccagauguggcugcagcugagccagagccugaagggcugggaccacauguucaccguggacuucuggaccaucauggaaaaccacaaccacagcaaggaaagccacacccugcaggugauccugggcugcgaaaugcaggaagacaacagcaccgaaggcuacuggaaguacggcuacgacggccaggaccaccuggaauucugccccgacacccuggacuggcgggccgccgaaccccgggccuggcccaccaagcuggaaugggaacggcacaagauccgggcccggcagaaccgggccuaccuggaacgggacugccccgcccagcugcagcagcugcuggaacugggccggggcgugcuggaccagcaggugcccccccuggugaaggugacccaccacgugaccagcagcgugaccacccugcggugccgggcccugaacuacuacccccagaacaucaccaugaaguggcugaaggacaagcagcccauggacgccaaggaauucgaacccaaggacgugcugcccaacggcgacggcaccuaccagggcuggaucacccuggccgugccccccggcgaagaacagcgguacaccugccagguggaacaccccggccuggaccagccccugaucgugaucugggaacccagccccagcggcacccuggugaucggcgugaucagcggcaucgccguguucguggugauccuguucaucggcauccuguucaucauccugcggaagcggcagggcagccggggcgccaugggccacuacgugcuggccgaacgggaa

SEQ ID NO: 12 - RNA, 1044 nt, Artificial Sequence
  HFE Coding Sequence - Codon-Optimized
augggcccccgggcccggcccgcccugcugcugcugaugcugcugcagaccgccgugcugcagggccggcugcugcggagccacagccugcacuaccuguuuaugggcgccagcgagcaggaccugggccugagccuguuugaggcccugggcuacguggacgaccagcuguuuguguuuuacgaccacgagagccggcggguggagccccggacccccugggugagcagccggaucagcagccagauguggcugcagcugagccagagccugaagggcugggaccacauguuuaccguggacuuuuggaccaucauggagaaccacaaccacagcaaggagagccacacccugcaggugauccugggcugcgagaugcaggaggacaacagcaccgagggcuacuggaaguacggcuacgacggccaggaccaccuggaguuuugccccgacacccuggacuggcgggccgccgagccccgggccuggcccaccaagcuggagugggagcggcacaagauccgggcccggcagaaccgggccuaccuggagcgggacugccccgcccagcugcagcagcugcuggagcugggccggggcgugcuggaccagcaggugcccccccuggugaaggugacccaccacgugaccagcagcgugaccacccugcggugccgggcccugaacuacuacccccagaacaucaccaugaaguggcugaaggacaagcagcccauggacgccaaggaguuugagcccaaggacgugcugcccaacggcgacggcaccuaccagggcuggaucacccuggccgugccccccggcgaggagcagcgguacaccugccagguggagcaccccggccuggaccagccccugaucgugaucugggagcccagccccagcggcacccuggugaucggcgugaucagcggcaucgccguguuuguggugauccuguuuaucggcauccuguuuaucauccugcggaagcggcagggcagccggggcgccaugggccacuacgugcuggccgagcgggag

SEQ ID NO: 13 - RNA, 1044 nt, Artificial Sequence
  HFE Coding Sequence - Codon-Optimized
auggggccccgggcccggcccgcccugcugcugcugaugcugcugcagaccgccgugcugcaggggcggcugcugcggagccacagccugcacuaccuguucaugggggccagcgagcaggaccuggggcugagccuguucgaggcccugggguacguggacgaccagcuguucguguucuacgaccacgagagccggcggguggagccccggacccccugggugagcagccggaucagcagccagauguggcugcagcugagccagagccugaaggggugggaccacauguucaccguggacuucuggaccaucauggagaaccacaaccacagcaaggagagccacacccugcaggugauccuggggugcgagaugcaggaggacaacagcaccgagggguacuggaaguacggguacgacgggcaggaccaccuggaguucugccccgacacccuggacuggcgggccgccgagccccgggccuggcccaccaagcuggagugggagcggcacaagauccgggcccggcagaaccgggccuaccuggagcgggacugccccgcccagcugcagcagcugcuggagcuggggcggggggugcuggaccagcaggugcccccccuggugaaggugacccaccacgugaccagcagcgugaccacccugcggugccgggcccugaacuacuacccccagaacaucaccaugaaguggcugaaggacaagcagcccauggacgccaaggaguucgagcccaaggacgugcugcccaacggggacgggaccuaccagggguggaucacccuggccgugccccccggggaggagcagcgguacaccugccagguggagcaccccgggcuggaccagccccugaucgugaucugggagcccagccccagcgggacccuggugaucggggugaucagcgggaucgccguguucguggugauccuguucaucgggauccuguucaucauccugcggaagcggcaggggagccggggggccauggggcacuacgugcuggccgagcgggag

SEQ ID NO: 14 - RNA, 1044 nt, Artificial Sequence
  HFE Coding Sequence - Codon-Optimized
augggcccccgggcccggcccgcccugcugcugcugaugcugcugcagaccgccgugcugcagggccggcugcugcggagccauagccugcauuaccuguucaugggcgccagcgagcaggaccugggccugagccuguucgaggcccugggcuacguggacgaccagcuguucguguucuacgaccaugagagccggcggguggagccccggacccccugggugagcagccggaucagcagccagauguggcugcagcugagccagagccugaagggcugggaccauauguucaccguggacuucuggaccaucauggagaaccauaaccauagcaaggagagccauacccugcaggugauccugggcugcgagaugcaggaggacaacagcaccgagggcuacuggaaguacggcuacgacggccaggaccaucuggaguucugccccgacacccuggacuggcgggccgccgagccccgggccuggcccaccaagcuggagugggagcggcauaagauccgggcccggcagaaccgggccuaccuggagcgggacugccccgcccagcugcagcagcugcuggagcugggccggggcgugcuggaccagcaggugcccccccuggugaaggugacccaucaugugaccagcagcgugaccacccugcggugccgggcccugaacuacuacccccagaacaucaccaugaaguggcugaaggacaagcagcccauggacgccaaggaguucgagcccaaggacgugcugcccaacggcgacggcaccuaccagggcuggaucacccuggccgugccccccggcgaggagcagcgguacaccugccagguggagcaucccggccuggaccagccccugaucgugaucugggagcccagccccagcggcacccuggugaucggcgugaucagcggcaucgccguguucguggugauccuguucaucggcauccuguucaucauccugcggaagcggcagggcagccggggcgccaugggccauuacgugcuggccgagcgggag

SEQ ID NO: 15 - RNA, 1044 nt, Artificial Sequence
  HFE Coding Sequence - Codon-Optimized
augggcccccgggcccggcccgcccugcugcugcugaugcugcugcagaccgccgugcugcagggccggcugcugcggagccacagccugcacuaccuguucaugggcgccagcgagcaggaccugggccugagccuguucgaggcccugggcuacguggacgaccagcuguucguguucuacgaccacgagagccggcggguggagccccggacccccugggugagcagccggauuagcagccagauguggcugcagcugagccagagccugaagggcugggaccacauguucaccguggacuucuggaccauuauggagaaccacaaccacagcaaggagagccacacccugcaggugauucugggcugcgagaugcaggaggacaacagcaccgagggcuacuggaaguacggcuacgacggccaggaccaccuggaguucugccccgacacccuggacuggcgggccgccgagccccgggccuggcccaccaagcuggagugggagcggcacaagauucgggcccggcagaaccgggccuaccuggagcgggacugccccgcccagcugcagcagcugcuggagcugggccggggcgugcuggaccagcaggugcccccccuggugaaggugacccaccacgugaccagcagcgugaccacccugcggugccgggcccugaacuacuacccccagaacauuaccaugaaguggcugaaggacaagcagcccauggacgccaaggaguucgagcccaaggacgugcugcccaacggcgacggcaccuaccagggcuggauuacccuggccgugccccccggcgaggagcagcgguacaccugccagguggagcaccccggccuggaccagccccugauugugauuugggagcccagccccagcggcacccuggugauuggcgugauuagcggcauugccguguucguggugauucuguucauuggcauucuguucauuauucugcggaagcggcagggcagccggggcgccaugggccacuacgugcuggccgagcgggag

SEQ ID NO: 16 - RNA, 1044 nt, Artificial Sequence
  HFE Coding Sequence - Codon-Optimized
augggcccccgggcccggcccgcccugcugcugcugaugcugcugcagaccgccgugcugcagggccggcugcugcggagccacagccugcacuaccuguucaugggcgccagcgagcaggaccugggccugagccuguucgaggcccugggcuacguggacgaccagcuguucguguucuacgaccacgagagccggcggguggagccccggacccccugggugagcagccggaucagcagccagauguggcugcagcugagccagagccugaaaggcugggaccacauguucaccguggacuucuggaccaucauggagaaccacaaccacagcaaagagagccacacccugcaggugauccugggcugcgagaugcaggaggacaacagcaccgagggcuacuggaaauacggcuacgacggccaggaccaccuggaguucugccccgacacccuggacuggcgggccgccgagccccgggccuggcccaccaaacuggagugggagcggcacaaaauccgggcccggcagaaccgggccuaccuggagcgggacugccccgcccagcugcagcagcugcuggagcugggccggggcgugcuggaccagcaggugcccccccuggugaaagugacccaccacgugaccagcagcgugaccacccugcggugccgggcccugaacuacuacccccagaacaucaccaugaaauggcugaaagacaaacagcccauggacgccaaagaguucgagcccaaagacgugcugcccaacggcgacggcaccuaccagggcuggaucacccuggccgugccccccggcgaggagcagcgguacaccugccagguggagcaccccggccuggaccagccccugaucgugaucugggagcccagccccagcggcacccuggugaucggcgugaucagcggcaucgccguguucguggugauccuguucaucggcauccuguucaucauccugcggaaacggcagggcagccggggcgccaugggccacuacgugcuggccgagcgggag

SEQ ID NO: 17 - RNA, 1044 nt, Artificial Sequence
  HFE Coding Sequence - Codon-Optimized
augggcccccgggcccggcccgcccuccuccuccucaugcuccuccagaccgccgugcuccagggccggcuccuccggagccacagccuccacuaccucuucaugggcgccagcgagcaggaccucggccucagccucuucgaggcccucggcuacguggacgaccagcucuucguguucuacgaccacgagagccggcggguggagccccggacccccugggugagcagccggaucagcagccagauguggcuccagcucagccagagccucaagggcugggaccacauguucaccguggacuucuggaccaucauggagaaccacaaccacagcaaggagagccacacccuccaggugauccucggcugcgagaugcaggaggacaacagcaccgagggcuacuggaaguacggcuacgacggccaggaccaccucgaguucugccccgacacccucgacuggcgggccgccgagccccgggccuggcccaccaagcucgagugggagcggcacaagauccgggcccggcagaaccgggccuaccucgagcgggacugccccgcccagcuccagcagcuccucgagcucggccggggcgugcucgaccagcaggugcccccccucgugaaggugacccaccacgugaccagcagcgugaccacccuccggugccgggcccucaacuacuacccccagaacaucaccaugaaguggcucaaggacaagcagcccauggacgccaaggaguucgagcccaaggacgugcuccccaacggcgacggcaccuaccagggcuggaucacccucgccgugccccccggcgaggagcagcgguacaccugccagguggagcaccccggccucgaccagccccucaucgugaucugggagcccagccccagcggcacccucgugaucggcgugaucagcggcaucgccguguucguggugauccucuucaucggcauccucuucaucauccuccggaagcggcagggcagccggggcgccaugggccacuacgugcucgccgagcgggag

SEQ ID NO: 18 - RNA, 1044 nt, Artificial Sequence
  HFE Coding Sequence - Codon-Optimized
augggcccccgggcccggcccgcccugcugcugcugaugcugcugcagaccgccgugcugcagggccggcugcugcggagccacagccugcacuaccuguucaugggcgccagcgagcaggaccugggccugagccuguucgaggcccugggcuacguggacgaccagcuguucguguucuacgaccacgagagccggcggguggagccccggacccccugggugagcagccggaucagcagccagauguggcugcagcugagccagagccugaagggcugggaccacauguucaccguggacuucuggaccaucauggagaaucacaaucacagcaaggagagccacacccugcaggugauccugggcugcgagaugcaggaggacaauagcaccgagggcuacuggaaguacggcuacgacggccaggaccaccuggaguucugccccgacacccuggacuggcgggccgccgagccccgggccuggcccaccaagcuggagugggagcggcacaagauccgggcccggcagaaucgggccuaccuggagcgggacugccccgcccagcugcagcagcugcuggagcugggccggggcgugcuggaccagcaggugcccccccuggugaaggugacccaccacgugaccagcagcgugaccacccugcggugccgggcccugaauuacuacccccagaauaucaccaugaaguggcugaaggacaagcagcccauggacgccaaggaguucgagcccaaggacgugcugcccaauggcgacggcaccuaccagggcuggaucacccuggccgugccccccggcgaggagcagcgguacaccugccagguggagcaccccggccuggaccagccccugaucgugaucugggagcccagccccagcggcacccuggugaucggcgugaucagcggcaucgccguguucguggugauccuguucaucggcauccuguucaucauccugcggaagcggcagggcagccggggcgccaugggccacuacgugcuggccgagcgggag

SEQ ID NO: 19 - RNA, 1044 nt, Artificial Sequence
  HFE Coding Sequence - Codon-Optimized
augggcccucgggcccggccugcccugcugcugcugaugcugcugcagaccgccgugcugcagggccggcugcugcggagccacagccugcacuaccuguucaugggcgccagcgagcaggaccugggccugagccuguucgaggcccugggcuacguggacgaccagcuguucguguucuacgaccacgagagccggcggguggagccucggaccccuugggugagcagccggaucagcagccagauguggcugcagcugagccagagccugaagggcugggaccacauguucaccguggacuucuggaccaucauggagaaccacaaccacagcaaggagagccacacccugcaggugauccugggcugcgagaugcaggaggacaacagcaccgagggcuacuggaaguacggcuacgacggccaggaccaccuggaguucugcccugacacccuggacuggcgggccgccgagccucgggccuggccuaccaagcuggagugggagcggcacaagauccgggcccggcagaaccgggccuaccuggagcgggacugcccugcccagcugcagcagcugcuggagcugggccggggcgugcuggaccagcaggugccuccucuggugaaggugacccaccacgugaccagcagcgugaccacccugcggugccgggcccugaacuacuacccucagaacaucaccaugaaguggcugaaggacaagcagccuauggacgccaaggaguucgagccuaaggacgugcugccuaacggcgacggcaccuaccagggcuggaucacccuggccgugccuccuggcgaggagcagcgguacaccugccagguggagcacccuggccuggaccagccucugaucgugaucugggagccuagcccuagcggcacccuggugaucggcgugaucagcggcaucgccguguucguggugauccuguucaucggcauccuguucaucauccugcggaagcggcagggcagccggggcgccaugggccacuacgugcuggccgagcgggag

SEQ ID NO: 20 - RNA, 1044 nt, Artificial Sequence
  HFE Coding Sequence - Codon-Optimized
augggcccccgggcccggcccgcccugcugcugcugaugcugcugcaaaccgccgugcugcaaggccggcugcugcggagccacagccugcacuaccuguucaugggcgccagcgagcaagaccugggccugagccuguucgaggcccugggcuacguggacgaccaacuguucguguucuacgaccacgagagccggcggguggagccccggacccccugggugagcagccggaucagcagccaaauguggcugcaacugagccaaagccugaagggcugggaccacauguucaccguggacuucuggaccaucauggagaaccacaaccacagcaaggagagccacacccugcaagugauccugggcugcgagaugcaagaggacaacagcaccgagggcuacuggaaguacggcuacgacggccaagaccaccuggaguucugccccgacacccuggacuggcgggccgccgagccccgggccuggcccaccaagcuggagugggagcggcacaagauccgggcccggcaaaaccgggccuaccuggagcgggacugccccgcccaacugcaacaacugcuggagcugggccggggcgugcuggaccaacaagugcccccccuggugaaggugacccaccacgugaccagcagcgugaccacccugcggugccgggcccugaacuacuacccccaaaacaucaccaugaaguggcugaaggacaagcaacccauggacgccaaggaguucgagcccaaggacgugcugcccaacggcgacggcaccuaccaaggcuggaucacccuggccgugccccccggcgaggagcaacgguacaccugccaaguggagcaccccggccuggaccaaccccugaucgugaucugggagcccagccccagcggcacccuggugaucggcgugaucagcggcaucgccguguucguggugauccuguucaucggcauccuguucaucauccugcggaagcggcaaggcagccggggcgccaugggccacuacgugcuggccgagcgggag

SEQ ID NO: 21 - RNA, 1044 nt, Artificial Sequence
  HFE Coding Sequence - Codon-Optimized
augggccccagggccaggcccgcccugcugcugcugaugcugcugcagaccgccgugcugcagggcaggcugcugaggagccacagccugcacuaccuguucaugggcgccagcgagcaggaccugggccugagccuguucgaggcccugggcuacguggacgaccagcuguucguguucuacgaccacgagagcaggaggguggagcccaggacccccugggugagcagcaggaucagcagccagauguggcugcagcugagccagagccugaagggcugggaccacauguucaccguggacuucuggaccaucauggagaaccacaaccacagcaaggagagccacacccugcaggugauccugggcugcgagaugcaggaggacaacagcaccgagggcuacuggaaguacggcuacgacggccaggaccaccuggaguucugccccgacacccuggacuggagggccgccgagcccagggccuggcccaccaagcuggagugggagaggcacaagaucagggccaggcagaacagggccuaccuggagagggacugccccgcccagcugcagcagcugcuggagcugggcaggggcgugcuggaccagcaggugcccccccuggugaaggugacccaccacgugaccagcagcgugaccacccugaggugcagggcccugaacuacuacccccagaacaucaccaugaaguggcugaaggacaagcagcccauggacgccaaggaguucgagcccaaggacgugcugcccaacggcgacggcaccuaccagggcuggaucacccuggccgugccccccggcgaggagcagagguacaccugccagguggagcaccccggccuggaccagccccugaucgugaucugggagcccagccccagcggcacccuggugaucggcgugaucagcggcaucgccguguucguggugauccuguucaucggcauccuguucaucauccugaggaagaggcagggcagcaggggcgccaugggccacuacgugcuggccgagagggag

SEQ ID NO: 22 - RNA, 1044 nt, Artificial Sequence
  HFE Coding Sequence - Codon-Optimized
augggcccccgggcccggcccgcccugcugcugcugaugcugcugcagaccgccgugcugcagggccggcugcugcggucccacucccugcacuaccuguucaugggcgccuccgagcaggaccugggccugucccuguucgaggcccugggcuacguggacgaccagcuguucguguucuacgaccacgagucccggcggguggagccccggacccccuggguguccucccggaucuccucccagauguggcugcagcugucccagucccugaagggcugggaccacauguucaccguggacuucuggaccaucauggagaaccacaaccacuccaaggagucccacacccugcaggugauccugggcugcgagaugcaggaggacaacuccaccgagggcuacuggaaguacggcuacgacggccaggaccaccuggaguucugccccgacacccuggacuggcgggccgccgagccccgggccuggcccaccaagcuggagugggagcggcacaagauccgggcccggcagaaccgggccuaccuggagcgggacugccccgcccagcugcagcagcugcuggagcugggccggggcgugcuggaccagcaggugcccccccuggugaaggugacccaccacgugaccuccuccgugaccacccugcggugccgggcccugaacuacuacccccagaacaucaccaugaaguggcugaaggacaagcagcccauggacgccaaggaguucgagcccaaggacgugcugcccaacggcgacggcaccuaccagggcuggaucacccuggccgugccccccggcgaggagcagcgguacaccugccagguggagcaccccggccuggaccagccccugaucgugaucugggagcccucccccuccggcacccuggugaucggcgugaucuccggcaucgccguguucguggugauccuguucaucggcauccuguucaucauccugcggaagcggcagggcucccggggcgccaugggccacuacgugcuggccgagcgggag

SEQ ID NO: 23 - RNA, 1044 nt, Artificial Sequence
  HFE Coding Sequence - Codon-Optimized
augggcccccgggcccggcccgcccugcugcugcugaugcugcugcagacagccgugcugcagggccggcugcugcggagccacagccugcacuaccuguucaugggcgccagcgagcaggaccugggccugagccuguucgaggcccugggcuacguggacgaccagcuguucguguucuacgaccacgagagccggcggguggagccccggacacccugggugagcagccggaucagcagccagauguggcugcagcugagccagagccugaagggcugggaccacauguucacaguggacuucuggacaaucauggagaaccacaaccacagcaaggagagccacacacugcaggugauccugggcugcgagaugcaggaggacaacagcacagagggcuacuggaaguacggcuacgacggccaggaccaccuggaguucugccccgacacacuggacuggcgggccgccgagccccgggccuggcccacaaagcuggagugggagcggcacaagauccgggcccggcagaaccgggccuaccuggagcgggacugccccgcccagcugcagcagcugcuggagcugggccggggcgugcuggaccagcaggugcccccccuggugaaggugacacaccacgugacaagcagcgugacaacacugcggugccgggcccugaacuacuacccccagaacaucacaaugaaguggcugaaggacaagcagcccauggacgccaaggaguucgagcccaaggacgugcugcccaacggcgacggcacauaccagggcuggaucacacuggccgugccccccggcgaggagcagcgguacacaugccagguggagcaccccggccuggaccagccccugaucgugaucugggagcccagccccagcggcacacuggugaucggcgugaucagcggcaucgccguguucguggugauccuguucaucggcauccuguucaucauccugcggaagcggcagggcagccggggcgccaugggccacuacgugcuggccgagcgggag

SEQ ID NO: 24 - RNA, 1044 nt, Artificial Sequence
  HFE Coding Sequence - Codon-Optimized
augggcccccgggcccggcccgcccugcugcugcugaugcugcugcagaccgccguccugcagggccggcugcugcggagccacagccugcacuaccuguucaugggcgccagcgagcaggaccugggccugagccuguucgaggcccugggcuacgucgacgaccagcuguucgucuucuacgaccacgagagccggcgggucgagccccggacccccugggucagcagccggaucagcagccagauguggcugcagcugagccagagccugaagggcugggaccacauguucaccgucgacuucuggaccaucauggagaaccacaaccacagcaaggagagccacacccugcaggucauccugggcugcgagaugcaggaggacaacagcaccgagggcuacuggaaguacggcuacgacggccaggaccaccuggaguucugccccgacacccuggacuggcgggccgccgagccccgggccuggcccaccaagcuggagugggagcggcacaagauccgggcccggcagaaccgggccuaccuggagcgggacugccccgcccagcugcagcagcugcuggagcugggccggggcguccuggaccagcagguccccccccuggucaaggucacccaccacgucaccagcagcgucaccacccugcggugccgggcccugaacuacuacccccagaacaucaccaugaaguggcugaaggacaagcagcccauggacgccaaggaguucgagcccaaggacguccugcccaacggcgacggcaccuaccagggcuggaucacccuggccgucccccccggcgaggagcagcgguacaccugccaggucgagcaccccggccuggaccagccccugaucgucaucugggagcccagccccagcggcacccuggucaucggcgucaucagcggcaucgccgucuucgucgucauccuguucaucggcauccuguucaucauccugcggaagcggcagggcagccggggcgccaugggccacuacguccuggccgagcgggag

SEQ ID NO: 25 - RNA, 1044 nt, Artificial Sequence
  HFE Coding Sequence - Codon-Optimized
augggcccccgggcccggcccgcccugcugcugcugaugcugcugcagaccgccgugcugcagggccggcugcugcggagccacagccugcacuaucuguucaugggcgccagcgagcaggaccugggccugagccuguucgaggcccugggcuauguggacgaccagcuguucguguucuaugaccacgagagccggcggguggagccccggacccccugggugagcagccggaucagcagccagauguggcugcagcugagccagagccugaagggcugggaccacauguucaccguggacuucuggaccaucauggagaaccacaaccacagcaaggagagccacacccugcaggugauccugggcugcgagaugcaggaggacaacagcaccgagggcuauuggaaguauggcuaugacggccaggaccaccuggaguucugccccgacacccuggacuggcgggccgccgagccccgggccuggcccaccaagcuggagugggagcggcacaagauccgggcccggcagaaccgggccuaucuggagcgggacugccccgcccagcugcagcagcugcuggagcugggccggggcgugcuggaccagcaggugcccccccuggugaaggugacccaccacgugaccagcagcgugaccacccugcggugccgggcccugaacuauuauccccagaacaucaccaugaaguggcugaaggacaagcagcccauggacgccaaggaguucgagcccaaggacgugcugcccaacggcgacggcaccuaucagggcuggaucacccuggccgugccccccggcgaggagcagcgguauaccugccagguggagcaccccggccuggaccagccccugaucgugaucugggagcccagccccagcggcacccuggugaucggcgugaucagcggcaucgccguguucguggugauccuguucaucggcauccuguucaucauccugcggaagcggcagggcagccggggcgccaugggccacuaugugcuggccgagcgggag

SEQ ID NO: 26 - RNA, 1044 nt, Artificial Sequence
  HFE Coding Sequence - Codon-Optimized
auggggccuagggcuaggccugcucugcugcugcugaugcugcugcagacagcugugcugcaggggaggcugcugaggucucauucucugcauuaucuguuuaugggggcuucugagcaggaucuggggcugucucuguuugaggcucugggguauguggaugaucagcuguuuguguuuuaugaucaugagucuaggaggguggagccuaggacaccuugggugucuucuaggauuucuucucagauguggcugcagcugucucagucucugaaggggugggaucauauguuuacaguggauuuuuggacaauuauggagaaucauaaucauucuaaggagucucauacacugcaggugauucuggggugugagaugcaggaggauaauucuacagagggguauuggaaguauggguaugaugggcaggaucaucuggaguuuuguccugauacacuggauuggagggcugcugagccuagggcuuggccuacaaagcuggagugggagaggcauaagauuagggcuaggcagaauagggcuuaucuggagagggauuguccugcucagcugcagcagcugcuggagcuggggaggggggugcuggaucagcaggugccuccucuggugaaggugacacaucaugugacaucuucugugacaacacugagguguagggcucugaauuauuauccucagaauauuacaaugaaguggcugaaggauaagcagccuauggaugcuaaggaguuugagccuaaggaugugcugccuaauggggaugggacauaucagggguggauuacacuggcugugccuccuggggaggagcagagguauacaugucagguggagcauccugggcuggaucagccucugauugugauuugggagccuucuccuucugggacacuggugauuggggugauuucugggauugcuguguuuguggugauucuguuuauugggauucuguuuauuauucugaggaagaggcaggggucuaggggggcuauggggcauuaugugcuggcugagagggag

SEQ ID NO: 27 - RNA, 1044 nt, Artificial Sequence
  HFE Coding Sequence - Codon-Optimized
auggggccaagggcaaggccagcacugcugcugcugaugcugcugcagacagcagugcugcaggggaggcugcugaggucacauucacugcauuaucuguuuaugggggcaucagagcaggaucuggggcugucacuguuugaggcacugggguauguggaugaucagcuguuuguguuuuaugaucaugagucaaggaggguggagccaaggacaccaugggugucaucaaggauuucaucacagauguggcugcagcugucacagucacugaaggggugggaucauauguuuacaguggauuuuuggacaauuauggagaaucauaaucauucaaaggagucacauacacugcaggugauucuggggugugagaugcaggaggauaauucaacagagggguauuggaaguauggguaugaugggcaggaucaucuggaguuuuguccagauacacuggauuggagggcagcagagccaagggcauggccaacaaagcuggagugggagaggcauaagauuagggcaaggcagaauagggcauaucuggagagggauuguccagcacagcugcagcagcugcuggagcuggggaggggggugcuggaucagcaggugccaccacuggugaaggugacacaucaugugacaucaucagugacaacacugagguguagggcacugaauuauuauccacagaauauuacaaugaaguggcugaaggauaagcagccaauggaugcaaaggaguuugagccaaaggaugugcugccaaauggggaugggacauaucagggguggauuacacuggcagugccaccaggggaggagcagagguauacaugucagguggagcauccagggcuggaucagccacugauugugauuugggagccaucaccaucagggacacuggugauuggggugauuucagggauugcaguguuuguggugauucuguuuauugggauucuguuuauuauucugaggaagaggcaggggucaaggggggcaauggggcauuaugugcuggcagagagggag

SEQ ID NO: 28 - RNA, 1044 nt, Artificial Sequence
  HFE Coding Sequence - Codon-Optimized
augggcccaagggccaggccagcucuucuucuucugaugcuucuucagaccgcuguccugcaggggaggcuucugaggucacacucucugcacuaccuuuucaugggggccucagagcaggaccuugggcuuucccuuuuugaagcucuuggcuacguggaugaccagcuguucguguucuaugaucaugagaguaggaggguggagcccaggacuccuuggguuuccaguaggauuucaagccagauguggcugcagcugagucagagucugaaagggugggaucacauguucacuguugacuucuggacuauuauggaaaaucacaaccacagcaaggagucccacacccugcaggucauccugggcugugaaaugcaagaagacaacaguaccgagggcuacuggaaguacggguaugaugggcaggaccaccuugaauucugcccugacacccuggauuggagggcugcugaacccagggccuggcccaccaagcuggagugggaaaggcacaagauuagggccaggcagaacagggccuaccuggagagggacugcccugcucagcugcagcagcuucuggagcuggggagggggguucuugaccaacaagugccuccucuugugaaggugacccaucaugugaccucuucagugaccacucuuagguguagggcccuuaacuacuacccccagaacaucaccaugaaguggcugaaggauaagcagccuauggaugccaaggaguucgaaccuaaagacguacuucccaauggggaugggaccuaccagggcuggauuacccuugcuguacccccuggggaagagcagagguauacuugccagguggagcacccuggccuggaucagccccuuauugugaucugggagcccucaccaucuggcacccuugucauuggggucaucagugggauugcuguuuuugucgucauccuuuucauugggauucuuuucauuauucuuaggaagaggcaggggucaaggggggccauggggcacuacguccuugcugaaagggag

SEQ ID NO: 29 - RNA, 1044 nt, Artificial Sequence
  HFE Coding Sequence - Codon-Optimized
augggcccuagggccaggccugcucugcugcugcugaugcugcugcagaccgcugugcugcaggggaggcugcugaggucccacucccugcacuaccuguucaugggggccuccgagcaggaccuggggcugucccuguuugaggcucugggcuacguggaugaccagcuguucguguucuacgaucaugaguccaggaggguggagcccaggaccccuuggguguccuccaggauuuccucccagauguggcugcagcugucccagucccugaaggggugggaucacauguucaccguggacuucuggaccauuauggagaaucacaaccacuccaaggagucccacacccugcaggugauccugggcugugagaugcaggaggacaacuccaccgagggcuacuggaaguacggguacgaugggcaggaccaccuggaguucugcccugacacccuggauuggagggcugcugagcccagggccuggcccaccaagcuggagugggagaggcacaagauuagggccaggcagaacagggccuaccuggagagggacugcccugcucagcugcagcagcugcuggagcuggggaggggggugcuggaccagcaggugccuccucuggugaaggugacccaucaugugaccuccuccgugaccacccugagguguagggcccugaacuacuacccccagaacaucaccaugaaguggcugaaggauaagcagccuauggaugccaaggaguucgagccuaaggacgugcugcccaauggggaugggaccuaccagggcuggauuacccuggcugugcccccuggggaggagcagagguacaccugccagguggagcacccuggccuggaucagccccugauugugaucugggagcccuccccuuccggcacccuggugauuggggugaucuccgggauugcuguguuuguggugauccuguucauugggauucuguucauuauucugaggaagaggcagggguccaggggggccauggggcacuacgugcuggcugagagggag

SEQ ID NO: 30 - RNA, 1044 nt, Artificial Sequence
  HFE Coding Sequence - Codon-Optimized
augggcccccgggccagacccgcccugcuccuccugaugcugcuccagaccgccguccugcaggggcggcuccugcgcucccauucucugcauuaccucuuuaugggggccuccgaacaggaccuggggcugucccucuucgaggcucucggcuacguggaugaccagcuguuuguguuuuaugaucacgaaagucggcgcguggaaccccggacccccuggguguccaguaggaucuccagccagauguggcugcagcugagucagagucugaaagggugggaucauauguuuaccguggacuuuuggaccaucauggagaaucauaaccauagcaaggaaucccauacccugcaggucauucugggcugcgagaugcaggaggacaacaguaccgaaggcuacuggaaguacggguaugaugggcaggaccaucuggaguuuugcccugacacacuggauuggagggcugcugagcccagagccuggcccaccaagcuggaaugggagagacauaagauccgcgccagacagaacagagccuaccuggaaagagacugcccugcucagcugcagcagcuccuggaacuggggaggggggugcucgaccagcaggugccuccucucgugaaggugacacaccacgugaccucuuccgugaccacccuccgcugccgcgcccucaacuacuacccccagaacauuaccaugaaguggcugaaggauaagcagcccauggaugccaaggaauuugagccuaaagacguccuccccaauggggaugggaccuaccagggcuggauuacccucgcuguccccccuggggaggaacagagguauacaugccagguggaacaucccggccuggaucagccccucaucgugauuugggaacccucccccucuggcacccucgucaucggcgucauuaguggcaucgcuguguucgucgucauucucuuuaucggcauccucuuuauuauucucagaaagagacagggguccaggggcgccauggggcauuacguccucgcugagcgcgaa

SEQ ID NO: 31 - RNA, 1044 nt, Artificial Sequence
  HFE Coding Sequence - Codon-Optimized
auggggccccgggcccggcccgcucugcuccuccucaugcugcugcagaccgcuguccuccagggccggcugcuccggucccauucccuccauuaucucuuuaugggggccuccgaacaggaucuggggcugagccuguucgaggcucugggguaugucgacgaucagcucuuugucuuuuacgaccacgaaagccggcgggucgaaccccggacucccugggugagcagcaggaucuccagccagauguggcuccagcucagccagagccucaagggcugggaccauauguuuacuguggauuuuuggacuaucauggagaaccauaaucauagcaaagaaagccauacccuccaggucauucucgggugcgagaugcaagaggauaauagcaccgaaggguauuggaaauauggcuacgacggccaggaucaucuggaguuuuguccugauacccucgacuggagggccgccgagccccgggccuggcccaccaaacucgaaugggagcggcauaaaauccgggcccggcagaaucgggccuaucucgaacgggauuguccugcccagcuccagcagcugcucgaacucggcaggggggugcuggaucaacaagucccuccucuggucaaagucacccaccacgucaccuccuccgucaccacucuccggugccgggcccugaauuauuauccccagaauauuaccaugaaauggcucaaagacaaacagcccauggacgccaaagaauuugagccuaaggauguccugcccaacggcgacggcaccuaucagggguggauuacccuggcuguccccccuggcgaggaacagagguacacuugucaggucgaacaucccgggcucgaccagccccucaucgucauuugggaacccucccccuccgggacccucgucaucggcgucauuagcggcaucgcuguguucgucgucauucuguuuaucggcauccuguuuauuauucuccggaaacggcagggguccaggggcgccaugggccauuauguccucgcugagcgggaa

SEQ ID NO: 32 - protein, 348 aa, Homo sapiens
MGPRARPALLLLMLLQTAVLQGRLLRSHSLHYLFMGASEQDLGLSLFEALGYVDDQLFVFYDHESRRVEPRTPWVSSRISSQMWLQLSQSLKGWDHMFTVDFWTIMENHNHSKESHTLQVILGCEMQEDNSTEGYWKYGYDGQDHLEFCPDTLDWRAAEPRAWPTKLEWERHKIRARQNRAYLERDCPAQLQQLLELGRGVLDQQVPPLVKVTHHVTSSVTTLRCRALNYYPQNITMKWLKDKQPMDAKEFEPKDVLPNGDGTYQGWITLAVPPGEEQRYTCQVEHPGLDQPLIVIWEPSPSGTLVIGVISGIAVFVVILFIGILFIILRKRQGSRGAMGHYVLAERE

SEQ ID NO: 33 - RNA, 46 nt, Artificial Sequence
  5′ UTR
ggaaauaagagagaaaagaagaguaagaagaaauauaagagccacc

SEQ ID NO: 34 - RNA, 129 nt, Artificial Sequence
  5′ UTR - Tobacco Etch Virus
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauag

SEQ ID NO: 35 - RNA, 105 nt, Artificial Sequence
  3′ UTR
uaauuaagcugccuucugcggggcuugccuucuggccaugcccuucuucucucccuugcaccuguaccucuuggucuuugaauaaagccugaguaggaagucuag

SEQ ID NO: 36 - RNA, 158 nt, Artificial Sequence
  3′ UTR - Xenopus beta-globin
cuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacau

SEQ ID NO: 37 - DNA, 1044 bp, Artificial Sequence
  HFE Coding Sequence - Codon-Optimized DNA
atgggcccccgggcccggcccgccctgctgctgctgatgctgctgcagaccgccgtgctgcagggccggctgctgcggagccacagcctgcactacctgttcatgggcgccagcgagcaggacctgggcctgagcctgttcgaggccctgggctacgtggacgaccagctgttcgtgttctacgaccacgagagccggcgggtggagccccggaccccctgggtgagcagccggatcagcagccagatgtggctgcagctgagccagagcctgaagggctgggaccacatgttcaccgtggacttctggaccatcatggagaaccacaaccacagcaaggagagccacaccctgcaggtgatcctgggctgcgagatgcaggaggacaacagcaccgagggctactggaagtacggctacgacggccaggaccacctggagttctgccccgacaccctggactggcgggccgccgagccccgggcctggcccaccaagctggagtgggagcggcacaagatccgggcccggcagaaccgggcctacctggagcgggactgccccgcccagctgcagcagctgctggagctgggccggggcgtgctggaccagcaggtgccccccctggtgaaggtgacccaccacgtgaccagcagcgtgaccaccctgcggtgccgggccctgaactactacccccagaacatcaccatgaagtggctgaaggacaagcagcccatggacgccaaggagttcgagcccaaggacgtgctgcccaacggcgacggcacctaccagggctggatcaccctggccgtgccccccggcgaggagcagcggtacacctgccaggtggagcaccccggcctggaccagcccctgatcgtgatctgggagcccagccccagcggcaccctggtgatcggcgtgatcagcggcatcgccgtgttcgtggtgatcctgttcatcggcatcctgttcatcatcctgcggaagcggcagggcagccggggcgccatgggccactacgtgctggccgagcgggag

SEQ ID NO: 38 - DNA, 1044 bp, Artificial Sequence
  HFE Coding Sequence - Codon-Optimized DNA
atgggcccccgggccaggcccgccctgctgctgctgatgctgttgcagaccgccgtgctgcagggccggttgctgcggtcccactccctgcactacctgttcatgggcgcctccgagcaggacctgggcctgtccttgttcgaggccttgggctacgtggacgaccagctgttcgtgttctacgaccacgagagccggcgggtggagccccggaccccctgggtgtccagcaggatctccagccagatgtggctgcagctgagccagagcctgaagggctgggaccacatgttcaccgtggacttctggaccatcatggagaaccacaaccacagcaaggagtcccacaccctgcaggtgatcctgggctgcgagatgcaggaggacaacagcaccgagggctactggaagtacggctacgacggccaggaccacctggagttctgccccgacaccctggactggagggccgccgagcccagggcctggcccaccaagctggagtgggagaggcacaagatccgggccaggcagaacagggcctacctggagagggactgccccgcccagctgcagcagttgctggagctgggcaggggcgtgttggaccagcaggtgccccccttggtgaaggtgacccaccacgtgacctcctccgtgaccaccctgcggtgccgggccttgaactactacccccagaacatcaccatgaagtggctgaaggacaagcagcccatggacgccaaggagttcgagcccaaggacgtgttgcccaacggcgacggcacctaccagggctggatcaccttggccgtgccccccggcgaggagcagaggtacacctgccaggtggagcaccccggcctggaccagcccctgatcgtgatctgggagccctccccctccggcaccctggtgatcggcgtgatcagcggcatcgccgtgttcgtggtgatcttgttcatcggcatcttgttcatcatcttgaggaagaggcagggctccaggggcgccatgggccactacgtgttggccgagcgggag

SEQ ID NO: 39 - DNA, 1044 bp, Artificial Sequence
  HFE Coding Sequence - Codon-Optimized DNA
atggggccgagggcgaggccggcgctcctcctcctcatgctcctccaaacggcggtgctccaagggaggctcctcaggagccacagcctccactacctcttcatgggggcgagcgaacaagacctcgggctcagcctcttcgaagcgctcgggtacgtggacgaccaactcttcgtgttctacgaccacgaaagcaggagggtggaaccgaggacgccgtgggtgagcagcaggatcagcagccaaatgtggctccaactcagccaaagcctcaaagggtgggaccacatgttcacggtggacttctggacgatcatggaaaaccacaaccacagcaaagaaagccacacgctccaagtgatcctcgggtgcgaaatgcaagaagacaacagcacggaagggtactggaaatacgggtacgacgggcaagaccacctcgaattctgcccggacacgctcgactggagggcggcggaaccgagggcgtggccgacgaaactcgaatgggaaaggcacaaaatcagggcgaggcaaaacagggcgtacctcgaaagggactgcccggcgcaactccaacaactcctcgaactcgggaggggggtgctcgaccaacaagtgccgccgctcgtgaaagtgacgcaccacgtgacgagcagcgtgacgacgctcaggtgcagggcgctcaactactacccgcaaaacatcacgatgaaatggctcaaagacaaacaaccgatggacgcgaaagaattcgaaccgaaagacgtgctcccgaacggggacgggacgtaccaagggtggatcacgctcgcggtgccgccgggggaagaacaaaggtacacgtgccaagtggaacacccggggctcgaccaaccgctcatcgtgatctgggaaccgagcccgagcgggacgctcgtgatcggggtgatcagcgggatcgcggtgttcgtggtgatcctcttcatcgggatcctcttcatcatcctcaggaaaaggcaagggagcaggggggcgatggggcactacgtgctcgcggaaagggaa

SEQ ID NO: 40 - DNA, 1044 bp, Artificial Sequence
  HFE Coding Sequence - Codon-Optimized DNA
atggggccaagggcaaggccagcactactactactaatgctactacaaacagcagtactacaaggtaggctactaaggtcacattcactacattatctatttatgggtgcatcagaacaagatctaggtctatcactatttgaagcactagggtatgtggatgatcagctgtttgtgttttatgatcatgagtctaggagggtggagcctaggacaccttgggtgtcttctaggatttcttctcagatgtggctgcagctgtctcagtctctgaaggggtgggatcatatgtttacagtggatttttggacaattatggagaatcataatcattctaaggagtctcatacactgcaggtgattctggggtgtgagatgcaggaggataattctacagaggggtattggaagtatgggtatgatgggcaggatcatctggagttttgtcctgatacactggattggagggctgctgagcctagggcttggcctacaaagctggagtgggagaggcataagattagggctaggcagaatagggcttatctggagagggattgtcctgctcagctgcagcagctgctggagctggggaggggggtgctggatcagcaggtgcctcctctggtgaaggtgacacatcatgtgacatcttctgtgacaacactgaggtgtagggctctgaattattatcctcagaatattacaatgaagtggctgaaggataagcagcctatggatgctaaggagtttgagcctaaggatgtgctgcctaatggggatgggacatatcaggggtggattacactggctgtgcctcctggggaggagcagaggtatacatgtcaggtggagcatcctgggctggatcagcctctgattgtgatttgggagccttctccttctgggacactggtgattggggtgatttctgggattgctgtgtttgtggtgattctgtttattgggattctgtttattattctgaggaagaggcaggggtctaggggggctatggggcattatgtgctggctgagagggag

SEQ ID NO: 41 - DNA, 1044 bp, Artificial Sequence
  HFE Coding Sequence - Codon-Optimized DNA
atgggcccccgggctcggcccgctctgctgctgctgatgctgctgcagaccgctgtgctgcagggccggctgctgcggagccacagcctgcactacctgttcatgggcgctagcgagcaggacctgggcctgagcctgttcgaggctctgggctacgtggacgaccagctgttcgtgttctacgaccacgagagccggcgggtggagccccggaccccctgggtgagcagccggatcagcagccagatgtggctgcagctgagccagagcctgaagggctgggaccacatgttcaccgtggacttctggaccatcatggagaaccacaaccacagcaaggagagccacaccctgcaggtgatcctgggctgcgagatgcaggaggacaacagcaccgagggctactggaagtacggctacgacggccaggaccacctggagttctgccccgacaccctggactggcgggctgctgagccccgggcttggcccaccaagctggagtgggagcggcacaagatccgggctcggcagaaccgggcttacctggagcgggactgccccgctcagctgcagcagctgctggagctgggccggggcgtgctggaccagcaggtgccccccctggtgaaggtgacccaccacgtgaccagcagcgtgaccaccctgcggtgccgggctctgaactactacccccagaacatcaccatgaagtggctgaaggacaagcagcccatggacgctaaggagttcgagcccaaggacgtgctgcccaacggcgacggcacctaccagggctggatcaccctggctgtgccccccggcgaggagcagcggtacacctgccaggtggagcaccccggcctggaccagcccctgatcgtgatctgggagcccagccccagcggcaccctggtgatcggcgtgatcagcggcatcgctgtgttcgtggtgatcctgttcatcggcatcctgttcatcatcctgcggaagcggcagggcagccggggcgctatgggccactacgtgctggctgagcgggag

SEQ ID NO: 42 - DNA, 1044 bp, Artificial Sequence
  HFE Coding Sequence - Codon-Optimized DNA
atgggcccccgggcccggcccgccctgctgctgctgatgctgctgcagaccgccgtgctgcagggccggctgctgcggagccacagcctgcactacctgttcatgggcgccagcgagcaggacctgggcctgagcctgttcgaggccctgggctacgtggacgaccagctgttcgtgttctacgaccacgagagccggcgggtggagccccggaccccctgggtgagcagccggatcagcagccagatgtggctgcagctgagccagagcctgaagggctgggaccacatgttcaccgtggacttctggaccatcatggagaaccacaaccacagcaaggagagccacaccctgcaggtgatcctgggctgtgagatgcaggaggacaacagcaccgagggctactggaagtacggctacgacggccaggaccacctggagttctgtcccgacaccctggactggcgggccgccgagccccgggcctggcccaccaagctggagtgggagcggcacaagatccgggcccggcagaaccgggcctacctggagcgggactgtcccgcccagctgcagcagctgctggagctgggccggggcgtgctggaccagcaggtgccccccctggtgaaggtgacccaccacgtgaccagcagcgtgaccaccctgcggtgtcgggccctgaactactacccccagaacatcaccatgaagtggctgaaggacaagcagcccatggacgccaaggagttcgagcccaaggacgtgctgcccaacggcgacggcacctaccagggctggatcaccctggccgtgccccccggcgaggagcagcggtacacctgtcaggtggagcaccccggcctggaccagcccctgatcgtgatctgggagcccagccccagcggcaccctggtgatcggcgtgatcagcggcatcgccgtgttcgtggtgatcctgttcatcggcatcctgttcatcatcctgcggaagcggcagggcagccggggcgccatgggccactacgtgctggccgagcgggag

SEQ ID NO: 43 - DNA, 1044 bp, Artificial Sequence
  HFE Coding Sequence - Codon-Optimized DNA
atgggcccccgggcccggcccgccctgctgctgctgatgctgctgcagaccgccgtgctgcagggccggctgctgcggagccacagcctgcactacctgttcatgggcgccagcgagcaggatctgggcctgagcctgttcgaggccctgggctacgtggatgatcagctgttcgtgttctacgatcacgagagccggcgggtggagccccggaccccctgggtgagcagccggatcagcagccagatgtggctgcagctgagccagagcctgaagggctgggatcacatgttcaccgtggatttctggaccatcatggagaaccacaaccacagcaaggagagccacaccctgcaggtgatcctgggctgcgagatgcaggaggataacagcaccgagggctactggaagtacggctacgatggccaggatcacctggagttctgccccgataccctggattggcgggccgccgagccccgggcctggcccaccaagctggagtgggagcggcacaagatccgggcccggcagaaccgggcctacctggagcgggattgccccgcccagctgcagcagctgctggagctgggccggggcgtgctggatcagcaggtgccccccctggtgaaggtgacccaccacgtgaccagcagcgtgaccaccctgcggtgccgggccctgaactactacccccagaacatcaccatgaagtggctgaaggataagcagcccatggatgccaaggagttcgagcccaaggatgtgctgcccaacggcgatggcacctaccagggctggatcaccctggccgtgccccccggcgaggagcagcggtacacctgccaggtggagcaccccggcctggatcagcccctgatcgtgatctgggagcccagccccagcggcaccctggtgatcggcgtgatcagcggcatcgccgtgttcgtggtgatcctgttcatcggcatcctgttcatcatcctgcggaagcggcagggcagccggggcgccatgggccactacgtgctggccgagcgggag

SEQ ID NO: 44 - DNA, 1044 bp, Artificial Sequence
  HFE Coding Sequence - Codon-Optimized DNA
atgggcccccgggcccggcccgccctgctgctgctgatgctgctgcagaccgccgtgctgcagggccggctgctgcggagccacagcctgcactacctgttcatgggcgccagcgaacaggacctgggcctgagcctgttcgaagccctgggctacgtggacgaccagctgttcgtgttctacgaccacgaaagccggcgggtggaaccccggaccccctgggtgagcagccggatcagcagccagatgtggctgcagctgagccagagcctgaagggctgggaccacatgttcaccgtggacttctggaccatcatggaaaaccacaaccacagcaaggaaagccacaccctgcaggtgatcctgggctgcgaaatgcaggaagacaacagcaccgaaggctactggaagtacggctacgacggccaggaccacctggaattctgccccgacaccctggactggcgggccgccgaaccccgggcctggcccaccaagctggaatgggaacggcacaagatccgggcccggcagaaccgggcctacctggaacgggactgccccgcccagctgcagcagctgctggaactgggccggggcgtgctggaccagcaggtgccccccctggtgaaggtgacccaccacgtgaccagcagcgtgaccaccctgcggtgccgggccctgaactactacccccagaacatcaccatgaagtggctgaaggacaagcagcccatggacgccaaggaattcgaacccaaggacgtgctgcccaacggcgacggcacctaccagggctggatcaccctggccgtgccccccggcgaagaacagcggtacacctgccaggtggaacaccccggcctggaccagcccctgatcgtgatctgggaacccagccccagcggcaccctggtgatcggcgtgatcagcggcatcgccgtgttcgtggtgatcctgttcatcggcatcctgttcatcatcctgcggaagcggcagggcagccggggcgccatgggccactacgtgctggccgaacgggaa

SEQ ID NO: 45 - DNA, 1044 bp, Artificial Sequence
  HFE Coding Sequence - Codon-Optimized DNA
atgggcccccgggcccggcccgccctgctgctgctgatgctgctgcagaccgccgtgctgcagggccggctgctgcggagccacagcctgcactacctgtttatgggcgccagcgagcaggacctgggcctgagcctgtttgaggccctgggctacgtggacgaccagctgtttgtgttttacgaccacgagagccggcgggtggagccccggaccccctgggtgagcagccggatcagcagccagatgtggctgcagctgagccagagcctgaagggctgggaccacatgtttaccgtggacttttggaccatcatggagaaccacaaccacagcaaggagagccacaccctgcaggtgatcctgggctgcgagatgcaggaggacaacagcaccgagggctactggaagtacggctacgacggccaggaccacctggagttttgccccgacaccctggactggcgggccgccgagccccgggcctggcccaccaagctggagtgggagcggcacaagatccgggcccggcagaaccgggcctacctggagcgggactgccccgcccagctgcagcagctgctggagctgggccggggcgtgctggaccagcaggtgccccccctggtgaaggtgacccaccacgtgaccagcagcgtgaccaccctgcggtgccgggccctgaactactacccccagaacatcaccatgaagtggctgaaggacaagcagcccatggacgccaaggagtttgagcccaaggacgtgctgcccaacggcgacggcacctaccagggctggatcaccctggccgtgccccccggcgaggagcagcggtacacctgccaggtggagcaccccggcctggaccagcccctgatcgtgatctgggagcccagccccagcggcaccctggtgatcggcgtgatcagcggcatcgccgtgtttgtggtgatcctgtttatcggcatcctgtttatcatcctgcggaagcggcagggcagccggggcgccatgggccactacgtgctggccgagcgggag

SEQ ID NO: 46 - DNA, 1044 bp, Artificial Sequence
  HFE Coding Sequence - Codon-Optimized DNA
atggggccccgggcccggcccgccctgctgctgctgatgctgctgcagaccgccgtgctgcaggggcggctgctgcggagccacagcctgcactacctgttcatgggggccagcgagcaggacctggggctgagcctgttcgaggccctggggtacgtggacgaccagctgttcgtgttctacgaccacgagagccggcgggtggagccccggaccccctgggtgagcagccggatcagcagccagatgtggctgcagctgagccagagcctgaaggggtgggaccacatgttcaccgtggacttctggaccatcatggagaaccacaaccacagcaaggagagccacaccctgcaggtgatcctggggtgcgagatgcaggaggacaacagcaccgaggggtactggaagtacgggtacgacgggcaggaccacctggagttctgccccgacaccctggactggcgggccgccgagccccgggcctggcccaccaagctggagtgggagcggcacaagatccgggcccggcagaaccgggcctacctggagcgggactgccccgcccagctgcagcagctgctggagctggggcggggggtgctggaccagcaggtgccccccctggtgaaggtgacccaccacgtgaccagcagcgtgaccaccctgcggtgccgggccctgaactactacccccagaacatcaccatgaagtggctgaaggacaagcagcccatggacgccaaggagttcgagcccaaggacgtgctgcccaacggggacgggacctaccaggggtggatcaccctggccgtgccccccggggaggagcagcggtacacctgccaggtggagcaccccgggctggaccagcccctgatcgtgatctgggagcccagccccagcgggaccctggtgatcggggtgatcagcgggatcgccgtgttcgtggtgatcctgttcatcgggatcctgttcatcatcctgcggaagcggcaggggagccggggggccatggggcactacgtgctggccgagcgggag

SEQ ID NO: 47 - DNA, 1044 bp, Artificial Sequence
  HFE Coding Sequence - Codon-Optimized DNA
atgggcccccgggcccggcccgccctgctgctgctgatgctgctgcagaccgccgtgctgcagggccggctgctgcggagccatagcctgcattacctgttcatgggcgccagcgagcaggacctgggcctgagcctgttcgaggccctgggctacgtggacgaccagctgttcgtgttctacgaccatgagagccggcgggtggagccccggaccccctgggtgagcagccggatcagcagccagatgtggctgcagctgagccagagcctgaagggctgggaccatatgttcaccgtggacttctggaccatcatggagaaccataaccatagcaaggagagccataccctgcaggtgatcctgggctgcgagatgcaggaggacaacagcaccgagggctactggaagtacggctacgacggccaggaccatctggagttctgccccgacaccctggactggcgggccgccgagccccgggcctggcccaccaagctggagtgggagcggcataagatccgggcccggcagaaccgggcctacctggagcgggactgccccgcccagctgcagcagctgctggagctgggccggggcgtgctggaccagcaggtgccccccctggtgaaggtgacccatcatgtgaccagcagcgtgaccaccctgcggtgccgggccctgaactactacccccagaacatcaccatgaagtggctgaaggacaagcagcccatggacgccaaggagttcgagcccaaggacgtgctgcccaacggcgacggcacctaccagggctggatcaccctggccgtgccccccggcgaggagcagcggtacacctgccaggtggagcatcccggcctggaccagcccctgatcgtgatctgggagcccagccccagcggcaccctggtgatcggcgtgatcagcggcatcgccgtgttcgtggtgatcctgttcatcggcatcctgttcatcatcctgcggaagcggcagggcagccggggcgccatgggccattacgtgctggccgagcgggag

SEQ ID NO: 48 - DNA, 1044 bp, Artificial Sequence
  HFE Coding Sequence - Codon-Optimized DNA
atgggcccccgggcccggcccgccctgctgctgctgatgctgctgcagaccgccgtgctgcagggccggctgctgcggagccacagcctgcactacctgttcatgggcgccagcgagcaggacctgggcctgagcctgttcgaggccctgggctacgtggacgaccagctgttcgtgttctacgaccacgagagccggcgggtggagccccggaccccctgggtgagcagccggattagcagccagatgtggctgcagctgagccagagcctgaagggctgggaccacatgttcaccgtggacttctggaccattatggagaaccacaaccacagcaaggagagccacaccctgcaggtgattctgggctgcgagatgcaggaggacaacagcaccgagggctactggaagtacggctacgacggccaggaccacctggagttctgccccgacaccctggactggcgggccgccgagccccgggcctggcccaccaagctggagtgggagcggcacaagattcgggcccggcagaaccgggcctacctggagcgggactgccccgcccagctgcagcagctgctggagctgggccggggcgtgctggaccagcaggtgccccccctggtgaaggtgacccaccacgtgaccagcagcgtgaccaccctgcggtgccgggccctgaactactacccccagaacattaccatgaagtggctgaaggacaagcagcccatggacgccaaggagttcgagcccaaggacgtgctgcccaacggcgacggcacctaccagggctggattaccctggccgtgccccccggcgaggagcagcggtacacctgccaggtggagcaccccggcctggaccagcccctgattgtgatttgggagcccagccccagcggcaccctggtgattggcgtgattagcggcattgccgtgttcgtggtgattctgttcattggcattctgttcattattctgcggaagcggcagggcagccggggcgccatgggccactacgtgctggccgagcgggag

SEQ ID NO: 49 - DNA, 1044 bp, Artificial Sequence
  HFE Coding Sequence - Codon-Optimized DNA
atgggcccccgggcccggcccgccctgctgctgctgatgctgctgcagaccgccgtgctgcagggccggctgctgcggagccacagcctgcactacctgttcatgggcgccagcgagcaggacctgggcctgagcctgttcgaggccctgggctacgtggacgaccagctgttcgtgttctacgaccacgagagccggcgggtggagccccggaccccctgggtgagcagccggatcagcagccagatgtggctgcagctgagccagagcctgaaaggctgggaccacatgttcaccgtggacttctggaccatcatggagaaccacaaccacagcaaagagagccacaccctgcaggtgatcctgggctgcgagatgcaggaggacaacagcaccgagggctactggaaatacggctacgacggccaggaccacctggagttctgccccgacaccctggactggcgggccgccgagccccgggcctggcccaccaaactggagtgggagcggcacaaaatccgggcccggcagaaccgggcctacctggagcgggactgccccgcccagctgcagcagctgctggagctgggccggggcgtgctggaccagcaggtgccccccctggtgaaagtgacccaccacgtgaccagcagcgtgaccaccctgcggtgccgggccctgaactactacccccagaacatcaccatgaaatggctgaaagacaaacagcccatggacgccaaagagttcgagcccaaagacgtgctgcccaacggcgacggcacctaccagggctggatcaccctggccgtgccccccggcgaggagcagcggtacacctgccaggtggagcaccccggcctggaccagcccctgatcgtgatctgggagcccagccccagcggcaccctggtgatcggcgtgatcagcggcatcgccgtgttcgtggtgatcctgttcatcggcatcctgttcatcatcctgcggaaacggcagggcagccggggcgccatgggccactacgtgctggccgagcgggag

SEQ ID NO: 50 - DNA, 1044 bp, Artificial Sequence
  HFE Coding Sequence - Codon-Optimized DNA
atgggcccccgggcccggcccgccctcctcctcctcatgctcctccagaccgccgtgctccagggccggctcctccggagccacagcctccactacctcttcatgggcgccagcgagcaggacctcggcctcagcctcttcgaggccctcggctacgtggacgaccagctcttcgtgttctacgaccacgagagccggcgggtggagccccggaccccctgggtgagcagccggatcagcagccagatgtggctccagctcagccagagcctcaagggctgggaccacatgttcaccgtggacttctggaccatcatggagaaccacaaccacagcaaggagagccacaccctccaggtgatcctcggctgcgagatgcaggaggacaacagcaccgagggctactggaagtacggctacgacggccaggaccacctcgagttctgccccgacaccctcgactggcgggccgccgagccccgggcctggcccaccaagctcgagtgggagcggcacaagatccgggcccggcagaaccgggcctacctcgagcgggactgccccgcccagctccagcagctcctcgagctcggccggggcgtgctcgaccagcaggtgccccccctcgtgaaggtgacccaccacgtgaccagcagcgtgaccaccctccggtgccgggccctcaactactacccccagaacatcaccatgaagtggctcaaggacaagcagcccatggacgccaaggagttcgagcccaaggacgtgctccccaacggcgacggcacctaccagggctggatcaccctcgccgtgccccccggcgaggagcagcggtacacctgccaggtggagcaccccggcctcgaccagcccctcatcgtgatctgggagcccagccccagcggcaccctcgtgatcggcgtgatcagcggcatcgccgtgttcgtggtgatcctcttcatcggcatcctcttcatcatcctccggaagcggcagggcagccggggcgccatgggccactacgtgctcgccgagcgggag

SEQ ID NO: 51 - DNA, 1044 bp, Artificial Sequence
  HFE Coding Sequence - Codon-Optimized DNA
atgggcccccgggcccggcccgccctgctgctgctgatgctgctgcagaccgccgtgctgcagggccggctgctgcggagccacagcctgcactacctgttcatgggcgccagcgagcaggacctgggcctgagcctgttcgaggccctgggctacgtggacgaccagctgttcgtgttctacgaccacgagagccggcgggtggagccccggaccccctgggtgagcagccggatcagcagccagatgtggctgcagctgagccagagcctgaagggctgggaccacatgttcaccgtggacttctggaccatcatggagaatcacaatcacagcaaggagagccacaccctgcaggtgatcctgggctgcgagatgcaggaggacaatagcaccgagggctactggaagtacggctacgacggccaggaccacctggagttctgccccgacaccctggactggcgggccgccgagccccgggcctggcccaccaagctggagtgggagcggcacaagatccgggcccggcagaatcgggcctacctggagcgggactgccccgcccagctgcagcagctgctggagctgggccggggcgtgctggaccagcaggtgccccccctggtgaaggtgacccaccacgtgaccagcagcgtgaccaccctgcggtgccgggccctgaattactacccccagaatatcaccatgaagtggctgaaggacaagcagcccatggacgccaaggagttcgagcccaaggacgtgctgcccaatggcgacggcacctaccagggctggatcaccctggccgtgccccccggcgaggagcagcggtacacctgccaggtggagcaccccggcctggaccagcccctgatcgtgatctgggagcccagccccagcggcaccctggtgatcggcgtgatcagcggcatcgccgtgttcgtggtgatcctgttcatcggcatcctgttcatcatcctgcggaagcggcagggcagccggggcgccatgggccactacgtgctggccgagcgggag

SEQ ID NO: 52 - DNA, 1044 bp, Artificial Sequence
  HFE Coding Sequence - Codon-Optimized DNA
atgggccctcgggcccggcctgccctgctgctgctgatgctgctgcagaccgccgtgctgcagggccggctgctgcggagccacagcctgcactacctgttcatgggcgccagcgagcaggacctgggcctgagcctgttcgaggccctgggctacgtggacgaccagctgttcgtgttctacgaccacgagagccggcgggtggagcctcggaccccttgggtgagcagccggatcagcagccagatgtggctgcagctgagccagagcctgaagggctgggaccacatgttcaccgtggacttctggaccatcatggagaaccacaaccacagcaaggagagccacaccctgcaggtgatcctgggctgcgagatgcaggaggacaacagcaccgagggctactggaagtacggctacgacggccaggaccacctggagttctgccctgacaccctggactggcgggccgccgagcctcgggcctggcctaccaagctggagtgggagcggcacaagatccgggcccggcagaaccgggcctacctggagcgggactgccctgcccagctgcagcagctgctggagctgggccggggcgtgctggaccagcaggtgcctcctctggtgaaggtgacccaccacgtgaccagcagcgtgaccaccctgcggtgccgggccctgaactactaccctcagaacatcaccatgaagtggctgaaggacaagcagcctatggacgccaaggagttcgagcctaaggacgtgctgcctaacggcgacggcacctaccagggctggatcaccctggccgtgcctcctggcgaggagcagcggtacacctgccaggtggagcaccctggcctggaccagcctctgatcgtgatctgggagcctagccctagcggcaccctggtgatcggcgtgatcagcggcatcgccgtgttcgtggtgatcctgttcatcggcatcctgttcatcatcctgcggaagcggcagggcagccggggcgccatgggccactacgtgctggccgagcgggag

SEQ ID NO: 53 - DNA, 1044 bp, Artificial Sequence
  HFE Coding Sequence - Codon-Optimized DNA
atgggcccccgggcccggcccgccctgctgctgctgatgctgctgcaaaccgccgtgctgcaaggccggctgctgcggagccacagcctgcactacctgttcatgggcgccagcgagcaagacctgggcctgagcctgttcgaggccctgggctacgtggacgaccaactgttcgtgttctacgaccacgagagccggcgggtggagccccggaccccctgggtgagcagccggatcagcagccaaatgtggctgcaactgagccaaagcctgaagggctgggaccacatgttcaccgtggacttctggaccatcatggagaaccacaaccacagcaaggagagccacaccctgcaagtgatcctgggctgcgagatgcaagaggacaacagcaccgagggctactggaagtacggctacgacggccaagaccacctggagttctgccccgacaccctggactggcgggccgccgagccccgggcctggcccaccaagctggagtgggagcggcacaagatccgggcccggcaaaaccgggcctacctggagcgggactgccccgcccaactgcaacaactgctggagctgggccggggcgtgctggaccaacaagtgccccccctggtgaaggtgacccaccacgtgaccagcagcgtgaccaccctgcggtgccgggccctgaactactacccccaaaacatcaccatgaagtggctgaaggacaagcaacccatggacgccaaggagttcgagcccaaggacgtgctgcccaacggcgacggcacctaccaaggctggatcaccctggccgtgccccccggcgaggagcaacggtacacctgccaagtggagcaccccggcctggaccaacccctgatcgtgatctgggagcccagccccagcggcaccctggtgatcggcgtgatcagcggcatcgccgtgttcgtggtgatcctgttcatcggcatcctgttcatcatcctgcggaagcggcaaggcagccggggcgccatgggccactacgtgctggccgagcgggag

SEQ ID NO: 54 - DNA, 1044 bp, Artificial Sequence
  HFE Coding Sequence - Codon-Optimized DNA
atgggccccagggccaggcccgccctgctgctgctgatgctgctgcagaccgccgtgctgcagggcaggctgctgaggagccacagcctgcactacctgttcatgggcgccagcgagcaggacctgggcctgagcctgttcgaggccctgggctacgtggacgaccagctgttcgtgttctacgaccacgagagcaggagggtggagcccaggaccccctgggtgagcagcaggatcagcagccagatgtggctgcagctgagccagagcctgaagggctgggaccacatgttcaccgtggacttctggaccatcatggagaaccacaaccacagcaaggagagccacaccctgcaggtgatcctgggctgcgagatgcaggaggacaacagcaccgagggctactggaagtacggctacgacggccaggaccacctggagttctgccccgacaccctggactggagggccgccgagcccagggcctggcccaccaagctggagtgggagaggcacaagatcagggccaggcagaacagggcctacctggagagggactgccccgcccagctgcagcagctgctggagctgggcaggggcgtgctggaccagcaggtgccccccctggtgaaggtgacccaccacgtgaccagcagcgtgaccaccctgaggtgcagggccctgaactactacccccagaacatcaccatgaagtggctgaaggacaagcagcccatggacgccaaggagttcgagcccaaggacgtgctgcccaacggcgacggcacctaccagggctggatcaccctggccgtgccccccggcgaggagcagaggtacacctgccaggtggagcaccccggcctggaccagcccctgatcgtgatctgggagcccagccccagcggcaccctggtgatcggcgtgatcagcggcatcgccgtgttcgtggtgatcctgttcatcggcatcctgttcatcatcctgaggaagaggcagggcagcaggggcgccatgggccactacgtgctggccgagagggag

SEQ ID NO: 55 - DNA, 1044 bp, Artificial Sequence
  HFE Coding Sequence - Codon-Optimized DNA
atgggcccccgggcccggcccgccctgctgctgctgatgctgctgcagaccgccgtgctgcagggccggctgctgcggtcccactccctgcactacctgttcatgggcgcctccgagcaggacctgggcctgtccctgttcgaggccctgggctacgtggacgaccagctgttcgtgttctacgaccacgagtcccggcgggtggagccccggaccccctgggtgtcctcccggatctcctcccagatgtggctgcagctgtcccagtccctgaagggctgggaccacatgttcaccgtggacttctggaccatcatggagaaccacaaccactccaaggagtcccacaccctgcaggtgatcctgggctgcgagatgcaggaggacaactccaccgagggctactggaagtacggctacgacggccaggaccacctggagttctgccccgacaccctggactggcgggccgccgagccccgggcctggcccaccaagctggagtgggagcggcacaagatccgggcccggcagaaccgggcctacctggagcgggactgccccgcccagctgcagcagctgctggagctgggccggggcgtgctggaccagcaggtgccccccctggtgaaggtgacccaccacgtgacctcctccgtgaccaccctgcggtgccgggccctgaactactacccccagaacatcaccatgaagtggctgaaggacaagcagcccatggacgccaaggagttcgagcccaaggacgtgctgcccaacggcgacggcacctaccagggctggatcaccctggccgtgccccccggcgaggagcagcggtacacctgccaggtggagcaccccggcctggaccagcccctgatcgtgatctgggagccctccccctccggcaccctggtgatcggcgtgatctccggcatcgccgtgttcgtggtgatcctgttcatcggcatcctgttcatcatcctgcggaagcggcagggctcccggggcgccatgggccactacgtgctggccgagcgggag

SEQ ID NO: 56 - DNA, 1044 bp, Artificial Sequence
  HFE Coding Sequence - Codon-Optimized DNA
atgggcccccgggcccggcccgccctgctgctgctgatgctgctgcagacagccgtgctgcagggccggctgctgcggagccacagcctgcactacctgttcatgggcgccagcgagcaggacctgggcctgagcctgttcgaggccctgggctacgtggacgaccagctgttcgtgttctacgaccacgagagccggcgggtggagccccggacaccctgggtgagcagccggatcagcagccagatgtggctgcagctgagccagagcctgaagggctgggaccacatgttcacagtggacttctggacaatcatggagaaccacaaccacagcaaggagagccacacactgcaggtgatcctgggctgcgagatgcaggaggacaacagcacagagggctactggaagtacggctacgacggccaggaccacctggagttctgccccgacacactggactggcgggccgccgagccccgggcctggcccacaaagctggagtgggagcggcacaagatccgggcccggcagaaccgggcctacctggagcgggactgccccgcccagctgcagcagctgctggagctgggccggggcgtgctggaccagcaggtgccccccctggtgaaggtgacacaccacgtgacaagcagcgtgacaacactgcggtgccgggccctgaactactacccccagaacatcacaatgaagtggctgaaggacaagcagcccatggacgccaaggagttcgagcccaaggacgtgctgcccaacggcgacggcacataccagggctggatcacactggccgtgccccccggcgaggagcagcggtacacatgccaggtggagcaccccggcctggaccagcccctgatcgtgatctgggagcccagccccagcggcacactggtgatcggcgtgatcagcggcatcgccgtgttcgtggtgatcctgttcatcggcatcctgttcatcatcctgcggaagcggcagggcagccggggcgccatgggccactacgtgctggccgagcgggag

SEQ ID NO: 57 - DNA, 1044 bp, Artificial Sequence
  HFE Coding Sequence - Codon-Optimized DNA
atgggcccccgggcccggcccgccctgctgctgctgatgctgctgcagaccgccgtcctgcagggccggctgctgcggagccacagcctgcactacctgttcatgggcgccagcgagcaggacctgggcctgagcctgttcgaggccctgggctacgtcgacgaccagctgttcgtcttctacgaccacgagagccggcgggtcgagccccggaccccctgggtcagcagccggatcagcagccagatgtggctgcagctgagccagagcctgaagggctgggaccacatgttcaccgtcgacttctggaccatcatggagaaccacaaccacagcaaggagagccacaccctgcaggtcatcctgggctgcgagatgcaggaggacaacagcaccgagggctactggaagtacggctacgacggccaggaccacctggagttctgccccgacaccctggactggcgggccgccgagccccgggcctggcccaccaagctggagtgggagcggcacaagatccgggcccggcagaaccgggcctacctggagcgggactgccccgcccagctgcagcagctgctggagctgggccggggcgtcctggaccagcaggtcccccccctggtcaaggtcacccaccacgtcaccagcagcgtcaccaccctgcggtgccgggccctgaactactacccccagaacatcaccatgaagtggctgaaggacaagcagcccatggacgccaaggagttcgagcccaaggacgtcctgcccaacggcgacggcacctaccagggctggatcaccctggccgtcccccccggcgaggagcagcggtacacctgccaggtcgagcaccccggcctggaccagcccctgatcgtcatctgggagcccagccccagcggcaccctggtcatcggcgtcatcagcggcatcgccgtcttcgtcgtcatcctgttcatcggcatcctgttcatcatcctgcggaagcggcagggcagccggggcgccatgggccactacgtcctggccgagcgggag

SEQ ID NO: 58 - DNA, 1044 bp, Artificial Sequence
  HFE Coding Sequence - Codon-Optimized DNA
atgggcccccgggcccggcccgccctgctgctgctgatgctgctgcagaccgccgtgctgcagggccggctgctgcggagccacagcctgcactatctgttcatgggcgccagcgagcaggacctgggcctgagcctgttcgaggccctgggctatgtggacgaccagctgttcgtgttctatgaccacgagagccggcgggtggagccccggaccccctgggtgagcagccggatcagcagccagatgtggctgcagctgagccagagcctgaagggctgggaccacatgttcaccgtggacttctggaccatcatggagaaccacaaccacagcaaggagagccacaccctgcaggtgatcctgggctgcgagatgcaggaggacaacagcaccgagggctattggaagtatggctatgacggccaggaccacctggagttctgccccgacaccctggactggcgggccgccgagccccgggcctggcccaccaagctggagtgggagcggcacaagatccgggcccggcagaaccgggcctatctggagcgggactgccccgcccagctgcagcagctgctggagctgggccggggcgtgctggaccagcaggtgccccccctggtgaaggtgacccaccacgtgaccagcagcgtgaccaccctgcggtgccgggccctgaactattatccccagaacatcaccatgaagtggctgaaggacaagcagcccatggacgccaaggagttcgagcccaaggacgtgctgcccaacggcgacggcacctatcagggctggatcaccctggccgtgccccccggcgaggagcagcggtatacctgccaggtggagcaccccggcctggaccagcccctgatcgtgatctgggagcccagccccagcggcaccctggtgatcggcgtgatcagcggcatcgccgtgttcgtggtgatcctgttcatcggcatcctgttcatcatcctgcggaagcggcagggcagccggggcgccatgggccactatgtgctggccgagcgggag

SEQ ID NO: 59 - DNA, 1044 bp, Artificial Sequence
  HFE Coding Sequence - Codon-Optimized DNA
atggggcctagggctaggcctgctctgctgctgctgatgctgctgcagacagctgtgctgcaggggaggctgctgaggtctcattctctgcattatctgtttatgggggcttctgagcaggatctggggctgtctctgtttgaggctctggggtatgtggatgatcagctgtttgtgttttatgatcatgagtctaggagggtggagcctaggacaccttgggtgtcttctaggatttcttctcagatgtggctgcagctgtctcagtctctgaaggggtgggatcatatgtttacagtggatttttggacaattatggagaatcataatcattctaaggagtctcatacactgcaggtgattctggggtgtgagatgcaggaggataattctacagaggggtattggaagtatgggtatgatgggcaggatcatctggagttttgtcctgatacactggattggagggctgctgagcctagggcttggcctacaaagctggagtgggagaggcataagattagggctaggcagaatagggcttatctggagagggattgtcctgctcagctgcagcagctgctggagctggggaggggggtgctggatcagcaggtgcctcctctggtgaaggtgacacatcatgtgacatcttctgtgacaacactgaggtgtagggctctgaattattatcctcagaatattacaatgaagtggctgaaggataagcagcctatggatgctaaggagtttgagcctaaggatgtgctgcctaatggggatgggacatatcaggggtggattacactggctgtgcctcctggggaggagcagaggtatacatgtcaggtggagcatcctgggctggatcagcctctgattgtgatttgggagccttctccttctgggacactggtgattggggtgatttctgggattgctgtgtttgtggtgattctgtttattgggattctgtttattattctgaggaagaggcaggggtctaggggggctatggggcattatgtgctggctgagagggag

SEQ ID NO: 60 - DNA, 1044 bp, Artificial Sequence
  HFE Coding Sequence - Codon-Optimized DNA
atggggccaagggcaaggccagcactgctgctgctgatgctgctgcagacagcagtgctgcaggggaggctgctgaggtcacattcactgcattatctgtttatgggggcatcagagcaggatctggggctgtcactgtttgaggcactggggtatgtggatgatcagctgtttgtgttttatgatcatgagtcaaggagggtggagccaaggacaccatgggtgtcatcaaggatttcatcacagatgtggctgcagctgtcacagtcactgaaggggtgggatcatatgtttacagtggatttttggacaattatggagaatcataatcattcaaaggagtcacatacactgcaggtgattctggggtgtgagatgcaggaggataattcaacagaggggtattggaagtatgggtatgatgggcaggatcatctggagttttgtccagatacactggattggagggcagcagagccaagggcatggccaacaaagctggagtgggagaggcataagattagggcaaggcagaatagggcatatctggagagggattgtccagcacagctgcagcagctgctggagctggggaggggggtgctggatcagcaggtgccaccactggtgaaggtgacacatcatgtgacatcatcagtgacaacactgaggtgtagggcactgaattattatccacagaatattacaatgaagtggctgaaggataagcagccaatggatgcaaaggagtttgagccaaaggatgtgctgccaaatggggatgggacatatcaggggtggattacactggcagtgccaccaggggaggagcagaggtatacatgtcaggtggagcatccagggctggatcagccactgattgtgatttgggagccatcaccatcagggacactggtgattggggtgatttcagggattgcagtgtttgtggtgattctgtttattgggattctgtttattattctgaggaagaggcaggggtcaaggggggcaatggggcattatgtgctggcagagagggag

SEQ ID NO: 61 - DNA, 1044 bp, Artificial Sequence
  HFE Coding Sequence - Codon-Optimized DNA
atgggcccaagggccaggccagctcttcttcttctgatgcttcttcagaccgctgtcctgcaggggaggcttctgaggtcacactctctgcactaccttttcatgggggcctcagagcaggaccttgggctttccctttttgaagctcttggctacgtggatgaccagctgttcgtgttctatgatcatgagagtaggagggtggagcccaggactccttgggtttccagtaggatttcaagccagatgtggctgcagctgagtcagagtctgaaagggtgggatcacatgttcactgttgacttctggactattatggaaaatcacaaccacagcaaggagtcccacaccctgcaggtcatcctgggctgtgaaatgcaagaagacaacagtaccgagggctactggaagtacgggtatgatgggcaggaccaccttgaattctgccctgacaccctggattggagggctgctgaacccagggcctggcccaccaagctggagtgggaaaggcacaagattagggccaggcagaacagggcctacctggagagggactgccctgctcagctgcagcagcttctggagctggggaggggggttcttgaccaacaagtgcctcctcttgtgaaggtgacccatcatgtgacctcttcagtgaccactcttaggtgtagggcccttaactactacccccagaacatcaccatgaagtggctgaaggataagcagcctatggatgccaaggagttcgaacctaaagacgtacttcccaatggggatgggacctaccagggctggattacccttgctgtaccccctggggaagagcagaggtatacttgccaggtggagcaccctggcctggatcagccccttattgtgatctgggagccctcaccatctggcacccttgtcattggggtcatcagtgggattgctgtttttgtcgtcatccttttcattgggattcttttcattattcttaggaagaggcaggggtcaaggggggccatggggcactacgtccttgctgaaagggag

SEQ ID NO: 62 - DNA, 1044 bp, Artificial Sequence
  HFE Coding Sequence - Codon-Optimized DNA
atgggccctagggccaggcctgctctgctgctgctgatgctgctgcagaccgctgtgctgcaggggaggctgctgaggtcccactccctgcactacctgttcatgggggcctccgagcaggacctggggctgtccctgtttgaggctctgggctacgtggatgaccagctgttcgtgttctacgatcatgagtccaggagggtggagcccaggaccccttgggtgtcctccaggatttcctcccagatgtggctgcagctgtcccagtccctgaaggggtgggatcacatgttcaccgtggacttctggaccattatggagaatcacaaccactccaaggagtcccacaccctgcaggtgatcctgggctgtgagatgcaggaggacaactccaccgagggctactggaagtacgggtacgatgggcaggaccacctggagttctgccctgacaccctggattggagggctgctgagcccagggcctggcccaccaagctggagtgggagaggcacaagattagggccaggcagaacagggcctacctggagagggactgccctgctcagctgcagcagctgctggagctggggaggggggtgctggaccagcaggtgcctcctctggtgaaggtgacccatcatgtgacctcctccgtgaccaccctgaggtgtagggccctgaactactacccccagaacatcaccatgaagtggctgaaggataagcagcctatggatgccaaggagttcgagcctaaggacgtgctgcccaatggggatgggacctaccagggctggattaccctggctgtgccccctggggaggagcagaggtacacctgccaggtggagcaccctggcctggatcagcccctgattgtgatctgggagccctccccttccggcaccctggtgattggggtgatctccgggattgctgtgtttgtggtgatcctgttcattgggattctgttcattattctgaggaagaggcaggggtccaggggggccatggggcactacgtgctggctgagagggag

SEQ ID NO: 63 - DNA, 1044 bp, Artificial Sequence
  HFE Coding Sequence - Codon-Optimized DNA
atgggcccccgggccagacccgccctgctcctcctgatgctgctccagaccgccgtcctgcaggggcggctcctgcgctcccattctctgcattacctctttatgggggcctccgaacaggacctggggctgtccctcttcgaggctctcggctacgtggatgaccagctgtttgtgttttatgatcacgaaagtcggcgcgtggaaccccggaccccctgggtgtccagtaggatctccagccagatgtggctgcagctgagtcagagtctgaaagggtgggatcatatgtttaccgtggacttttggaccatcatggagaatcataaccatagcaaggaatcccataccctgcaggtcattctgggctgcgagatgcaggaggacaacagtaccgaaggctactggaagtacgggtatgatgggcaggaccatctggagttttgccctgacacactggattggagggctgctgagcccagagcctggcccaccaagctggaatgggagagacataagatccgcgccagacagaacagagcctacctggaaagagactgccctgctcagctgcagcagctcctggaactggggaggggggtgctcgaccagcaggtgcctcctctcgtgaaggtgacacaccacgtgacctcttccgtgaccaccctccgctgccgcgccctcaactactacccccagaacattaccatgaagtggctgaaggataagcagcccatggatgccaaggaatttgagcctaaagacgtcctccccaatggggatgggacctaccagggctggattaccctcgctgtcccccctggggaggaacagaggtatacatgccaggtggaacatcccggcctggatcagcccctcatcgtgatttgggaaccctccccctctggcaccctcgtcatcggcgtcattagtggcatcgctgtgttcgtcgtcattctctttatcggcatcctctttattattctcagaaagagacaggggtccaggggcgccatggggcattacgtcctcgctgagcgcgaa

SEQ ID NO: 64 - DNA, 1044 bp, Artificial Sequence
  HFE Coding Sequence - Codon-Optimized DNA
atggggccccgggcccggcccgctctgctcctcctcatgctgctgcagaccgctgtcctccagggccggctgctccggtcccattccctccattatctctttatgggggcctccgaacaggatctggggctgagcctgttcgaggctctggggtatgtcgacgatcagctctttgtcttttacgaccacgaaagccggcgggtcgaaccccggactccctgggtgagcagcaggatctccagccagatgtggctccagctcagccagagcctcaagggctgggaccatatgtttactgtggatttttggactatcatggagaaccataatcatagcaaagaaagccataccctccaggtcattctcgggtgcgagatgcaagaggataatagcaccgaagggtattggaaatatggctacgacggccaggatcatctggagttttgtcctgataccctcgactggagggccgccgagccccgggcctggcccaccaaactcgaatgggagcggcataaaatccgggcccggcagaatcgggcctatctcgaacgggattgtcctgcccagctccagcagctgctcgaactcggcaggggggtgctggatcaacaagtccctcctctggtcaaagtcacccaccacgtcacctcctccgtcaccactctccggtgccgggccctgaattattatccccagaatattaccatgaaatggctcaaagacaaacagcccatggacgccaaagaatttgagcctaaggatgtcctgcccaacggcgacggcacctatcaggggtggattaccctggctgtcccccctggcgaggaacagaggtacacttgtcaggtcgaacatcccgggctcgaccagcccctcatcgtcatttgggaaccctccccctccgggaccctcgtcatcggcgtcattagcggcatcgctgtgttcgtcgtcattctgtttatcggcatcctgtttattattctccggaaacggcaggggtccaggggcgccatgggccattatgtcctcgctgagcgggaa

SEQ ID NO: 65 - RNA, 9 nt, Artificial Sequence
  Triple stop codon
auaagugaa

SEQ ID NO: 66 - RNA, 6 nt, Artificial Sequence
  Kozak sequence
gccacc

SEQ ID NO: 67 - RNA, 1208 nt, Artificial Sequence
  Encoding sequence for HFE protein
aggaaauaagagagaaaagaagaguaagaagaaauauaagagccaccaugggcccccgggcccggcccgcccugcugcugcugaugcugcugcagaccgccgugcugcagggccggcugcugcggagccacagccugcacuaccuguucaugggcgccagcgagcaggaccugggccugagccuguucgaggcccugggcuacguggacgaccagcuguucguguucuacgaccacgagagccggcggguggagccccggacccccugggugagcagccggaucagcagccagauguggcugcagcugagccagagccugaagggcugggaccacauguucaccguggacuucuggaccaucauggagaaccacaaccacagcaaggagagccacacccugcaggugauccugggcugcgagaugcaggaggacaacagcaccgagggcuacuggaaguacggcuacgacggccaggaccaccuggaguucugccccgacacccuggacuggcgggccgccgagccccgggccuggcccaccaagcuggagugggagcggcacaagauccgggcccggcagaaccgggccuaccuggagcgggacugccccgcccagcugcagcagcugcuggagcugggccggggcgugcuggaccagcaggugcccccccuggugaaggugacccaccacgugaccagcagcgugaccacccugcggugccgggcccugaacuacuacccccagaacaucaccaugaaguggcugaaggacaagcagcccauggacgccaaggaguucgagcccaaggacgugcugcccaacggcgacggcaccuaccagggcuggaucacccuggccgugccccccggcgaggagcagcgguacaccugccagguggagcaccccggccuggaccagccccugaucgugaucugggagcccagccccagcggcacccuggugaucggcgugaucagcggcaucgccguguucguggugauccuguucaucggcauccuguucaucauccugcggaagcggcagggcagccggggcgccaugggccacuacgugcuggccgagcgggagugagcggccgcuuaauuaagcugccuucugcggggcuugccuucuggccaugcccuucuucucucccuugcaccuguaccucuuggucuuugaauaaagccugaguaggaagucuag